(19) United States
(12) Patent Application Publication
(10) Pub. No.: US 2025/0041299 A1
(43) Pub. Date: Feb. 6, 2025

(54) THERAPEUTIC TARGETING OF GASTROINTESTINAL STROMAL TUMOR (GIST) BY DISRUPTING THE MENIN-MLL EPIGENETIC COMPLEX

(71) Applicant: DANA-FARBER CANCER INSTITUTE, INC., Boston, MA (US)

(72) Inventors: Scott A. ARMSTRONG, Wayland, MA (US); Matthew L. HEMMING, Boston, MA (US)

(73) Assignee: DANA-FARBER CANCER INSTITUTE, INC., Boston, MA (US)

(21) Appl. No.: 18/717,194

(22) PCT Filed: Dec. 14, 2022
(86) PCT No.: PCT/US2022/081587

§ 371 Date: Jun. 6, 2024

Related U.S. Application Data
(60) Provisional application No. 63/289,943, filed on Dec. 15, 2021.

(51) Int. Cl. A61K 31/506; A61K 31/4402; A61K 31/4545; A61K 31/713; A61P 35/00

(52) U.S. Cl. CPC A61K 31/506; A61K 31/4402; A61K 31/4545; A61K 31/713; A61P 35/00

(57) ABSTRACT

Disclosed are methods of treatment and inhibitors for gastrointestinal stromal tumor (GIST) in a subject, with an active agent that inhibits a Menin or a member of the Menin-MILL complex.

### RELATED APPLICATIONS

This application claims the benefit of priority under 35 U.S.C. § 119 (e) to U.S. Provisional Application No. 63/289,943, filed Dec. 15, 2021, which is incorporated herein by reference in its entirety.

### GOVERNMENT LICENSE RIGHTS

This invention was made with government support under grant number K08 CA245235 and UL 1TR002541 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Dec. 14, 2022, is named 52095-752001WO_SL.xml and is 51 KB bytes in size.

### BACKGROUND OF THE DISCLOSURE

Gastrointestinal stromal tumor (GIST) is a soft tissue sarcoma that can be located in any part of the digestive system, most commonly in the stomach and small intestine. GIST is characterized by recurrent activating mutations in or around the tyrosine kinases KIT proto-oncogene, receptor tyrosine kinase (KIT) or Platelet Derived Growth Factor Receptor Alpha (PDGFRA) (Corless et al., Annu. Rev. Pathol. Mech. Dis. 3:557-86 (2008), Hemming et al., Annals of Oncology. 3:557-9 (2018)).

Mutations in or around KIT and/or PDGFRA account for over 85% of GIST cases. The majority of KIT primary mutations responds to treatment with the tyrosine kinase inhibitor (TKI) imatinib. However, secondary kinase mutations arise over time, creating imatinib-resistant GIST. Sunitinib, regorafenib, and ripretinib are approved for the treatment of imatinib-resistant GIST in later lines of treatment, although resistance to these drugs also develops over time (Demetri et al., N. Engl. J. Med. 347 (7): 472-80 (2002), Blay et al., Lancet Oncol. 21 (7): 923-34 (2020)), Voss and Hager, Nat. Rev. Genet. 15 (2): 69-81 (2014), Chen and Dent, Nat. Rev. Genet. 15 (2): 93-106 (2014)).

Therefore, a treatment against multi drug-resistant GIST is urgently needed.

### SUMMARY OF THE DISCLOSURE

A first aspect of the present disclosure is directed to a method of treating gastrointestinal stromal tumor (GIST). The method entails administering to a subject a therapeutically effective amount of a Menin inhibitor. In some embodiments, the method also entails administering to the subject a therapeutically effective amount of a tyrosine kinase inhibitor (TKI) and/or a therapeutically effective amount of a MOZ inhibitor.

Another aspect of the present disclosure is a method of reducing KIT activity in vitro or in vivo. The method entails contacting a cell having an activating mutation in or around the KIT gene with a Menin inhibitor. In some embodiments, the method entails administering to the subject a therapeutically effective amount of a TKI and/or a therapeutically effective amount of a MOZ inhibitor.

Yet another aspect of the present disclosure is directed to a kit containing a therapeutically effective amount of a Menin inhibitor, a pharmaceutically acceptable carrier disposed in a suitable container, and printed instructions for using the Menin inhibitor in the treatment of GIST in a subject. In some embodiments, the kit also contains a therapeutically effective amount of a TKI and printed instructions for using the TKI in the treatment of GIST in a subject, wherein the Menin inhibitor and the TKI are contained in the same dosage form or different dosage forms that are disposed in the same or different containers. In some embodiments, the kit also contains a therapeutically effective amount of a MOZ inhibitor and printed instructions for using the MOZ inhibitor in the treatment of GIST in a subject, wherein the Menin inhibitor and the MOZ inhibitor are contained in the same dosage form or different dosage forms that are disposed in the same or different containers.

As shown in the working examples, the present inventors have shown that the Menin-MLL and MOZ chromatin regulatory complexes were enriched at GIST-relevant genes, regulated their transcription, and the transcription of the GIST epigenome. Inhibition of the Menin-MLL complex, alone or in combination with MOZ complex inhibition, decreased GIST cell proliferation by disrupting interactions with transcriptional and chromatin regulators, such as DOT1L. Menin and MOZ inhibition caused significant reductions in tumor burden in vivo. with even greater effects observed with combined Menin and KIT inhibition.

### BRIEF DESCRIPTION OF THE DRAWINGS

FIGS. 1A-IG are a series of scatter, bar, and dot plots that illustrate the identification of GIST epigenetic dependencies through genome-scale CRISPR dependency screens. FIGS. 1A and 1B are scatter plots showing correlation β-scores. FIG. 1A shows the correlation between H1 and H2 sgRNA libraries. each targeting 18.436 genes with 5 sgRNAs per library. FIG. 1B shows the correlation between GIST430 and GIST-T1 cell lines. FIG. 1C is a scatter plot showing the rank in screen and β-score merging H1 and H2 libraries and GIST cell lines. FIGS. 1D and 1E are bar plots showing relative reads for individual sgRNAs comparing baseline plasmid library sequencing to screen end result. FIG. 1D is a bar graph that shows KIT sgRNAs and FIG. 1E is a bar graph that shows MTOR sgRNAs. FIG. 1F is a dot plot comparing β-scores of pan-essential and non-essential genes. FIG. 1G is a bar plot showing 8 of the top 18 significantly enriched gene ontology terms among genes uniquely essential in GIST.

FIGS. 2A-2F are a series of scatter. Cirocs, line, and bar plots that illustrate the unique co-dependency of MOZ and the Menin-MLL complexes. FIG. 2A is a scatter plot of merged β-score in GIST-T1 and GIST430 and average CERES score of all cell lines in DepMap for chromatin modifying enzymes. FIG. 2B is a Circos plot showing overlap of the top 50 DepMap correlated dependencies of the seven chromatin modifying enzymes with enriched dependencies in GIST. FIGS. 2C and 2D are scatter plots that show Ranked Sensitivity Scores from Project Drive cell lines for Menin-MLL complex members KMT2A and ASH2L, with GIST-T1 highlighted in red. FIG. 2E is a line plot that shows growth over time assay following transduction of the indicated sgRNAs targeting Menin-MLL complex members in GIST-T1. FIG. 2F is a bar plot that shows day 21 cell count in a growth over time assay comparing GIST-T1 to GIST48B.

FIGS. 3A-31 are a series of heat maps. Venn diagrams. scatter plots, and tracks that show the genomic localization of MOZ and Menin-MLL complexes in GIST. FIG. 3A is a series of heat maps demonstrating genomic localization in GIST-T1 of H3K27ac. H3K9ac. H3K4me3. BRPF1, and KAT6A by ChIP-seq, and Menin and MLLIn by CUT&Tag. FIGS. 3B-3D are diagrams showing the overlap of MACS-defined peaks. FIG. 3B is a diagram depicting BRPF1 and KAT6A. FIG. 3C is a diagram depicting Menin and BRPF1. FIG. 3D is a Venn diagram depicting Menin and MLL1n. FIG. 3E is a scatter plot that shows the enriched genomic regions of BRPF1 binding, with TFs indicated in red. FIG. 3F is a scatter plot that shows the enriched genomic regions of Menin binding, with TFs indicated in red. FIGS. 3G-31 are tracks showing regions of genomic occupancy of the TF HAND1. MOZ complex members BRPF1 and KAT6A. Menin-MLL complex members Menin and MLL In, and histone markers H3K4me3. H3K9ac, and H3K27ac at various gene loci: with FIG. 3G showing the FOXF1 loci. FIG. 3H showing the DUSP6 loci, and FIG. 3I showing the USP1 loci.

FIGS. 4A-4F are a series of line and bar plots that demonstrate that the inhibition of Menin-MLL complex with and without MOZ complex inhibition leading to cell cycle arrest. FIG. 4A is a line plot showing a growth over time assay in GIST-T1 with the indicated concentrations of Menin inhibitor VTP-50469. FIG. 4B is a line plot showing a growth over time assay in GIST-T1 treated with VTP-50469 with or without WM-1119. each inhibitor used at 0.1 μM. FIG. 4C is a bar plot showing a day 21 cell count normalized to DMSO following treatment of GIST48B. GIST-T1 or KIT enhancer independent cell line GIST-T1/KIT^{Δe11} (endogenous KIT knocked out with rescue of CMV promoter driven mutant KIT) with VTP-50469 with or without WM-1119. FIG. 4D a bar plot showing a growth over time assay in GIST430, with relative cell count shown at day 42 following treatment with VTP-50469 at 0.5 μM with or without VTP-50469; the combination was used with each drug at 0.1 μM. FIG. 4E is a bar plot showing a cell cycle analysis showing the percentage of cells in G0/G1. S or G2/M comparing DMSO to 72-hour imatinib or 8 days of VTP-50469 at 0.5 μM with or without VTP-50469; the combination was used with each drug at 0.1 μM. FIG. 4F is a bar plot showing fold change compared to DMSO control of cells in early apoptosis or late apoptosis and cell death following 72 hour treatment with imatinib at 0.5 μM or 8 days of VTP-50469 at 0.5 μM or VTP-50469 with WM-1119. each drug at 0.1 μM.

FIGS. 5A-5W are a series of scatter and bar plots showing the transcriptional effects of Menin inhibition with and without MOZ inhibition. FIG. 5A is a scatter plot showing the ratio of expression between inhibitor and DMSO treatment for all expressed genes following 5 days of inhibitor treatment in GIST-T1 cells. FIG. 5B is a butterfly plot of all Hallmark gene sets indicating the NES and FDR q-value comparing VTP-50469 (blue) at day 5 to DMSO control. FIG. 5C is a scatter plot showing the Hallmark MTORC1 Signaling and EMT gene sets comparing DMSO and VTP-50469. FIG. 5D is a bar graph showing the relative expression. normalized to DMSO control. of all expressed genes. essential genes. genes downregulated >2.5-fold by 6 hours imatinib treatment and the Hallmark EMT gene set in GIST-T1 cells treated for 5 days with VTP-50469. FIG. 5E is a bar graph showing the relative expression. normalized to DMSO control. of all expressed genes comparing those with enriched Menin binding or those lacking enrichment. FIG. 5F is a bar graph showing the relative expression of core GIST TFs bound by Menin. FIGS. 5G and 5H are bar graphs showing the relative mRNA level by qRT-PCR of negative regulator of KIT signaling DUSP6. SE-associated NPR3 and essential gene USP1 in cells treated for 5 days with VTP-50469 at 0.5 μM or VTP-50469 with WM-1119. each drug at 0.1 μM. FIG. 5I is a heat map showing unsupervised hierarchical clustering of RNA-seq data comparing GIST-T1 with VTP-50469 treatment. FIG. 5J is a heat map showing unsupervised hierarchical clustering of RNA-seq data comparing GIST-T1/Cas9 cells transduced with sgRNAs. FIG. 5K is a heat map showing a Pearson correlation of control-normalized RNA-seq data. FIGS. 5L-5N are correlation plots of gene expression changes in the top 5.000 expressed transcripts comparing control-normalized sgRNAs or combination drug treatments. FIG. 5O is a heat map showing normalized enrichment scores (NES) from GSEA gene sets. FIGS. 5P-5S are GSEA plots showing changes in Menin/BRPF1-enriched genes. SE-associated genes, and HAND1-regulated genes. FIG. 5T is a box plot showing control-normalized expression of genes upregulated by HAND1. FIGS. 5U-5W are dot plots showing expression of select genes associated with GIST lineage. TFs. or HAND1 regulation across drug and sgRNA treatment conditions.

FIGS. 6A-6Q are a series of photographic images. scatter. bar. heatmaps. dot plots. and tracks that illustrate the alteration in protein interactions following Menin inhibition. FIG. 6A is a western blot of parental GIST-T1 cells or those following sgRNA deletion and rescue with a codon optimized MEAF6 construct fused to BirA* (R118G). FIG. 6B is a scatter plot of PSM and log₂ signal intensity of proximal proteins identified by MEAF6 BioID. FIG. 6C is a bar plot that shows the GO term enrichment for MEAF6 proximal proteins. FIG. 6D is a scatter plot of log₂ ratio of VTP-50469/DMSO signal intensity for MEAF6-enriched proteins following 2 days pre-treatment with inhibitors with an additional 24-hour treatment during biotin labeling. FIG. 6E is a heatmap showing unsupervised hierarchical clustering of DMSO-normalized signal intensity of 67 genes significantly changing in at least one condition in response to VTP-50469. or the combination treatment of VTP-50469 with WM-1119. FIGS. 6F-6G are dot plots of DMSO-normalized signal intensity for protein interactors enriched with VTP-50469 or VTP-50469 combined with WM-1119. FIG. 6H is a set of heat maps demonstrating spike-in normalized signal of DOT1L at MACS-defined peaks in GIST-T1 cells treated with DMSO or VTP-50469. FIGS. 6I and 6J are box plots showing spike-in normalized DOT1L (FIG. 6I) or MEAF6 (FIG. 6J) signal at MACS-defined peaks. FIG. 6K is a plot of tracks showing regions of genomic occupancy of spike-in normalized DOT1L under the indicated treatments. H3K79me2. MEAF6, and H3K27ac at the HAND1 locus. FIG. 6L shows a day 21 cell count normalized to DMSO following treatment of GIST-T1 or GIST48B, with the indicated concentrations of EPZ-5676. FIG. 6M is a heat map showing the Pearson correlation of control-normalized RNA-seq data from cells treated for 5 days with the indicated inhibitors FIG. 6N is a Pearson correlation of gene expression changes in expressed transcripts (n=5.000) comparing control-normalized drug treatments with EPZ-5676 and VTP-50469. FIG. 6O is GSEA plots showing changes in HAND1-regulated genes arising from EPZ-5676 treatment. FIGS. 6P and 6Q are dot plots showing expression of select genes associated with GIST lineage and TFs (n=4 per condition).

FIGS. 7A-7F are line plots and a series of photomicrographs that illustrate the effects of Menin inhibition on GIST in vivo. FIG. 7A is a line plot showing GIST-T1 cell line xenografts treated for 28 days with imatinib, VTP-50469, combination imatinib and VTP-50469, or vehicle control. FIG. 7B is a line plot showing PG27 PDX treated for 18 days with imatinib, VTP-50469, combination imatinib and VTP-50469, or vehicle control. FIG. 7C a series of photomicrographs showing tissue slices from PG27 tumors harvested at the end of the treatment period, fixed, sectioned, and stained with H&E. FIG. 7D is a line plot showing GIST-T1 cell line xenografts that were treated for 28 days. FIG. 7E is a heat map showing data from RNA-seq performed on GIST-T1 cell line xenografts treated for 5 or 10 days. FIG. 7F is a dot plot showing Expression in FPKM of select genes associated with GIST lineage, imatinib regulation, or cell proliferation.

FIGS. 8A-8N are a series of bar, line, and scatter plots illustrating the unique GIST dependencies. FIG. 8A is a bar plot showing the top 18 significantly enriched gene ontology terms among genes uniquely essential to GIST. FIG. 8B is a line plot of rank in screen and β-score highlighting Menin-MLL complex members. FIG. 8C is a line plot of rank in screen and β-score highlighting INO80 complex members. FIG. 8D is a line plot of rank in screen and β-score highlighting NuA4 histone acetyltransferase complex members. FIGS. 8E-8G are scatter plots of Ranked Sensitivity Scores from Project Drive cell lines for select members of the INOB0 and NuA4 complexes. FIG. 8H is a line plot of rank in screen and β-score highlighting FACT complex members. FIGS. 81-8J are scatter plots of Ranked Sensitivity Scores from Project Drive cell lines for members of the FACT complex. FIG. 8K is a line plot of rank in screen and β-score highlighting PAF1 complex members. FIGS. 8L-8M are scatter plots of Ranked Sensitivity Scores from Project Drive cell lines for select members of the PAF1 complex. FIG. 8N is a bar plot showing relative reads for the top 8 sgRNAs targeting the indicated genes in GIST-T1 or GIST430, normalized to baseline plasmid library (n=2 per sgRNA).

FIGS. 9A-9E are a series of line and scatter plots that illustrate the PCR2 complex dependency in GIST. FIG. 9A is a line plot showing a plot of rank in screen and β-score highlighting core PRC2 complex members. FIGS. 9B-9C are Ranked Sensitivity Scores from Project Drive cell lines for select members of the PRC2 complex. FIG. 9D is a plot of rank and CERES dependency score for EZH2 complex members across DepMap cell lines (n=726), with the dotted line at-1 indicating significant dependency. FIG. 9E shows the top gene dependency correlations of EZH2 in DepMap. Co-dependent chromatin modifying enzymes and complex members are labeled.

FIGS. 10A-10H are a series of diagrams and tracks that illustrates the Menin-MLL complex localizations in GIST. FIG. 10A is a diagram showing the overlap in enriched regions between Menin and BRPF1, with select GIST-associated genes indicated. FIGS. 10B-10H are tracks showing regions of genomic occupancy of the TF HAND1, Menin-MLL complex members Menin and MLL In, and histone marks H3K4me3, H3K9ac and H3K27ac; FIG. 10B shows the OSR1 loci. FIG. 10C shows the PDGFRA loci; FIG. 10D shows the KIT loci, FIG. 10E shows the KDR loci, FIG. 10F shows the MEIS1 loci, FIG. 10G shows the HAND1 loci. and FIG. 10H shows the NPR3 loci.

FIG. 11 is a bar plot that shows a DMSO-normalized cell count after the first passage of slowly growing GIST cell lines GIST430 and GIST882 inhibited with VTP-50469 or VTP-50469 combined with WM-1119.

FIGS. 12A-12C are a series of scatter and bar plots that illustrate the transcriptional effects of Menin inhibition. FIG. 12A is a scatter plot showing the ratio of expression between inhibitor and DMSO treatment for the top 500 essential genes following 5 days of inhibitor treatment. FIG. 12B is a bar plot showing the relative expression of negative regulators of KIT signaling SPRY2, SPRY4 and DUSP6 upon 1- or 5-day treatment with VTP-50469. FIG. 12C is a bar plot showing the relative expression of KIT upon 1- or 5-day treatment with VTP-50469.

FIGS. 13A-13J are a series of heat maps, scatter plots, and tracks that illustrate the results of ChIP-seq of DOT1L, H3K79me2, and MEAF6 and the effects of VTP-50469. FIG. 13A is heat maps demonstrating spike-in normalized signal of MEAF6 at MACS-defined peaks in GIST-T1 cells treated with DMSO or VTP-50469. FIGS. 13B-13C are scatter plots showing enriched genomic regions of DOT1L, and H3K79me2 binding. FIG. 13D is a series of heat maps demonstrating genomic localization in GIST-T1 of DOT1L, H3K79me2 and MEAF6 by ChIP-seq. FIG. 13E is a set of tracks that shows regions of genomic occupancy of spike-in normalized DOT1L under the indicated treatments, H3K79me2, and H3K27ac at the GPR20 locus. FIG. 13F is a dot plot showing the top 70 gene dependency correlations of DOT1L in DepMap, with members of Menin-MLL, MOZ and PRC2 complex indicated. FIG. 13G is a box plot showing DMSO-normalized signal for DOT1L in regions with enriched (n=1,343) or typical (n=45,256) signal for DOT1L. FIG. 13H is photographs of a Western blot showing DOT1L signal following 5 days of treatment with the indicated inhibitors. FIG. 13I is a dot plot showing levels of DOT1L expression by RNA-seq following 5 days of treatment with the indicated drugs. FIG. 13J is a bar plot showing day 21 GIST-T1 cell count in a growth over time assay comparing sgRNAs targeting two DOT1L exons or Luc or RPS19 as control.

FIGS. 14A-14F are line plots and a series of photomicrographs that show the effects of Menin inhibition in vivo. FIG. 14A is a line plot showing the weight of mice engrafted with the GIST-T1 cell line and treated for 28 days with imatinib, VTP-50469, a combination of imatinib and VTP-50469, and a vehicle control. FIG. 14B is a line plot showing the weight of mice engrafted with the PG27 PDX and treated for 18 days with imatinib, VTP-50469, a combination of imatinib and VTP-50469, and a vehicle control. FIG. 14C a series of photomicrographs showing tissue slices from PG27 tumors were harvested at the end of the treatment period and fixed tissues sectioned and evaluated for Ki-67 (top row) and cleaved caspase-3 (bottom row): scale bar=25 μm. FIG. 14D is a line plot showing tumor size after mice were engrafted with GIST-T1 cell lines and treated with sgRNAs. FIG. 14E is a line plot showing the weight of mice engrafted with the GIST-T1 cell line and treated for 28 days with VTP-50469, WM-1119, a combination of VTP-50469 and WM-1119, or a vehicle control. FIG. 14F is a box plot showing Control-normalized expression of all expressed genes (n=7,434) or those whose expression is upregulated by HAND1 (n=438) in each treatment group.

FIGS. 15A-C is a set of line and bar plots showing KAT6A, Menin and BRPF1 inhibition in GIST cell lines. FIG. 15A is a line plot that shows growth over time assay following treatment of GIST-T1 or GIST48B with 50 nM imatinib. FIG. 15B is a bar plot that shows DMSO-normalized cell count after the first passage of slowly growing GIST cell lines GIST430 (day 6), GIST882 (day 12) and GIST48 (day 12) in comparison to GIST-T1 (day 4). FIG. 15C is a line plot that shows growth over time assay treating GIST-T1 or GIST48B cells with selective BRPF1 inhibitors GSK6853 or PFI-4.

FIGS. 16A-16H is a set of bar plots, box plots and heat maps showing the Transcriptional effects of MOZ and Menin disruption. FIG. 16A is a heat map of control normalized expression of 10 GIST-associated TFs in response to drug or sgRNA treatment. FIG. 16B is a box plot that shows DMSO-normalized expression of 18 GIST-associated TFs in the indicated drug treatment (n=4 per condition). FIGS. 16C-16F is a set of bar plots that show relative mRNA level of negative regulator of KIT signaling DUSP6 and HAND1- and SE-associated gene NPR3 in GIST cell lines. FIG. 16G is a heat map that shows GSEA data indicating the NES of Reactome translation-associated gene sets in each drug or sgRNA treatment condition. FIG. 16H is a box plot that shows control-normalized expression of all translation-associated genes (n=48) in each sgRNA and drug treatment condition.

### DETAILED DESCRIPTION OF THE DISCLOSURE

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

By “agent” is meant any small compound, antibody, nucleic acid molecule, or polypeptide, or fragments thereof. Unless specifically stated or obvious from context, as used herein, the term “or” is understood to be inclusive. Unless specifically stated or obvious from context, as used herein, the terms “a”, “an”, and “the” are understood to be singular or plural.

Any compositions or methods provided herein can be combined with one or more of any of the other compositions and methods provided herein.

The transitional term “comprising.” which is synonymous with “including.” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below:

### Methods of Use

In some aspects, the present disclosure is directed to treating GIST in a subject. The method entails administering to a subject in need thereof an effective amount or a therapeutically effective amount of a Menin inhibitor.

GIST is a soft tissue sarcoma often characterized by recurrent activating mutations in or around the tyrosine kinase KIT gene and/or PDGFRA gene. The phrase “in or around” as used herein refers to a mutation within the coding region of a gene or a mutation in a 5′ or 3′ proximal region of the gene that contributes to gene function (e.g., a regulatory region that affects gene transcription). GIST both lacks oncogene amplification and relies upon an established network of transcription factors. Unique chromatin modifying enzymes are shown in the working examples as essential in orchestrating the GIST epigenome: for example, KMT2A/MLL1 is established herein as a previously unknown dependency of GIST, and, more broadly, is found to exhibit similar regulation across select cancer subtypes. KMT2A/MLL1 is a member of the Menin-MLL complex and responsible for H3K4 methylation and transcriptional activation (Ruthenburg et al., Molecular Cell 25:15-30 (2007), Krivtsov et al., Nat. Rev. Cancer 7:823-33 (2007)). In some embodiments, the subject has been diagnosed with GIST that has a mutation in or around the KIT gene. In some embodiments, the mutation is an activating mutation. Activating mutations cause the mutated protein to remain in a dysregulated state as compared to an unmutated protein. Activating mutations in kinase domains most often lead to ligand-independent activation of the kinase domain and therefore target phosphory lation. In some embodiments, the subject has a metastatic GIST.

The term “subject” (or “patient”) as used herein includes all members of the animal kingdom prone (or disposed) to or suffering from GIST. In some embodiments, the subject is a human. Therefore, a subject “having GIST,” or “in need of” treatment according to the present disclosure broadly embraces subjects who have been positively diagnosed, including subjects having active disease who may have been previously treated with one or more rounds of therapy, and subjects who are not currently being treated (e.g., in remission) but who might still be at risk of relapse, and subjects who have not been positively diagnosed but who are predisposed to cancer or autoimmune disease (e.g., on account of the basis of prior medical history and/or family medical history, or who otherwise present with a one or more risk factors such that a medical professional might reasonably suspect that the subject was predisposed to GIST).

The terms “treat”, “treating”, and “treatment” as used herein refer to any type of intervention, process performed on, or the administration of the effective amount or the therapeutically effective amounts of the Menin inhibitor, TKI, and/or MOZ inhibitor to the subject in need thereof with the therapeutic objective (“therapeutic effect”) of reversing, alleviating, ameliorating, inhibiting, diminishing, slowing down, arresting, stabilizing, or preventing the onset, progression, development, severity or recurrence of a symptom, complication or condition, or biochemical indicia associated with GIST.

Active Agents employed in the practice of the present disclosure are Menin inhibitors. As disclosed herein, in some embodiments, one or more additional active agents may be employed, including inhibitors of tyrosine kinase (TKI) and inhibitors of MOZ (monocytic leukemic zinc-finger: also known as lysine (K) acetyltranferase 6A (KAT6A), which is a histone acetyltransferase (HAT)).

The term “inhibitor” is used in its broadest sense and includes any agent such as a small molecule, nucleic acid (e.g., ribozyme, antisense nucleic acid, siRNA), antibody or functional fragment thereof, peptide, peptidomimetic or aptamer, that acts to disrupt, directly or indirectly, and reduce or even eliminate the function of the target.

### Menin Inhibitors

The terms “Menin inhibitor”, “Menin inhibitors” and “Menin-MLL complex inhibitors” are used herein interchangeably and may be understood in their broadest sense. A Menin inhibitor includes one or a combination of any agents such as a small molecule, nucleic acid (e.g., siRNAs), or antibody, peptide, peptidomimetic or aptamer that acts to disrupt that acts to disrupt, directly or indirectly, and reduce or even eliminate the function or expression of the Menin protein, the multiple endocrine neoplasia 1 (MEN1) gene, or the Menin-MLL complex. Protein disruption may include direct activity blockage, protein-protein interaction blocking, or the like. Menin, the protein product of the MEN1 (multiple endocrine neoplasia syndrome type 1) gene, interacts with mixed lineage leukemia (MLL) family proteins in a histone methyltransferase complex including MLL1 (also known as lysine (K)-specific methyltransferase 2A (KMT2A)), Ash2, Rbbp5, and WDR5. As a consequence of chromosomal rearrangements of the MLL gene, MLL is fused with one of over 60 different protein partners, resulting in upregulated expression of HOXA9 and MEIS1 genes that are critical to leukemogenesis. Unlike AML or ALL, MLL fusion proteins do not occur in GIST.

Representative small molecule Menin inhibitors include VTP-50469 (5-fluoro-N,N-diisopropyl-2-((4-(7-(((1r,4r)-4-(methylsulfonamido)cyclohexyl)methyl)-2,7-diazaspiro[3.5]nonan-2-yl)pyrimidin-5-yl)oxy)benzamide), KO-539 ((R)-4-methyl-5-((4-((2-(methylamino)-6-(2,2,2-trifluoroethyl) thieno[2,3-d]pyrimidin-4-yl)amino) piperidin-1-yl)methyl)-1-(2-(4-(methylsulfonyl) piperazin-1-yl) propyl)-1H-indole-2-carbonitrile, also used in NCT04067336), JNJ-75276617 ((R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide, also used in NCT04811560), SNDX-5613 (N-ethyl-2-((4-(7-(((1r,4r)-4-(ethylsulfonamido)cyclohexyl)methyl)-2,7-diazaspiro[3.5]nonan-2-yl)pyrimidin-5-yl)oxy)-5-fluoro-N-isopropylbenzamide, also used in NCT04065399), DS-1594 ((1R,2S,4R)-4-((4-(5,6-dimethoxypyridazin-3-yl)benzyl)amino)-2-(methyl (6-(2,2,2-trifluoroethyl) thieno[2,3-d]pyrimidin-4-yl)amino)cyclopentan-1-ol, also used in NCT04752163), BMF-219 ((R)—N-(1-(2-(2-((4-(4-morpholino-7H-pyrrolo[2,3-d]pyrimidin-6-yl)phenyl)amino)-2-oxoethyl) pyridin-4-yl) piperidin-3-yl) but-2-ynamide), DSP-5336 (N-ethyl-5-fluoro-N-isopropyl-2-((5-(7-((1S,3S,4R)-5-methylene-2-azabicyclo[2.2.2]octane-3-carbonyl)-2,7-diazaspiro[3.5]nonan-2-yl)-1,2,4-triazin-6-yl)oxy)benzamide, also used in NCT04988555), the antibody A300-105A (commercially available from Bethyl Laboratories), MI-3453 (N-(3-((2-cyano-4-methyl-5-((4-((2-(methylamino)-6-(2,2,2-trifluoroethyl) thieno[2,3-d]pyrimidin-4-yl)amino) piperidin-1-yl)methyl)-1H-indol-1-yl)methyl) bicyclo[1.1.1]pentan-1-yl) formamide, M-808 (methyl((1S,2R)-2-((S)-2-(azetidin-1-yl)-1-(3-fluorophenyl)-1-(1-((1-(4-((1-((E)-4-(piperidin-1-yl) but-2-enoyl) azetidin-3-yl) sulfonyl)phenyl) azetidin-3-yl)methyl) piperidin-4-yl)ethyl)cyclopentyl) carbamate), MI-0202 (4-(4-(5,5-dimethyl-4,5-dihydrothiazol-2-yl) piperazin-1-yl)-6-(2,2,2-trifluoroethyl) thieno[2,3-d]pyrimidine), MI-503 (1-((1H-pyrazol-4-yl)methyl)-4-methyl-5-((4-((6-(2,2,2-trifluoroethyl) thieno[2,3-d]pyrimidin-4-yl)amino) piperidin-1-yl)methyl)-1H-indole-2-carbonitrile), MI-463 (4-methyl-5-((4-((6-(2,2,2-trifluoroethyl) thieno[2,3-d]pyrimidin-4-yl)amino) piperidin-1-yl)methyl)-1H-indole-2-carbonitrile), MI-136 (5-((4-((6-(2,2,2-trifluoroethyl) thieno[2,3-d]pyrimidin-4-yl)amino) piperidin-1-yl)methyl)-1H-indole-2-carbonitrile), and ML-227 (4-(3-(4-(cyclopentyl(hydroxy) (phenyl)methyl) piperidin-1-yl) propoxy)benzonitrile). The structures of these small molecule inhibitors are as follows:

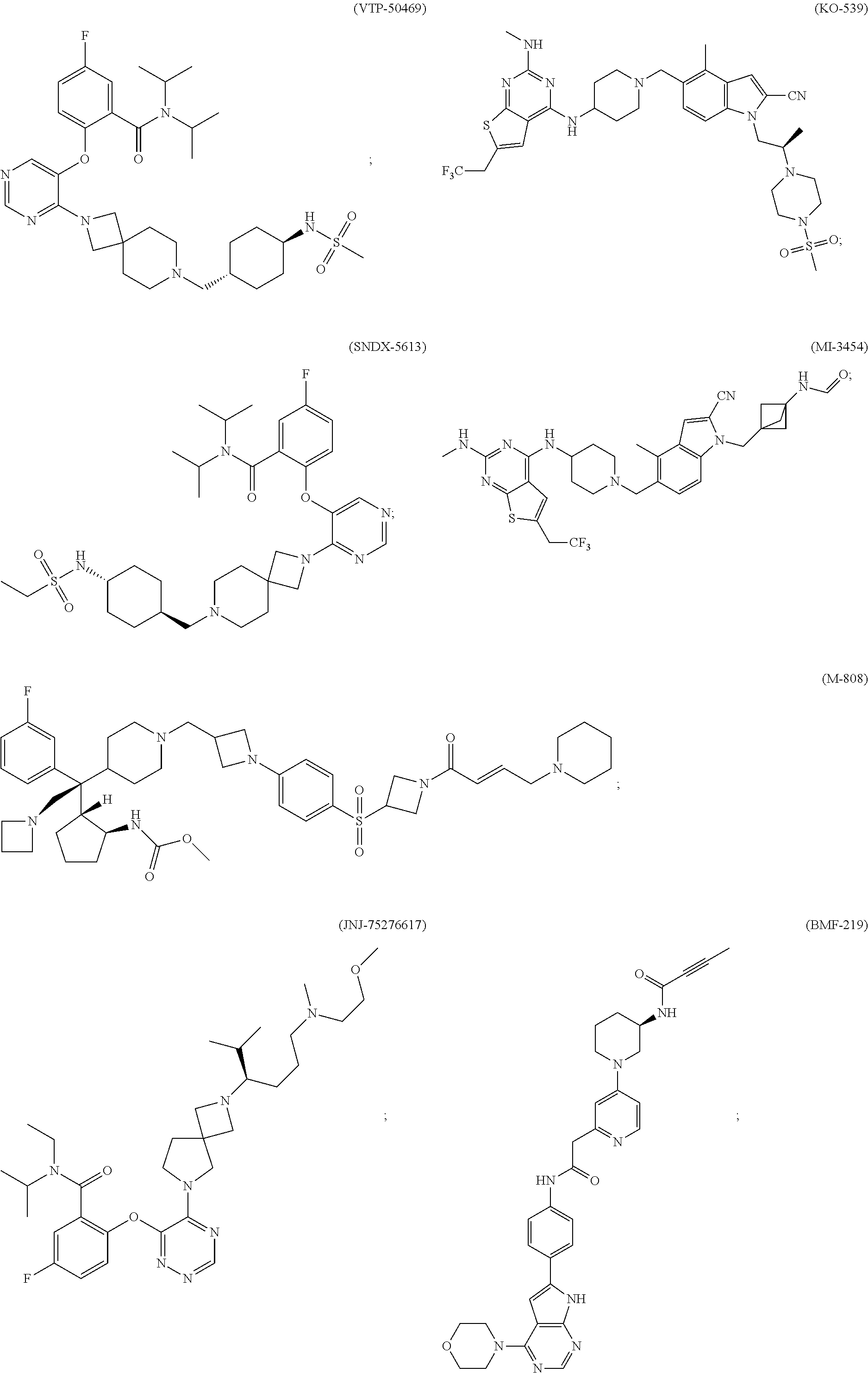

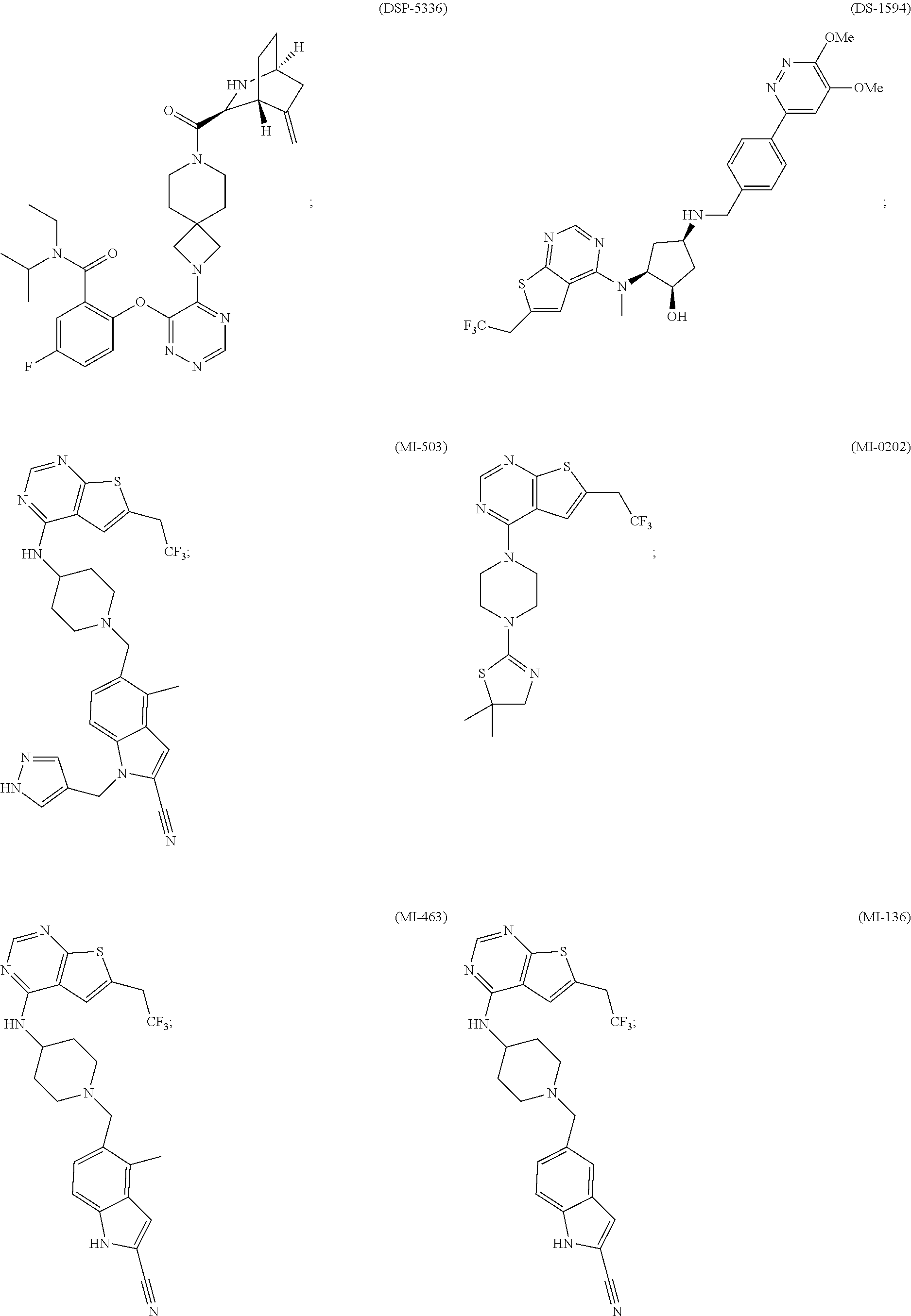 [SMILES: C=C1C[C@@H]2CC[C@H]1[C@@H](C(=O)N1CCC3(CC1)CN(c1ncnnc1Oc1ccc(F)cc1C(=O)N(CC)C(C)C)C3)N2 ; COc1cc(-c2ccc(CN[C@H]3C[C@@H](O)[C@@H](N(C)c4ncnc5sc(CC(F)(F)F)cc45)C3)cc2)nnc1OC ; Cc1c(CN2CCC(Nc3ncnc4sc(CC(F)(F)F)cc34)CC2)ccc2c1cc(C#N)n2Cc1cn[nH]c1 ; CC1(C)CN=C(N2CCN(c3ncnc4sc(CC(F)(F)F)cc34)CC2)S1 ; Cc1c(CN2CCC(Nc3ncnc4sc(CC(F)(F)F)cc34)CC2)ccc2[nH]c(C#N)cc12 ; N#Cc1cc2cc(CN3CCC(Nc4ncnc5sc(CC(F)(F)F)cc45)CC3)ccc2[nH]1]

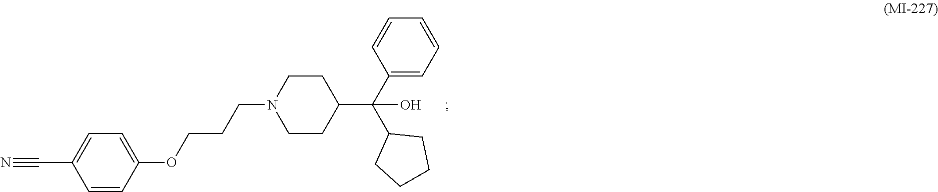 [SMILES: N#Cc1ccc(OCCCN2CCC(C(O)(c3ccccc3)C3CCCC3)CC2)cc1]

Other Menin inhibitors that may be useful in the practice of the present disclosure are known in the art. See. e.g., WO 2017/112768, WO 2017/214367, WO 2018/053267, WO 2020/069027 A1, WO 2021/207335 A1. U.S. 2021/0115018 A1. U.S. 2019/0307750. U.S. 20160339035 (compounds of formula (I) therein), and Borkin et al., Cancer Cell 27 (4): 589-602 (2015).

Further Menin inhibitors that may be useful in the practice of the present disclosure include MI-2-2, which inhibits the interaction between Menin and an MLL Grembecka et al., Nat. Chem. Biol. 8:277-284 (2012); Shi et al., Blood 120:4461-4469 (2102)), and N,N′-bis(4-aminophenyl)-N,N′-dimethylethylenediamine (also known as ISC-30, and which inhibits the interaction of an MLL enzyme and menin), and Krivtsov et al., Cancer Cell. 36 (6): 660-673 (2019), Klossowski et al., J. Clin. Invest. 130:981-97 (2020), Xu et al., J. Med. Chem. 63:4997-5010 (2020).

In some embodiments, the Menin inhibitor is an interfering RNA, for example, a short interfering RNA (siRNA), used as active agent to decrease the level of MEN1 or the level of another Menin-MLL complex member. RNA interference (RNAi) is a phenomenon in which the introduction of double-stranded RNA (dsRNA) into a diverse range of organisms and cell types causes degradation of the complementary mRNA. Soutschek et al., 432:173-178 (2004) describe a chemical modification to siRNAs that aids in intravenous systemic delivery. Optimizing siRNAs involves consideration of overall G/C content, C/T content at the termini, melting temperature (Tm) and the nucleotide content of the 3′ overhang. See, for instance, Schwartz et al., Cell 115:199-208 (2003) and Khvorova et al., Cell 115:209-216 (2003). Therefore, the present disclosure also includes methods of decreasing levels of MEN1, MOZ, or other target protein using RNAi technology. Nucleic acid sequences of representative siRNAs that bind to a member of the Menin-MLL complex are set forth in Table 1.

TABLE 1
Nucleic Acid sequences of Menin-MLL
complex-binding siRNAs

| Oligonucleotide | Sequence (5′-->3′) |
|---|---|
| sgKMT2A_A_For | CACCGAGGGGTCTTAATGATCCGCG |
| | (SEQ ID NO: 1) |
| sgKMT2A_A_Rev | AAACCGCGGATCATTAAGACCCCTC |
| | (SEQ ID NO: 2) |
| sgKMT2A_B_For | CACCGGATCCTTCTACTTGCATGGG |
| | (SEQ ID NO: 3) |
| sgKMT2A_B_Rev | AAACCCCATGCAAGTAGAAGGATCC |
| | (SEQ ID NO: 4) |
| sgMEN1_A_For | CACCGACGTCGTCGATGGAGCGCAG |
| | (SEQ ID NO: 5) |
| sgMEN1_A_Rev | AAACCTGCGCTCCATCGACGACGTC |
| | (SEQ ID NO: 6) |
| sgMEN1_B_For | CACCGAGAGGGCGGCGATGATAGAC |
| | (SEQ ID NO: 7) |
| sgMENI_B_Rev | AAACGTCTATCATCGCCGCCCTCT |
| | (SEQ ID NO: 8) |

The Menin inhibitor may be administered to a patient as a monotherapy or by way of combination therapy (e.g., in combination with a TKI and/or a MOZ inhibitor). Both mono- and combination therapies may be “front/first-line”, i.e., as an initial treatment in patients who have undergone no prior anti-GIST cancer treatment regimens, either alone or in combination with other treatments: or “second-line”, as a treatment in patients who have undergone a prior anti-cancer treatment regimen, either alone or in combination with other treatments: or as “third-line”, “fourth-line”, etc. treatments, either alone or in combination with other treatments. Therapy may also be given to patients who have had previous treatments which were unsuccessful or partially successful but who became intolerant to the particular treatment. Therapy may also be given as an adjuvant treatment, i.e., to prevent reoccurrence of GIST in patients with no currently detectable disease or after surgical removal of a tumor. Thus, in some embodiments, the inhibitor(s) may be administered to a patient who has received another therapy, such as chemotherapy, radioimmunotherapy, surgical therapy, immunotherapy, radiation therapy, targeted therapy, or any combination thereof.

Combination Therapy with Tyrosine Kinase Inhibitors (TKI) and/or MOZ inhibitors

In some embodiments, the subject is treated by way of Menin inhibitor therapy in combination or concurrently with an effective amount or a therapeutically effective amount of a TKI and/or a MOZ inhibitor. Blocking KIT, or MOZ may provide an additional means of enhancing the therapeutic effect of the Menin inhibitor.

The terms “in combination” and “concurrently” as used in the context of combination therapy mean that the active agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second inhibitor, the first inhibitor is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

### Tyrosine Kinase Inhibitors

TKIs includes any one or a combination of agents such as a small molecule, nucleic acid (e.g., siRNAs), or antibody, peptide, peptidomimetic or aptamer that acts to disrupt that acts to disrupt, directly or indirectly, and reduce or even eliminate the function or expression of the KIT protein, or KIT. In some embodiments, the TKI is imatinib, sunitinib, regorafenib, avapritinib, ripretinib, or nilotinib. The TKI may be an antibody, for example, the anti-KIT antibodies monoclonal anti-D4 and anti-D5. See, Shi et al., Proc. Natl. Acad. Sci. USA 113 (33):E4784-93 (2016). In some embodiments, the KIT inhibitor is an antibody fragment, for example, the bivalent antibody fragments 2D1-Fc and 3G1-Fc. See, Gall et al., Mol. Cancer. Ther. 14 (11): 2595-605 (2015). Combinations of two or more TKI inhibitors may be used.

In some embodiments, the TKI is administered subsequent to administration of the Menin inhibitor. In some embodiments, the TKI is administered substantially simultaneously with administration of the Menin inhibitor (i.e., concurrently). In some embodiments, the TKI is administered prior to administration of the Menin inhibitor.

### MOZ Inhibitors

In some embodiments, the additional active agent may be an effective amount of a MOZ inhibitor. In some embodiments, the additional active agent may be a therapeutically effective amount of a MOZ inhibitor. A MOZ inhibitor includes one or a combination of any agents such as a small molecule, nucleic acid (e.g., siRNAs), or antibody, peptide, peptidomimetic or aptamer that acts to disrupt that acts to disrupt, directly or indirectly, and reduce or even eliminate the function or expression of the MOZ protein or the MOZ gene. In some embodiments, the MOZ inhibitor is administered subsequent to administration of the Menin inhibitor. In some embodiments, the MOZ inhibitor is administered substantially simultaneously with administration of the Menin inhibitor. In some embodiments, the Menin inhibitor is combined with both a TKI and a MOZ inhibitor. In some embodiments, the TKI is administered subsequent to administration of the MOZ inhibitor. In some embodiments, TKI is administered substantially simultaneously with administration of the MOZ inhibitor.

Representative examples of MOZ inhibitors that may be useful in the practice of the present disclosure include WM-1119 (2-fluoro-N′-(3-fluoro-5-(pyridin-2-yl)benzoyl)benzenesulfonohydrazide), WM-8014 (N′-(4-fluoro-5-methyl-[1, l′-biphenyl]-3-carbonyl)benzenesulfonohydrazide), PF-9363 (N′-(4-fluoro-5-methyl-[1, l′-biphenyl]-3-carbonyl)benzenesulfonohydrazide), and the antibody 21620002 (commercially available from Novus Biologicals).

The structures of these representative small molecule MOZ inhibitors are as follows:

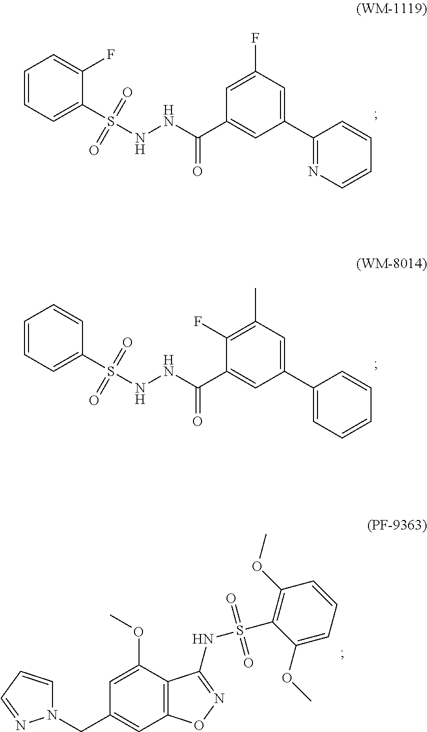 [SMILES: O=C(NNS(=O)(=O)c1ccccc1F)c1cc(F)cc(-c2ccccn2)c1 ; Cc1cc(-c2ccccc2)cc(C(=O)NNS(=O)(=O)c2ccccc2)c1F ; COc1cccc(OC)c1S(=O)(=O)Nc1noc2cc(Cn3cccn3)cc(OC)c12]

In some embodiments, the MOZ inhibitor is interfering RNA (e.g., a siRNA) used as active agent to decrease the level of MOZ or the level of another MOZ complex member. Nucleic acid sequences of representative siRNAs that bind to a member of the MOZ complex are set forth in Table 2.

TABLE 2
Nucleic Acid sequences of MOZ
complex-binding siRNAs

| Oligonucleotide | Sequence (5′-->3′) |
|---|---|
| sgBRPF1_A_For | CACCGGCTTAGTCGCCCGCAAGTTG |
| | (SEQ ID NO: 9) |
| sgBRPF1_A_Rev | AAACCAACTTGCGGGCGACTAAGCC |
| | (SEQ ID NO: 10) |
| sgBRPF1_B_For | CACCGAGGCGACCCTAATGCGCTAG |
| | (SEQ ID NO: 11) |
| sgBRPF1_B_Rev | AAACCTAGCGCATTAGGGTCGCCTC |
| | (SEQ ID NO: 12) |
| sgKAT6A_A_For | CACCGTGATAGCCAATCGTAACTGC |
| | (SEQ ID NO: 13) |
| sgKAT6A_A_Rev | AAACGCAGTTACGATTGGCTATCAC |
| | (SEQ ID NO: 14) |
| sgKAT6A_B_For | CACCGTCACTCGAACCGTTAGTTCA |
| | (SEQ ID NO: 15) |
| sgKAT6A_B_Rev | AAACTGAACTAACGGTTCGAGTGAC |
| | (SEQ ID NO: 16) |
| sgMEAF6_A_For | CACCGCATGGCGATGCACAACAAGG |
| | (SEQ ID NO: 17) |
| sgMEAF6_A_Rev | AAACCCTTGTTGTGCATCGCCATGC |
| | (SEQ ID NO: 18) |
| sgMEAF6_B_For | CACCGGCGGCTCTTCAGTAAATCCT |
| | (SEQ ID NO: 19) |
| sgMEAF6_B_Rev | AAACAGGATTTACTGAAGAGCCGCC |
| | (SEQ ID NO: 20 16) |

In some embodiments, the MOZ inhibitor is administered subsequent to administration of the Menin inhibitor. In some embodiments, the MOZ inhibitor is administered substantially simultaneously with administration of the Menin inhibitor (i.e., concurrently). In some embodiments, the MOZ inhibitor is administered prior to administration of the Menin inhibitor.

With respect to embodiments that entail administration of a TKI and a MOZ inhibitor (in addition to the Menin inhibitor), the MOZ inhibitor may be administered prior to, substantially simultaneously, or subsequent to administration of the TKI.

In some embodiments, the MOZ inhibitor is administered subsequent to administration of the TKI and the Menin inhibitor. In some embodiments, the MOZ inhibitor is administered substantially simultaneously with administration of the TKI and the Menin inhibitor (i.e., concurrently). In some embodiments, the Menin inhibitor is administered subsequent to administration of the MOZ inhibitor and the TKI.

### Compositions and Formulations

The active agents described herein may be formulated into pharmaceutical compositions in accordance with known techniques. Pharmaceutical compositions of the disclosure include an effective amount of a Menin inhibitor, alone or in combination with effective amounts of TKIs, and MOZ inhibitors. In some embodiments, pharmaceutical compositions of the disclosure include an effective amount or a therapeutically effective amount of a Menin inhibitor, alone or in combination with effective amounts or therapeutically effective amounts of TKIs, and MOZ inhibitors. The active agent(s) may be in the form of a pharmaceutically acceptable salt, or an isomer (e.g., stereoisomers) thereof. Salts and stereoisomers are embraced by the terms “inhibitor(s)” and “active agent(s)”. As used herein, a “pharmaceutically acceptable salt” means any non-toxic salt that, upon administration to a recipient, is capable of providing, either directly or indirectly, a compound or a prodrug of a compound of this disclosure. Pharmaceutically acceptable salts may be formed with acids, representative examples of which include hydrochloric, sulfuric, acetic, lactic, tartaric, malic, and succinic acids.

The active agents disclosed herein and their pharmaceutically acceptable salts and stereoisomers may be formulated individually or together, in combinations of two or more, into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the inhibitor may be administered relatively quickly such as in the case of a single-dose treatment and/or an acute condition.

In some embodiments, the active agents are formulated for oral or intravenous administration (e.g., systemic intravenous injection).

Accordingly, active agents may be formulated into solid compositions (e.g., powders, tablets, dispersible granules, capsules, cachets, and suppositories), liquid compositions (e.g., solutions in which the inhibitor is dissolved, suspensions in which solid particles of the inhibitor are dispersed, emulsions, and solutions containing liposomes, micelles, or nanoparticles, syrups and elixirs), semi-solid compositions (e.g., gels, suspensions and creams), and gases (e.g., propellants for aerosol compositions). Inhibitors may also be formulated for rapid, intermediate or extended release.

Solid dosage forms for oral administration include capsules, tablets, pills, powders, and granules. In such solid dosage forms, the active inhibitor is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings and may further contain an opacifying agent.

In some embodiments, inhibitors of the present disclosure may be formulated in a hard or soft capsule, such as a gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups, and elixirs. In addition to the inhibitor, the liquid dosage forms may contain an aqueous or non-aqueous carrier (depending upon the solubility of the inhibitor) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipient, representative examples of which include wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P, and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the inhibitor from a parenterally administered formulation may also be accomplished by suspending the inhibitor in an oily vehicle.

### Dosage Amounts

As used herein, the terms, “effective amount” and “therapeutically effective amount” refers to an amount of an active agent disclosed herein (e.g., a Menin inhibitor, a TKI, or a MOZ inhibitor) or a pharmaceutically acceptable salt or isomer thereof, effective in producing the desired response in a GIST patient. Therefore, the terms “effective amount” and “therapeutically effective amount” embraces amounts of active agents, that when administered, induces a positive modification in the GIST, or is sufficient to inhibit development or progression of GIST, or alleviate to some extent, one or more of the symptoms of GIST, or which simply kills or inhibits the growth of GIST or otherwise blocks or reduces the activity of the Menin-MLL complex in diseased cells. The effective amount of an active agent may vary depending on several factors among which may include the severity and stage of GIST, the mode of administration, the age, body weight, and general health of the subject, and like factors well known in the medical arts. See, for example, Goodman and Gilman's. The Pharmacological Basis of Therapeutics, 10th Edition, A. Gilman, J. Hardman and L. Limbird, eds., McGraw-Hill Press, 155-173, 2001. Ultimately, an attending physician or veterinarian will decide upon the appropriate amount and dosage regimen.

Active agents useful in the practice of the present disclosure may be effective over a wide dosage range. In some embodiments, the total daily dosage of a given active agent (e.g., for adult humans) may range from about 0.001 to about 1600 mg, from 0.01 to about 1600 mg, from 0.01 to about 500 mg, from about 0.01 to about 100 mg. from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the active agent is administered per day. By way of example, capsules may be formulated with from about 1 to about 200 mg of an active agent (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the active agent is administered per day.

In some embodiments, suitable daily dosages of a Menin inhibitor may range from 1 ng/kg to about 200 mg/kg, about 1 μg/kg to about 100 mg/kg, or about 1 mg/kg to about 50 mg/kg of body weight. Other dosage amounts of Menin inhibitors are disclosed in the art. See. e.g., International Application Publications WO 2017/112768, WO 2017/214367, WO 2018/053267, WO 2020/069027 A1, WO 2021/207335 A1, and U.S. Patent Application Publications 2021/0115018 A1, and 2019/0307750.

In some embodiments, the daily dosage of the TKI imatinib is about 100 mg/day. In some embodiments, the KIT inhibitor is administered in a daily dosage of about 300 mg/day, about 340 mg/day, about 400 mg/day, about 600 mg/day, or about 800 mg/day.

In some embodiments, the daily dosage of the TKI sunitinib is about 50 mg e.g., orally once daily for 4 weeks followed by 2 weeks of no treatment, typically in the form of hard gelatin capsules containing 12.5, 25, or 50 mg of sunitinib.

In some embodiments, the daily dosage of the TKI regorafenib is about 160 mg (e.g., orally, for 21 days followed by one week off, typically in the form of 40 mg-film coated tablets.

In some embodiments, the daily dosage of the TKI avapritinib is about 300 mg (e.g., orally once daily, typically in the form of film-coated capsules containing 25, 50 100, 200, or 300 mg.

In some embodiments, the daily dosage of the TKI ripretinib is about 150 mg (e.g., orally once daily, typically in the form of 50 mg tablets).

In some embodiments, the daily dosage of the TKI nilotinib is about 300-400 mg (e.g., 150 and 200 mg hard capsules, typically taken twice daily at approximately 12-hour intervals on an empty stomach).

In some embodiments, the daily dosage of a MOZ inhibitor may range from about 0.5 μg to about 50 mg per kilogram of body weight of the subject. In some embodiments, the dosage of the MOZ inhibitor may range from about 1 μg to about 10 mg per kilogram of body weight of the subject, and in some embodiments, from about 3 μg to about 1 mg per kilogram of body weight of the subject.

The methods may entail administration of a Menin inhibitor, and optionally one or more additional active agents or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days) followed by a 7-day “off” period, or administration for 4 weeks followed by a 14-day “off” period. In other embodiments, the active agent(s) s may be dosed twice a day (BID) over the course of two and a half days (for a total of 5 doses) or once a day (QD) over the course of two days (for a total of 2 doses). In other embodiments, the active agent(s) may be dosed once a day (QD) over the course of five days.

### Additional Combination Therapies

The present methods may entail administration of at least one other active, anti-cancer agent. Representative anti-cancer agents are disclosed in U.S. Pat. No. 9,101,622 (Section 5.2 thereof).

Yet other therapies include immunotherapy, chemotherapy and radiation.

Immunotherapy, including immune checkpoint inhibitors may be employed to treat a diagnosed cancer. Immune checkpoint molecules include, for example, PD1, CTLA4, KIR, TIGIT, TIM-3, LAG-3, BTLA, VISTA, CD47, and NKG2A. Clinically available examples of immune checkpoint inhibitors include durvalumab (ImfinziR), atezolizumab (Tecentriq®), and avelumab (BavencioR). Clinically available examples of PD1 inhibitors include nivolumab (Opdivo R), pembrolizumab (Keytruda®), and cemiplimab (Libtayo R).

Combination chemotherapies include, for example, AbraxaneR, altretamine, docetaxel, Herceptin R, methotrexate, Novantrone R, Zoladex R, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, TaxolR, gemcitabien, NavelbineR, farnesyl-protein tansferase inhibitors, transplatinum, 5-fluorouracil, vincristine, vinblastine and methotrexate, or any analog or derivative variant of the foregoing and also combinations thereof.

Combination radiotherapies include what are commonly known as gamma-rays, X-rays, and/or the directed delivery of radioisotopes to tumor cells which cause a broad range of damage on DNA, on the replication and repair of DNA, and on the assembly and maintenance of chromosomes. Dosage ranges for radioisotopes vary widely, and depend on the half-life of the isotope, the strength and type of radiation emitted, and the uptake by the neoplastic cells and will be determined by the attending physician.

Radiotherapy may include external or internal radiation therapy. External radiation therapy involves a radiation source outside the subject's body and sending the radiation toward the area of the cancer within the body. Internal radiation therapy uses a radioactive substance sealed in needles, seeds, wires, or catheters that are placed directly into or near the cancer.

### Kits

Pharmaceutical compositions may be assembled into a kit or pharmaceutical system for use in treating GIST. The kits or pharmaceutical systems may include one or more dosage formulations containing a Menin inhibitor and a pharmaceutically acceptable carrier disposed in a suitable container, e.g., tube, vial, ampoule, bottle, syringe, or bag. In some embodiments, the kit or pharmaceutical system may also include one or more dosage formulations of a TKI. In some embodiments, the kit or pharmaceutical system may also include one or more dosage formulations of a MOZ inhibitor. In some embodiments, the kit or pharmaceutical system may also include one or more dosage formulations of a TKI inhibitor and one or more dosage formulations of a MOZ inhibitor. The additional actives may be formulated separately or together, and may be disposed in the same or separate containers The kits or pharmaceutical systems of the disclosure may also comprise printed instructions for using the additional active(s) contained therein.

In some embodiments, the kit includes a Menin inhibitor and a TKI in the same dosage form. In other embodiments, the Menin inhibitor and the TKI are contained in different dosage forms.

These and other aspects of the present disclosure will be further appreciated upon consideration of the following working examples, which are intended to illustrate certain embodiments of the disclosure but are not intended to limit its scope, as defined by the claims.

### EXAMPLES

### Example 1: Materials and Methods

Cell Culture and Virus Production. All cell lines tested negative for mycoplasma infection on routine surveillance (MilliporeSigma Cat #MP0025-1KT). Human embryonic kidney (HEK) 293 FT (Thermo Fisher Scientific Cat #R70007, RRID: CVCL_6911) and the GIST cell lines GIST-T1 (Cosmo Bio Cat #PMC-GIST01-COS, RRID: CVCL_4976; KIT mutation in exon 11 Δ560-578), GIST430 (RRID: CVCL_7040; KIT mutation in exon 11 Δ560-576), GIST48B (RRID: CVCL_M441: KIT-independent), and GIST882 (RRID: CVCL_7044: KIT mutation in exon 13 K642E) were cultured in Dulbecco's modified Eagle's medium containing 10% FBS, 2 mM L-glutamine, 100 mg/ml penicillin, and 100 mg/ml streptomycin. KIT rescue cell lines, which are independent of the KIT enhancer, were generated as previously described (Hemming et al., Cancer Research 79:994-1009 (2019)). Non-commercial cell lines were obtained from the laboratory of Jonathan Fletcher between 2014 and 2016. KIT exons were sequenced to confirm the expected coding mutations and cell identity of GIST cell lines, and cells were thawed from original or derived stocks and used in the described experiments within approximately 3 months. Transfections were performed with X-tremeGene (Roche, Cat #6365809001). Lentiviral production was performed as previously described (Hemming et al., PLOS Biol. 6: e2571-15 (2008)). Briefly, 293 FT cells were cotransfected with pMD2.G (Addgene #12259), psPAX2 (Addgene #12260) and the lentiviral expression plasmid. Viral supernatant was collected at approximately 72 h and debris removed by centrifugation at 1,000 g for 5 min. Cells were transduced with viral supernatant and polybrene at 8 μg/mL by spinoculation at 680 g for 60 min. Drugs were used at the indicated concentrations and included imatinib (LC Laboratories Cat #I-5508), WM-1119 (Selleck Chemicals Cat #S8776), VTP-50469 (gift of Syndax Pharmaceuticals), tazemetostat (Selleck Chemicals Cat #S7128), and EPZ-5676 (Selleck Chemicals Cat #S7062). For growth over time assays, 15×10³ cells were dispensed per well in a 96-well plate, transduced with virus or treated with drug, and cell count performed approximately twice per week on a Guava easy Cyte Flow Cytometer (Luminex Corporation) with normalization of cell count to the control condition.

Genome-scale CRISPR Screen. The Liu Human CRISPR Knockout Library (Addgene #1000000132; Fei et al., Proc. Natl. Acad. Sci. USA 116:25186-95 (2019)) targeting 18,436 genes with 185,634 sgRNAs is divided into two pooled libraries, H1 and H2, containing approximately 5 sgRNAs per gene in each library. Each virion contained an sgRNA, Cas9) and a puromycin resistance gene derived from lentiCRISPRv2. The cell lines GIST-T1 and GIST430 were transduced in duplicate with each library (n=8 total). For each library transduction, 44.64×10⁶ cells were transduced at a target MOI of 0.3, with an estimated library coverage of 134×. Puromycin was applied at 72 h for selection. Cells were passaged at confluency to maintain a library coverage >134× for approximately 30 days. At termination of the experiment, genomic DNA was extracted from 30×10⁶ cells per library. The region of the sgRNA between U6 and EF-1α was amplified from 200 μg of genomic DNA from each experimental replicate in 32 separate 100 μL reactions. The product was pooled and a second PCR reaction was performed to incorporate Illumina adaptors and a 6 bp barcode. A third PCR reaction was performed to enrich for full-length amplicons (primers are detailed in Table 1-Table 3). Final amplicon libraries were purified by agarose gel electrophoresis and extraction with a QIAquick Gel Extraction Kit (Qiagen Cat #28704). Next generation sequencing was performed on a NovaSeq 6000 (Illumina). MAGeCK software (version 0.5.8) was used to analyze screen data (Wang et al., Nat. Protoc. 14 (3): 756-780 (2019)). The “count” command was used to generate read counts of all libraries (n=8) with the initial plasmid library (n=2) used as baseline control. Total counts were normalized between samples to minimize effects of sequencing depth. The maximum likelihood estimate command was used to generate β-scores for each screen, with data normalized to control AAVS1 sgRNAs contained within H1 and H2 libraries. Metascape was used for gene ontology enrichment analysis (Zhou et al., Nat. Commun. 10 (1): 1523 (2019)).

TABLE 3
Nucleic acid sequences of representative primers

| Oligonucleotide | Sequence (5′-->3′) | Application |
|---|---|---|
| sgDOTIL_e1_For | CACCGACGTAGACCGGCAGCGGCCA (SEQ ID | sgRNA |
| | NO: 21) | |
| sgDOTIL_e1_Rev | AAACTGGCCGCTGCCGGTCTACGTC (SEQ ID | sgRNA |
| | NO: 22) | |
| sgDOTIL_e8_For | CACCGCGAATCGCCAACACGAGGTA (SEQ ID | sgRNA |
| | NO: 23) | |
| sgDOTIL_e8_Rev | AAACTACCTCGTGTTGGCGATTCGC (SEQ ID | sgRNA |
| | NO: 24) | |
| GAPDH_For | TGCACCACCAACTGCTTAGC (SEQ ID NO: 25) | qPCR |
| GAPDH_Rev | GGCATGGACTGTGGTCATGAG (SEQ ID NO: | qPCR |
| | 26) | |
| NPR3_For | AGGGTTTGCACACGTCCATC (SEQ ID NO: 27) | qPCR |
| NPR3_Rev | GTCACTGCTCGCACACATGA (SEQ ID NO: 28) | qPCR |
| DUSP6_For | CTCTACGACGAGAGCAGCAG (SEQ ID NO: 29) | qPCR |
| DUSP6_Rev | AGACACCACAGTTCTTGCCC (SEQ ID NO: 30) | qPCR |
| USP1_For | TCGCACCCACACTCATTCG (SEQ ID NO: 31) | qPCR |
| USP1_Rev | GGGCTACCTCTTGAAAGTCCA (SEQ ID NO: 32) | qPCR |
| CRISPR_1st_For | AATGGACTATCATATGCTTACCGTAACTTGA | CRISPR screen 1st |
| | AAGTATTTCG (SEQ ID NO: 33) | round primer |
| CRISPR_1st_R | TCTACTATTCTTTCCCCTGCACTGTGACTGTG | CRISPR screen 1st |
| | GGCGATGTGCGCTCTG (SEQ ID NO: 34) | round primer |
| CRISPR_2nd_ | AATGATACGGCGACCACCGAGATCTACACT | CRISPR |
| For_01 | CTTTCCCTACACGACGCTCTTCCGATCTICAA | screen |
| | GGTCAtcttgtggaaaggacgaaacaccg | 2nd round primer |
| | (SEQ ID NO: 35) | |
| CRISPR_2nd_For_ | AATGATACGGCGACCACCGAGATCTACACT | CRISPR screen |
| 02 | CTTTCCCTACACGACGCTCTTCCGATCTatGC | 2nd round primer |
| | ATAACTtcttgtggaaaggacgaaacaccg | |
| | (SEQ ID NO: 36) | |
| CRISPR_2nd_For_ | AATGATACGGCGACCACCGAGATCTACACT | CRISPR |
| 03 | CTTTCCCTACACGACGCTCTTCCGATCTgatCT | screen |
| | CTGATTtcttgtggaaaggacgaaacaccg | 2nd round primer |
| | (SEQ ID NO: 37) | |
| CRISPR_2nd_For_ | AATGATACGGCGACCACCGAGATCTACACT | CRISPR screen |
| 04 | CTTTCCCTACACGACGCTCTTCCGATCTcgatT | 2nd round primer |
| | ACGTACGtcttgtggaaaggacgaaacaccg | |
| | (SEQ ID NO: 38) | |
| CRISPR_2nd_For_ | AATGATACGGCGACCACCGAGATCTACACT | CRISPR screen |
| 05 | CTTTCCCTACACGACGCTCTTCCGATCTtcgatT | 2nd round primer |
| | ACGCGTTtcttgtggaaaggacgaaacaccg | |
| | (SEQ ID NO: 39) | |
| CRISPR_2nd_For_ | AATGATACGGCGACCACCGAGATCTACACT | CRISPR screen |
| 06 | CTTTCCCTACACGACGCTCTTCCGATCTatcgat | 2nd round primer |
| | CTCAGTGAtcttgtggaaaggacgaaacaccg | |
| | (SEQ ID NO: 40) | |
| CRISPR_2nd_For_ | AATGATACGGCGACCACCGAGATCTACACT | CRISPR screen |
| 07 | CTTTCCCTACACGACGCTCTTCCGATCTgatcga | 2nd round primer |
| | tTCTGATATtcttgtggaaaggacgaaacaccg | |
| | (SEQ ID NO: 41) | |
| CRISPR_2nd_For_ | AATGATACGGCGACCACCGAGATCTACACT | CRISPR screen |
| 08 | CTTTCCCTACACGACGCTCTTCCGATCTcgatcg | 2nd round primer |
| | atCATATGCTtcttgtggaaaggacgaaacaccg | |
| | (SEQ ID NO: 42) | |
| CRISPR_2nd_For_ | AATGATACGGCGACCACCGAGATCTACACT | CRISPR screen |
| 09 | CTTTCCCTACACGACGCTCTTCCGATCTacgatc | 2nd round primer |
| | gatCGTAATTAtcttgtggaaaggacgaaacaccg | |
| | (SEQ ID NO: 43) | |
| CRISPR_2nd_For_ | AATGATACGGCGACCACCGAGATCTACACT | CRISPR screen |
| 10 | CTTTCCCTACACGACGCTCTTCCGATCTtACG | 2nd round primer |
| | TACTCtcttgtggaaaggacgaaacaccg | |
| | (SEQ ID NO: 44) | |
| CRISPR_2nd_Rev_ | CAAGCAGAAGACGGCATACGAGATCAAGGT | CRISPR screen |
| 01 | CAGTGACTGGAGTTCAGACGTGTGCTCTTCC | 2nd round primer |
| | GATCTacgatcgatTCTACTATTCTTTCCCCTGCA | |
| | CTGT (SEQ ID NO: 45) | |
| CRISPR_2nd_Rev_ | CAAGCAGAAGACGGCATACGAGATGCATAA | CRISPR screen |
| 02 | CTGTGACTGGAGTTCAGACGTGTGCTCTTCC | 2nd round primer |
| | GATCTcgatcgatTCTACTATTCTTTCCCCT | |
| | GCACTGT (SEQ ID NO: 46) | |
| CRISPR_2nd_Rev_ | CAAGCAGAAGACGGCATACGAGATCTCTGA | CRISPR screen |
| 03 | TTGTGACTGGAGTTCAGACGTGTGCTCTTCC | 2nd round primer |
| | GATCTgatcgatTCTACTATTCTTTCCCCTG | |
| | CACTGT (SEQ ID NO: 47) | |
| CRISPR_2nd_Rev_ | CAAGCAGAAGACGGCATACGAGATTACGTA | CRISPR screen |
| 04 | CGGTGACTGGAGTTCAGACGTGTGCTCTTCC | 2nd round primer |
| | GATCTatcgatTCTACTATTCTTTCCCCTGC | |
| | ACTGT (SEQ ID NO: 48) | |
| CRISPR_2nd_ | CAAGCAGAAGACGGCATACGAGATTACGCG | CRISPR screen |
| Rev_05 | TTGTGACTGGAGTTCAGACGTGTGCTCTTCC | 2nd round primer |
| | GATCTtcgatTCTACTATTCTTTCCCCTGCA | |
| | CTGT (SEQ ID NO: 49) | |
| CRISPR_2nd_ | CAAGCAGAAGACGGCATACGAGATCTCAGT | CRISPR screen |
| Rev_06 | GAGTGACTGGAGTTCAGACGTGTGCTCTTCC | 2nd round primer |
| | GATCTcgatTCTACTATTCTTTCCCCTGCAC | |
| | TGT (SEQ ID NO: 50) | |
| CRISPR_2nd_ | CAAGCAGAAGACGGCATACGAGATTCTGAT | CRISPR |
| Rev_07 | ATGTGACTGGAGTTCAGACGTGTGCTCTTCC | screen |
| | GATCTgatTCTACTATTCTTTCCCCTGCACTGT | 2nd round primer |
| | (SEQ ID NO: 51) | |
| CRISPR_2nd_ | CAAGCAGAAGACGGCATACGAGATCATATG | CRISPR |
| Rev_08 | CTGTGACTGGAGTTCAGACGTGTGCTCTTCC | screen |
| | GATCTatTCTACTATTCTTTCCCCTGCACTGT | 2nd round primer |
| | (SEQ ID NO: 52) | |
| CRISPR_2nd_ | CAAGCAGAAGACGGCATACGAGATCGTAAT | CRISPR screen |
| Rev_09 | TAGTGACTGGAGTTCAGACGTGTGCTCTTCC | 2nd round primer |
| | GATCTUTCTACTATTCTTTCCCCTGCACTGT | |
| | (SEQ ID NO: 53) | |
| CRISPR_2nd_ | CAAGCAGAAGACGGCATACGAGATACGTAC | CRISPR screen |
| Rev_10 | TCGTGACTGGAGTTCAGACGTGTGCTCTTCC | 2nd round primer |
| | GATCTacgatcgatTCTACTATTCTTTCCCC | |
| | TGCACTGT (SEQ ID NO: 54) | |
| CRISPR_3rd_ | AATGATACGGCGACCACCGAGATC (SEQ ID | CRISPR screen |
| For | NO: 55) | 3rd round primer |
| CRISPR_3rd_ | CAAGCAGAAGACGGCATACGAGAT (SEQ ID | CRISPR screen |
| R | NO: 56) | 3rd round primer |

Cloning and CRISPR. Cell lines stably expressing a human codon-optimized Streptococcus pyogenes Cas9 (Addgene #73310) were generated by viral transduction. CRISPR single-guide RNAs (sgRNAs) were designed using CHOPCHOP (Labun et al., Nucleic Acids Research 44:W272-6 (2016)) (chopchop.cbu.uib.no), cloned into Lenti-sgRNA-EFS-GFP (LRG, Addgene #65656) modified with GFP replaced by copGFP linked to a puromycin resistance gene by a 2A peptide, and detailed in Table 1-Table 3. The BioID expression vectors were synthesized with codon optimization to alter sgRNA binding sequences (Twist Bioscience). Dependency Map (DepMap) portal data was accessed through depmap.org (Barretina et al., Nature 483:603-7 (2012)), utilizing the CRISPR (Avana) Public 20Q3 through 20Q4 releases.

Cell cycle and apoptosis. Cell cycle analysis was performed following drug treatment for 72 h (imatinib) or 8 days (VTP-50469, WM-1119). Cells were trypsinized, washed in PBS and fixed in 70% ethanol. Propidium iodide at 25 μg/mL (Life Technologies Cat #P1304MP) and RNAse A at 0.2 mg/mL (Thermo Fischer Scientific Cat #EN0531) were used to stain nuclear DNA. Analysis was performed on a Guava easyCyte Flow Cytometer (Luminex Corporation), and single cells were assessed for nuclear content using Guava InCyte software. Apoptosis and cell death were measured following 72 h of drug treatment using Guava Nexin Reagent (Luminex Corporation Cat #4500-0450) per manufacturer's recommendations. Non-apoptotic cells stain negative for Annexin V and 7-AAD, early apoptotic cells stain positive for Annexin V but negative for 7-AAD and late apoptotic and dead cells stain positive for both Annexin V and 7-AAD. Staining was assayed on a Guava easyCyte Flow Cytometer and data analyzed using Guava InCyte software.

Quantitative RT-PCR. Cells were trypsinized and washed in PBS for RNA extraction using the RNeasy Mini Kit (Qiagen Cat #74106). Libraries of cDNA were made using SuperScript IV VILO cDNA Synthesis Kit (Invitrogen Cat #11766050). RT-PCR was performed using Power SYBR Green PCR Master Mix (Life Technologies Cat #4367659) on a QuantStudio 6 Flex Real-Time PCR System (Thermo Fischer Scientific). Relative mRNA levels were calculated by the AACt method using GAPDH expression as reference. Primers are listed in Table 1-Table 3.

RNA-seq. Total RNA was isolated using a RNeasy Plus Kit (Qiagen Cat #74136), and concentration measured by Nanodrop (Thermo Fisher Scientific) and quality by TapeStation 4200 (Agilent). Library preparation was performed using the NEBNext Ultra II DNA Library Prep Kit (New England Biolabs Cat #E7645S). Paired-end 150 bp sequencing was performed on a NovaSeq 6000 (Illumina). RNA-seq data were aligned to hg19 using STAR (Dobin et al., Bioinformatics 29:15-21 (2012)) with expression quantification using Cufflinks (Trapnell et al., Nat. Biotechnol. 28:511-5 (2010)) to generate gene expression values in fragments per kilobase of transcript per million mapped reads (FPKM) units. Gene set enrichment analysis (GSEA, RRID: SCR_003199) (Subramanian et al., Proc. Natl. Acad. Sci. USA 102:15545-50 (2005)) was performed using Hallmark gene lists in the Molecular Signatures Database.

ChIP-seq and Cut&Tag. For ChIP-seq, approximately 20×10⁶ cells were incubated in 1% formaldehyde for 10 min. Following fixation, excess formaldehyde was quenched with glycine at 0.125 M for 5 min. Samples were washed with PBS, and intact nuclei suspended in SDS Buffer (0.5% SDS, 50 mM Tris, 100 mM NaCl, 5 mM EDTA with protease inhibitor cocktail (Roche Cat #11873580001)) and sonicated in a E220 Focused-ultrasonicator (Covaris, Inc.). Sonicated samples were spun 20,000 g for clarification and supernatant diluted to <0.1% SDS then incubated with Dynabeads Protein A (Life Technologies Cat #10002D) pre-bound with antibody (H3K9ac, Active Motif Cat #39137, RRID:AB_2561017; H3K4me3, Abcam Cat #ab8580, RRID:AB_306649; BRPF1, Thermo Fisher Scientific Cat #PA5-27783, RRID:AB_2545259; KAT6A, Cell Signaling Technology Cat #78462: HA, Cell Signaling Technology Cat #3724, RRID:AB_1549585: DOT1L, Cell Signaling Technology Cat #77087, RRID:AB_2799889; H3K79me2. Cell Signaling Technology Cat #5427. RRID:AB_10693787) overnight. Samples were washed serially with Buffer A (150 mM NaCl. 5 mM EDTA. 5% sucrose. 1% Triton X-100, 0.2% SDS. 20 mM Tris), Buffer B (5 mM EDTA. 1% Triton X-100, 0.1% Deoxycholate. 20 mM Tris). Buffer C (250 mM LiCl. 1 mM EDTA. 0.5% NP40, 0.5% Deoxycholate. 10 mM Tris) and TE following resuspension of beads in Elution Buffer (200 mM NaCl. 100 mM NaHCO₃, 1% SDS) and incubation at 65° C. to reverse crosslinks for 12-15 h. DNA was purified using AMPureXP beads (Beckman Coulter Cat #A63881) per manufacturer recommendation, and quality assessed by Qubit dsDNA HS Assay Kit (Life Technologies Cat #Q32854) and TapeStation 4200 (Agilent). Sequencing libraries were prepared using a ThruPLEX DNA-seq Kit (Takara Bio Cat #R400675) and sequenced on a NextSeq 500 or 550 System (Illumina). ChIP-seq spike-in normalization was performed by pre-binding spike-in antibody (Active Motif Cat #61686) together with the IP antibody of interest to Dynabeads. Equal amounts of Drosophila melanogaster chromatin (Active Motif Cat #53083) was added to prepared GIST cell chromatin per manufacturers recommendations. Resultant sequenced samples were aligned to the Drosophila genome, with total Drosophila read counts used to normalize Homo sapiens read counts across samples.

Cut&TAG was performed as previously described (Kaya-Okur et al., Nat. Commun. 10 (1): 1930 (2019)) using a protein A and Tn5 Transposase fusion protein (Addgene #124601). In brief. 100,000 GIST-T1 cells were washed in Wash Buffer (20 mM HEPES pH 7.5, 150 mM NaCl. 0.5 mM Spermidine. protease inhibitor cocktail) and bound to Concanavalin A beads (Bangs Laboratories Cat #BP531) for 15 min at room temperature. Bound cells were resuspended in 50 μL Dig Wash Buffer (20 mM HEPES pH 7.5, 150 mM NaCl. 0.5 mM Spermidine. protease inhibitor cocktail. 2 mM EDTA. 0.05% Digitonin) and incubated with antibody diluted 1:100 overnight at 4° C. (Menin. Bethyl Cat #A300-105A. RRID:AB_2143306; MLLIn. Bethyl Cat #A300-086A. RRID:AB_242510). A magnet was used to collect beads, and cells were resuspended in 100 μL Dig-Wash buffer with a secondary antibody diluted 1:100 and incubated at room temperature for 30 min. Cells were washed three times in Dig-Wash buffer and resuspended in Dig-Med Buffer (0.05% Digitonin. 20 mM HEPES. pH 7.5, 300 mM NaCl. 0.5 mM Spermidine. protease inhibitor cocktail) containing 1:250 pA-Tn5 transposase and incubated at room temperature for 1 h. Cells were washed three times in Dig-Med Buffer and resuspended in 300 μL Tagmentation Buffer (10 mM MgCl2 in Dig-Med Buffer) and incubated at 37° C. for 1 h. Tagmentation was stopped by adding 10 μL of 0.5 M EDTA. 3 μL of 10% SDS and 2.5 μL of 20 mg/mL Proteinase K (Invitrogen Cat #25530049) and samples incubated 50° C. for 1 h. Tagmented DNA was purified by phenol:cholorphorm:isoamyl alcohol extraction, and aqueous layer subjected to ethanol precipitation, and DNA was resuspended in 30 μL TE. For each sample, 21 μL DNA was mixed with a universal i5 and uniquely barcoded i7 primer and amplified using NEBNext High Fidelity 2×PCR Master Mix (New England Biolabs Cat #M0541S) in a thermocycler using the following conditions: 98° C. for 30 sec: 14 cycles of 98° C. for 10 sec, 63° C. for 10 sec: 72° C. for 2 min. DNA was purified with AMPureXP beads per manufacturer recommendation, and quality assessed by Qubit dsDNA HS Assay Kit and TapeStation 4200. Samples were sequenced on a NextSeq 550 System (Illumina).

All sequencing data were aligned to the human reference genome assembly hg19 using Bowtie2 (Langmead et al., Genome Biol. 10: R25.1—R25.10 (2009)). Normalized read density was calculated using Bamliquidator (version 1.0) read density calculator. Aligned reads were extended by 200 bp and the density of reads per base pair was calculated. In each region. the density of reads was normalized to the total number of million mapped reads, generating read density in units of reads per million mapped reads per bp (rpm/bp). Peak finding was performed by Model-based Analysis for ChIP-seq (MACS, version 1.4.2, Feng et al., Nature Protocols 7:1728-40 (2012)), and ROSE2 (Lovén et al., Cell 153:320-34 (2013)) was used to identify regions of signal enrichment. Individual ChIP-seq track displays were generated using bamplot (github.com/linlabbcm). Heat map visualizations of ChIP-seq data were generated using ChAsE (Younesy et al., Bioinformatics 32:3324-6 (2016)).

Immunobloting. Cells were lysed in RIPA buffer containing protease inhibitor cocktail (Roche Cat #11873580001) and centrifuged at 14,000 g for 10 min to remove genomic DNA and debris. Protein concentrations were determined using a bicinchoninic acid-based assay (Pierce Biotechnology Cat #23225). Protein samples were subjected to SDS-PAGE and Western blotting with the following antibodies: HA (1:1,000, Cell Signaling Technology Cat #2367, RRID:AB_10691311), MEAF6 (1:500, Proteintech Cat #26465-1-AP. RRID:AB_2880524), Actin (1:1,000, Cell Signaling Technology Cat #4967, RRID:AB_330288), Menin (1:10,000, Bethyl Cat #A300-105A, RRID:AB_2143306), or streptavidin-HRP (1:40,000, Abcam Cat #ab7403). Western blots were probed with anti-mouse or anti-rabbit secondary antibodies and detected using the Odyssey CLx infrared imaging system (LI-COR Biosciences), or streptavidin-HRP by chemiluminescence (MilliporeSigma Cat #WBKLS0500). Immunoblots shown are representative of at least three independent experiments.

Mass Spectrometry and BioID. GIST-T1 cell lines were generated which stably expressed control or experimental mutant biotin ligase (BirA* R118G)-tagged fusion proteins. 24 h biotin-labeled whole cell lysate was subject to affinity pulldown overnight at 4° C. using streptavidin-sepharose beads (GE Healthcare Cat #17-5113-01). Beads were washed three times in 2% SDS in 50 mM Tris. twice in BioID buffer (50 mM Tris. 500 mM NaCl. 0.4% SDS), six times in 50 mM Tris and resuspended in 100 μL of ammonium bicarbonate. Samples were subject to tryptic digestion, and beads and salts removed in a reverse-phase cleanup step. Extracts were dried on a speed-vac, and later reconstituted in 5-10 ul of 2.5% acetonitrile and 0.1% formic acid. A nano-scale reverse-phase HPLC capillary column was created by packing 2.6 μm C18 spherical silica beads into a fused silica capillary (100 μm inner diameter x˜30 cm length) with a flame-drawn tip. After equilibrating the column each sample was loaded via a Famos Auto Sampler (LC Packings). A gradient was formed and peptides were eluted with increasing concentrations of 97.5% acetonitrile and 0.1% formic acid. As peptides eluted they were subjected to electrospray ionization and then entered into an LTQ Orbitrap Velos Pro ion-trap mass spectrometer (Thermo Fisher Scientific). Peptides were detected. isolated, and fragmented to produce a tandem mass spectrum of specific fragment ions for each peptide. Peptide sequences (and hence protein identity) were determined by matching protein databases with the acquired fragmentation pattern by Sequest (Thermo Fisher Scientific). All databases include a reversed version of all the sequences and the data was filtered to between a one and two percent peptide false discovery rate. Label-free quantification of signal intensity was used in replicate samples for quantitative comparisons. Heat maps of log₂ fold change in signal compared to DMSO were generated using Morpheus (software. broadinstitute.org/morpheus/).

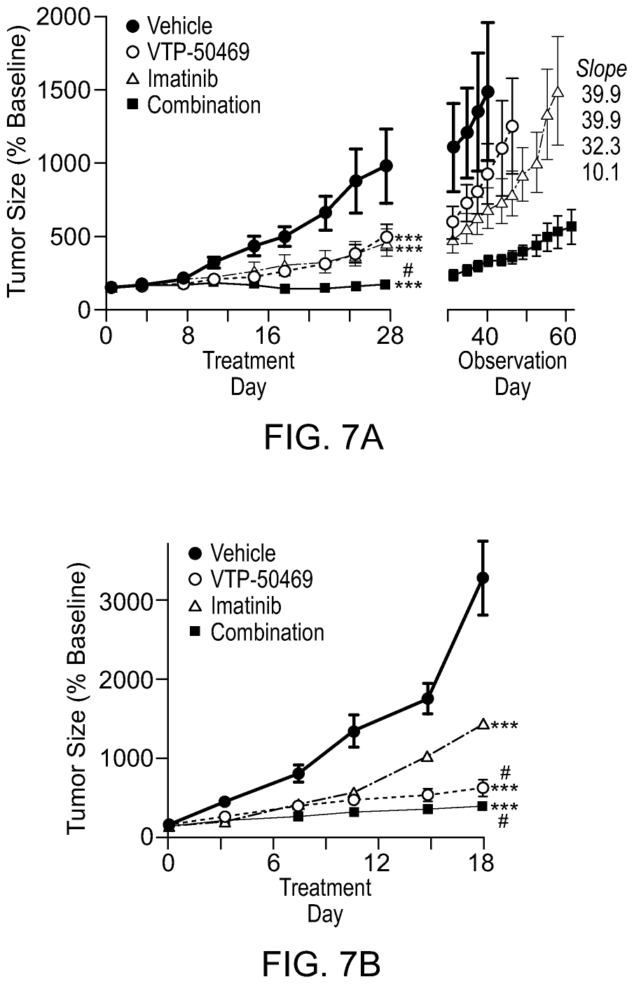

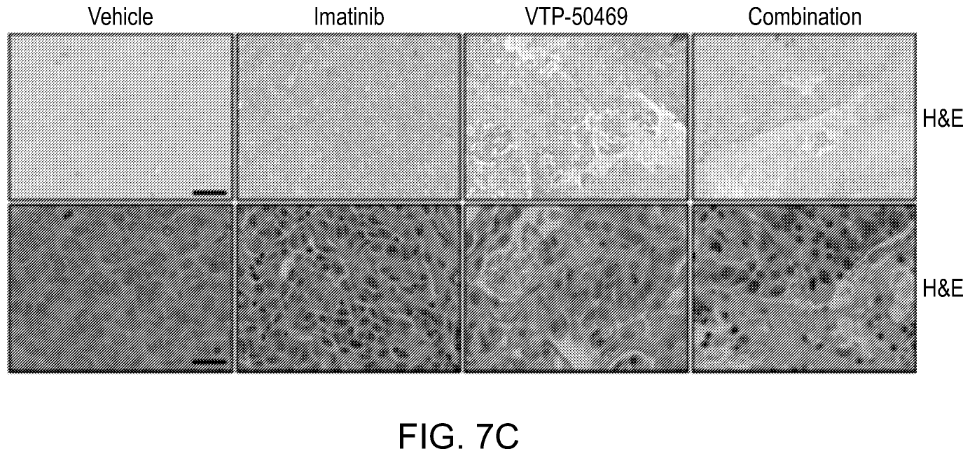

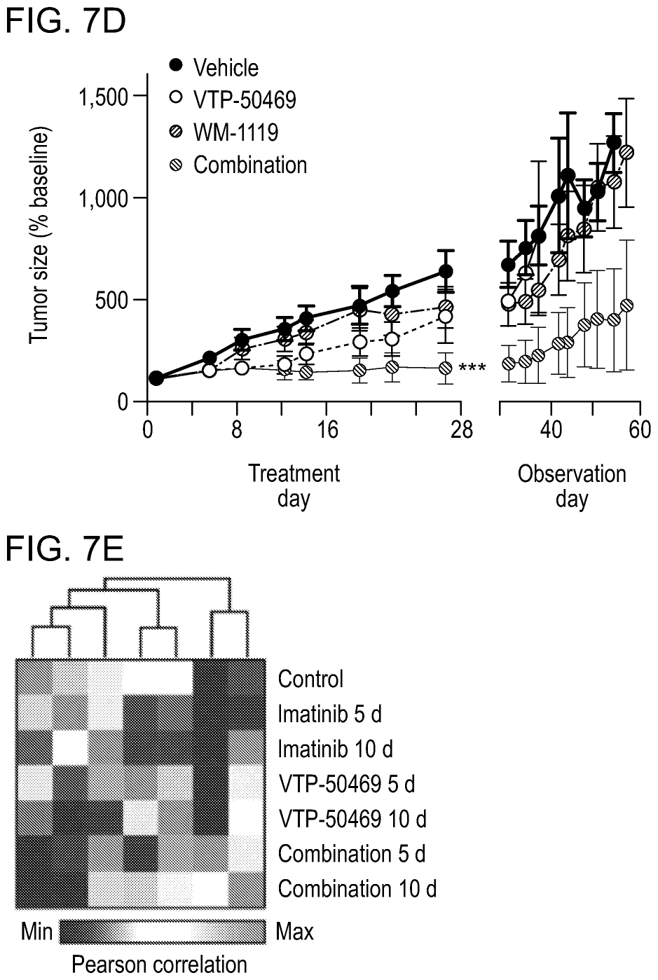

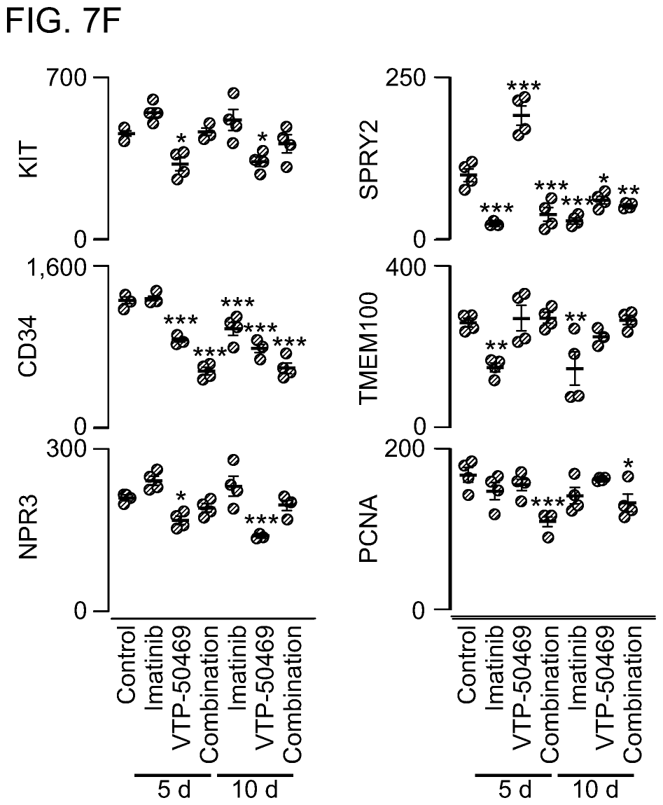

Xenograft Models. The PG27 patient derived xenograft was obtained from a patient undergoing clinically indicated surgery and following written informed consent to a Dana-Farber Cancer Institute IRB-approved research protocol. Cryopreserved tumor or the GIST-T1 cell line mixed 1:1 with matrigel were implanted subcutaneously into 6-week-old female nude mice (NU/NU; Charles River Laboratories). GIST-T1 tested negative for mycoplasma and rodent pathogens (Charles River Laboratories). For in vivo assessment of CRISPR/Cas9)-modified cell line growth. GIST-T1/Cas9 cells were treated with the indicated sgRNAs and selected with puromycin in vitro for 14 days prior to bilateral flank implantation. For drug treatment studies, singly engrafted mice were enrolled into treatment groups when tumors reached approximately 100-200 mm³ in size, as measured by calipers and determined by the tumor volume equation: volume=long diameter²×short diameter×0.5. Mice were randomly assigned to treatment groups administered imatinib (50 mg/kg gavage daily. 5 days per week). WM-1119 (50 mg/kg gavage 3 times daily. 7 days per week). VTP-50469 (0.1% in chow) or combination treatments. Imatinib was administered below the maximal tolerated dose to facilitate testing of combination therapy. No statistical methods were used to predetermine sample size, and no animals died during drug treatment. Two GIST-T1 cell line xenograft mice in the control groups were excluded from analysis as the initially measured subcutaneous implant failed to grow. One outlier tumor-bearing mouse in the VTP-50469 arm in FIG. 7D was excluded due to early termination from rapid tumor growth. Tumors were dissected and fixed in 10% formalin for corollary studies including H&E staining and immunohistochemistry of sectioned tumors. 4 μm sections were cut from fixed and embedded tumors and stained with Ki-67 (1:400, Cell Signaling Technology Cat #9027) and cleaved caspase-3 (1:250, Cell Signaling Technology Cat #9579). Reactions were developed using DAB (Cell Signaling Technology Cat #8059) or NovaRed (Vector Laboratories Cat #SK-4800) substrate kits per manufacturer recommendations. All procedures were conducted under protocols approved by the Institutional Animal Care and Use Committee at Dana-Farber Cancer Institute.

Statistical analysis. Center values, error bars, P-value cutoffs, number of replicates and statistical tests are identified in the corresponding figure legends. For box plots, the box extends from the 25th to 75th percentiles, with the center line indicating the median and whiskers drawn to the 10th and 90th percentile. Samples sizes were not predetermined.

Data and Materials Availability. Novel sequencing data is available through the GEO Publication Reference ID GSE172154. Additional RNA-seq, ATAC-seq and ChIP-seq data sets analyzed in this study include GSE95864 (Hemming et al., Proc. Natl. Acad. Sci. U.S.A 115 (25):E5746-E5755 (2018)), GSE113207 and GSE113217 (Hemming et al., Cancer Res. 79 (5): 994-1009 (2019)).

### Example 2: Genome-Wide Screening Identifies GIST Epigenetic Dependences

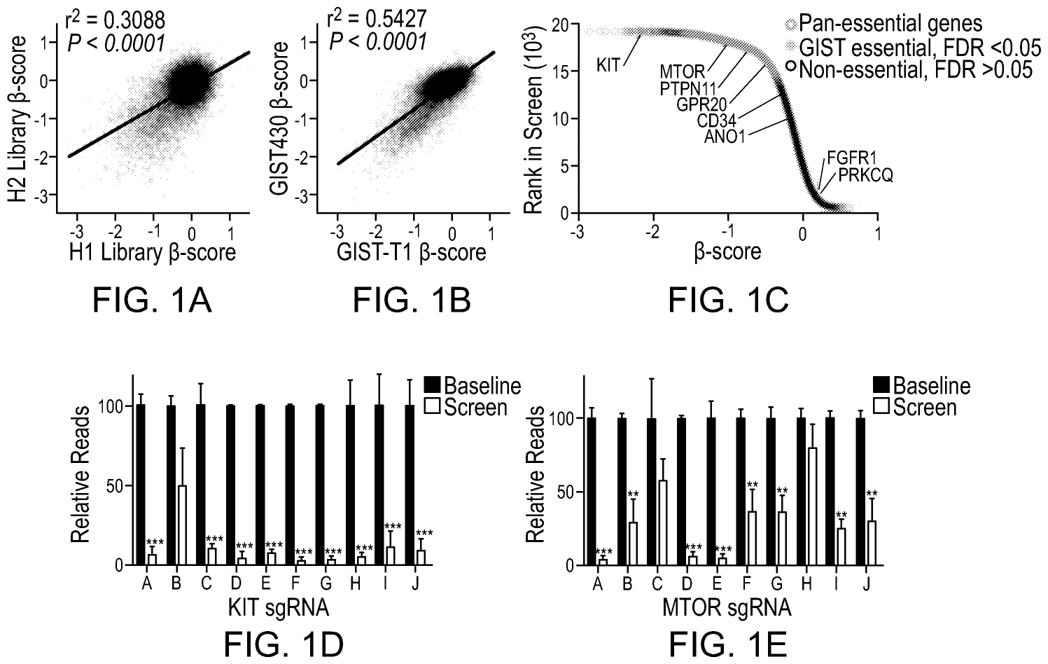

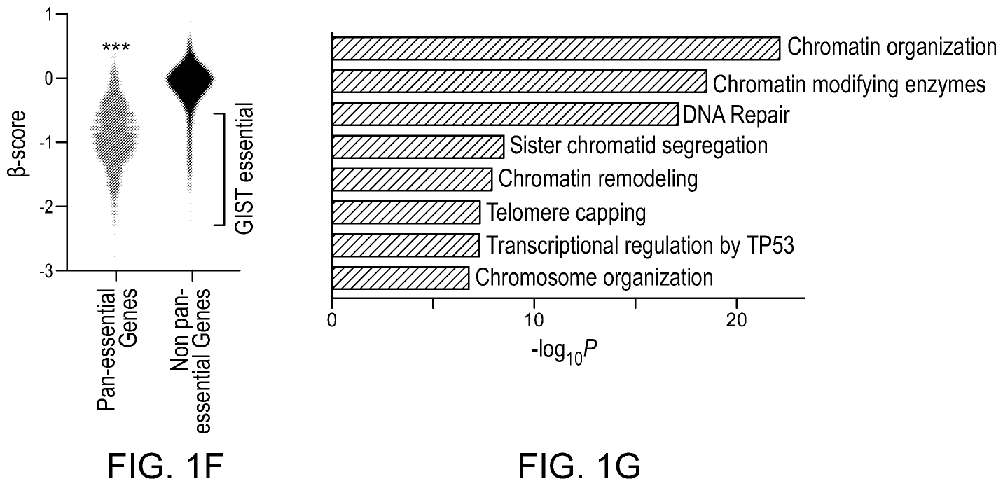

The global transcriptional and enhancer landscape of GIST was characterized using RNA-seq, chromatin immunoprecipitation with sequencing (ChIP-seq), and assay for transposase-accessible chromatin using sequencing (ATAC-seq). These investigations focused on TFs relevant to GIST biology, including core TFs such as ETV1, FOXF1, HIC1, and OSR1 present across GIST samples, and accessory TFs BARXI and HAND1 expressed in disease-state specific patterns (Hemming et al., Cancer Res. 79 (5): 994-1009 (2019)). However, how these TFs integrate with other epigenetic regulators to establish the GIST-associated gene expression program was unknown. A genome-wide CRISPR/Cas9-based dropout screen in two KIT mutant GIST cell lines, GIST-T1 and GIST430, was performed to establish which genes are essential in GIST biology. A split-library approach utilizing paired human whole-genome sgRNA libraries (denoted H1 and H2) was used, with approximately 5 sgRNAs per gene in each library targeting 18.436 genes with a total of 185,634 sgRNAs in the screen. Significant correlation in dependency (B) scores was observed between the H1 and H2 libraries as illustrated in FIG. 1A, as well as between the two GIST cell lines, as illustrated in FIG. 1B (per cell line, n=4 libraries). Data sets were then merged for subsequent analysis to improve statistical power (n=4 per library). Pearson correlation was performed in FIGS. 1A and 1B with P value and r2 as shown. Genes were stratified as ‘pan-essential’, having been previously determined to be universally essential for cellular viability (Blomen et al., Science 350:1092-6 (2015), Wang et al., Science 350:1096-101 (2015)), ‘GIST essential’ with an FDR<0.05 in the screen but absent from the pan-essential list, or ‘Non-essential’, see FIG. 1C, where the pan-essential genes (Blomen et al., Science 350:1092-6 (2015)) are indicated in blue, genes significantly depleted in GIST but not pan-essential are in red, and non-essential genes lacking significant depletion are in gray. Select GIST-associated genes are labeled.

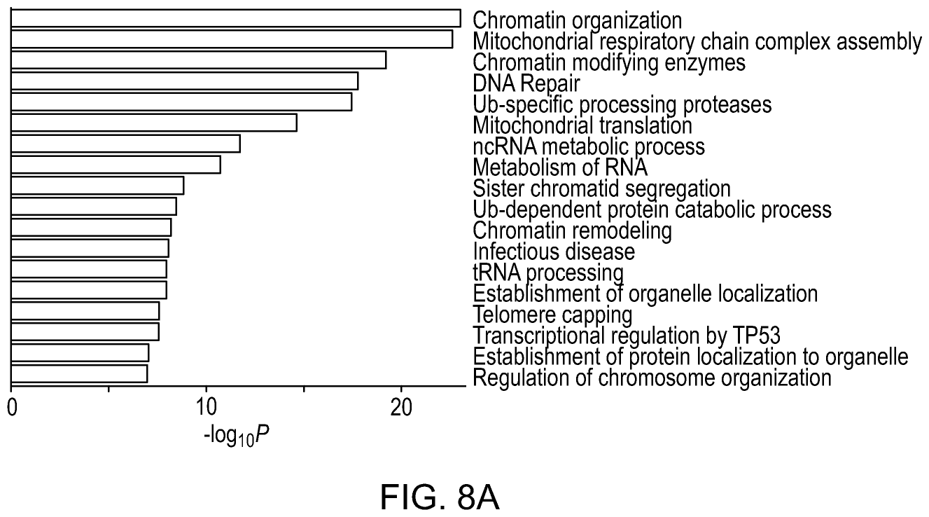

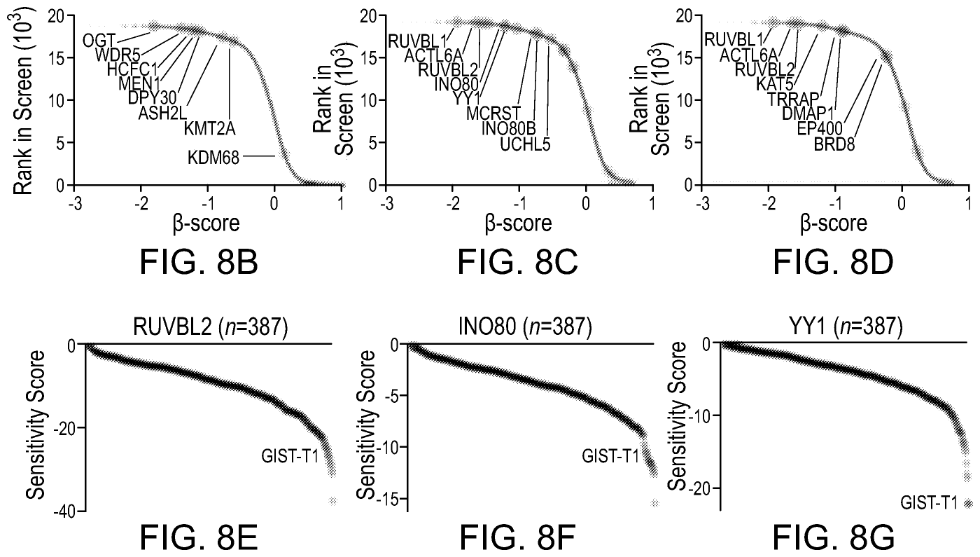

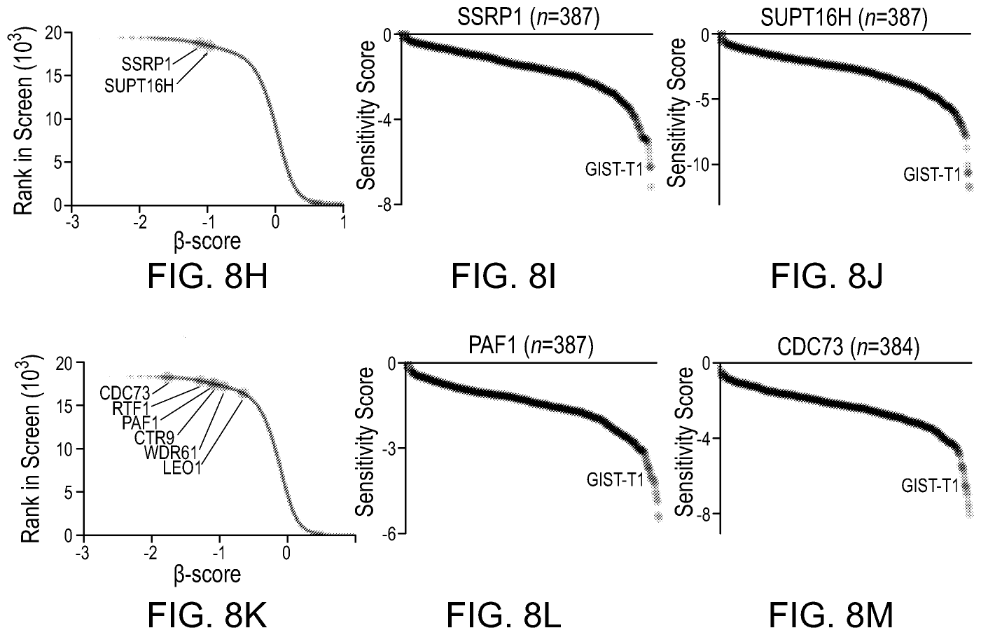

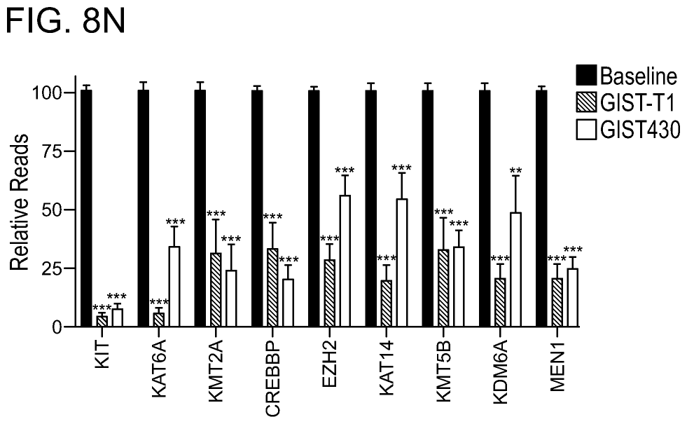

As anticipated, KIT was among the strongest detected dependencies, with sgRNA-level data showing near complete dropout of most (9/10) sgRNAs during the screen, as illustrated in FIG. 1D (n=2 per library). Other canonical downstream signaling mediators of the KIT pathway, such as mTOR, showed significant dropout in the screen, but less so compared to KIT, see FIG. 1E (n=4). The ‘GIST essential’ subset of genes out of the pan-essential (n=1,702) and non-pan-essential (n=16,757), were focused on to identify biological processes which may be specifically enriched in GIST, as illustrated in FIG. 1F. Conditions in FIGS. 1D, 1E and IF were compared by t-test (compared to non-essential genes or baseline sgRNA: **, P<0.01: ***, P<0.001). These unique dependencies in GIST were evaluated by gene ontology enrichment analysis, which revealed that 8 of the top 18 terms were associated with epigenetic regulatory mechanisms including chromatin and chromosomal organization, see FIGS. 1G and 8A. Taken together, data from these unbiased dependency screens characterize GIST as having remarkable reliance upon epigenetic mechanisms to maintain its oncogenic program (Tabone et al., Biochim. Biophys. Acta. 1741 (1-2): 165-72 (2005)).

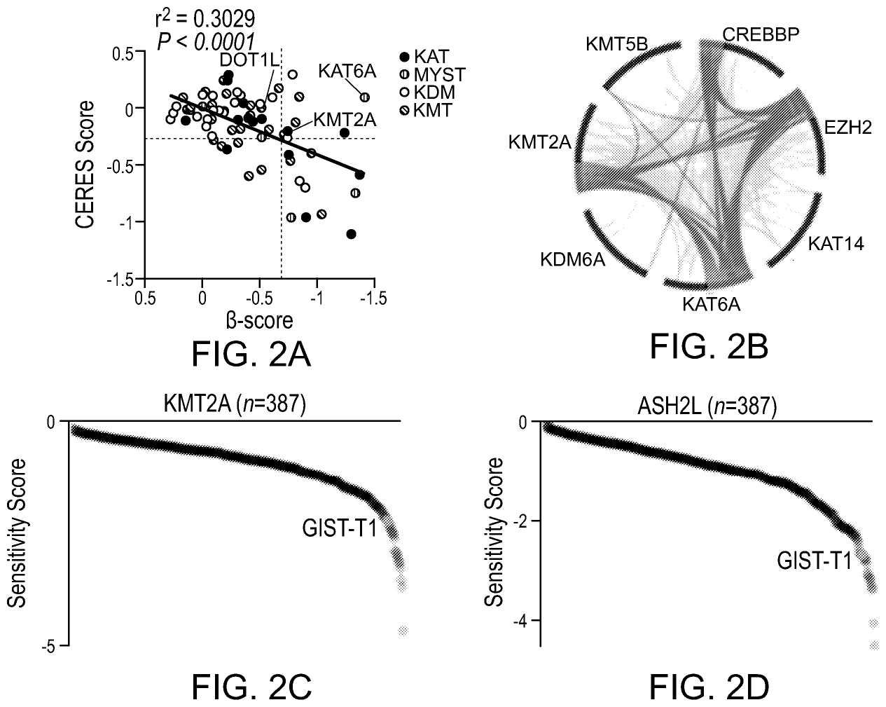

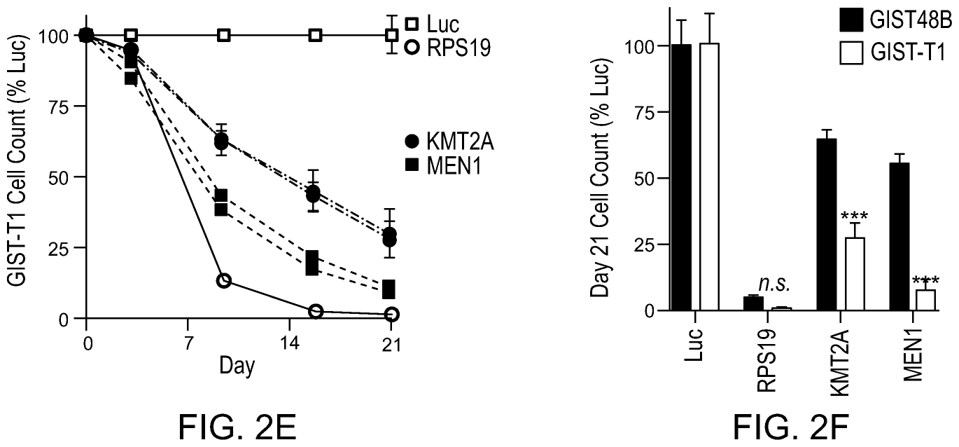

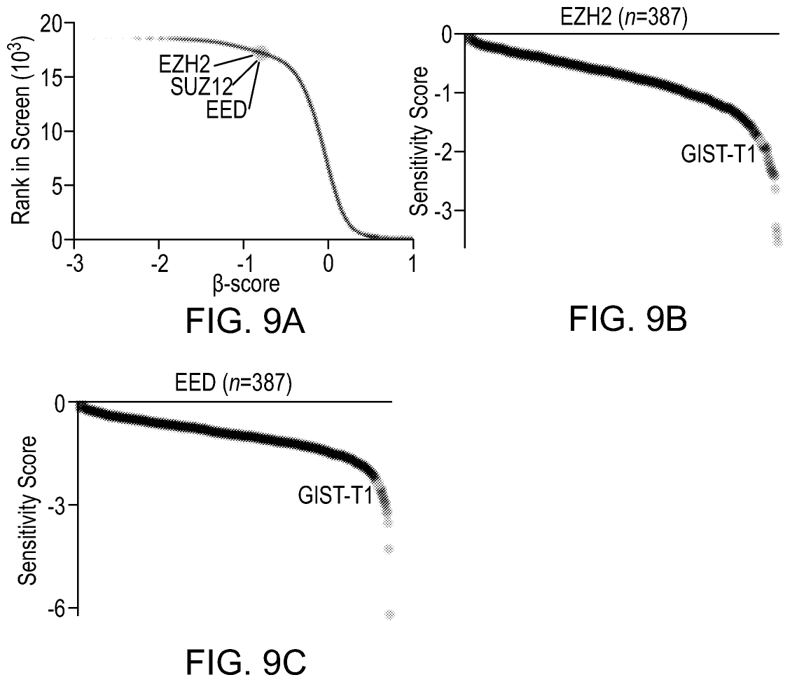

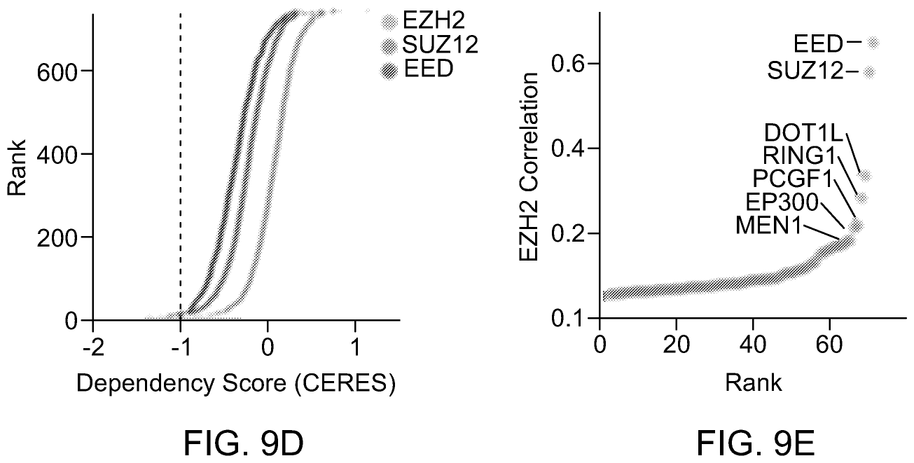

To better define which chromatin regulatory complexes may be most relevant and unique to GIST biology.-scores were compared for all chromatin modifying enzymes in GIST cell lines to analogous CERES dependency scores averaged across all cell lines in the DepMap project (Barretina et al., Nature 483:603-7 (2012)). Only 7 of the 77 assessed chromatin modifying enzymes were unique and essential to GIST, with β-score<−0.7 and CERES score>−0.25 (FIG. 2A and FIG. 8N), with dependency score cutoffs chosen to select for chromatin regulators likely to be unique dependencies and labeling for the Person correlation coefficient. Enriched enzymes included members of the lysine acetyltransferase (KAT), MYST, lysine demethylase (KDM), and lysine methyltransferase (KMT) families. The dotted lines divide the plot into quadrants, with the upper quadrant containing 7 genes that were dependencies in GIST but not common dependencies across DepMap cell lines. To establish which modifying enzymes may function collaboratively to maintain the epigenome. the gene-level co-dependency data was analyzed within DepMap. Comparative analysis of the top 50 co-dependencies of each chromatin modifying enzyme showed the highest interaction at gene and ontology term levels between KMT2A. EZH2, and KAT6A, as illustrated in FIG. 2B. suggesting their genetic co-dependency. In FIG. 2B. red lines connect genes shared on multiple co-dependency lists. Blue lines connect genes that fall into the same ontology term. KMT2A. the catalytic member of the Menin-MLL complex, also had multiple recognized complex members with significant dependency scores including MEN1/Menin and ASH2L, as illustrated in FIG. 8B. As no DepMap CRISPR dependency screening efforts have profiled GIST, available comparative screening results from Project DRIVE (McDonald et al., Cell 170:577-592 (2017)), which included GIST-T1 among nearly 400 cell lines profiled by RNAi. were leveraged. Among all cell lines profiled. KMT2A and ASH2L were in the top 5% highest sensitivity with GIST-T1, as illustrated in FIGS. 2C-2F. further indicating through an independent and comparative screening approach the essential and co-dependent nature of the MOZ and Menin-MLL complexes in GIST. Several other chromatin regulatory complexes were found to have multiple members with significant dependencies in the screen and also showed enrichment for GIST-T1 in Project Drive, including members of the INO80 complex. NuA4 histone acetyltransferase complex. FACT complex, and PAF1 complex, as illustrated in FIGS. 8C-8M. FIGS. 81-8J show Ranked Sensitivity Score from Project Drive cell lines (n=387) for members of the FACT complex, with GIST-T1 highlighted in red. FIGS. 8L-8M show Ranked Sensitivity Score from Project Drive cell lines (n=387) for select members of the PAF1 complex, with GIST-T1 highlighted in red. EZH2. SUZ12 and EED. the core members of the PRC2 complex (Laugesen et al., Cold Spring Harb. Perspect. Med. 6 (9): a026575 (2016)), were all dependencies in the screen, and GIST-T1 had among the highest sensitivity scores for EZH2 and EED in Project DRIVE, as illustrated in FIGS. 9A-9C. FIGS. 9B-9C show Ranked Sensitivity Score from Project Drive cell lines (n=387) for select members of the PRC2 complex, with GIST-T1 highlighted in red. Though few cell lines in DepMap had significant dependencies on core PRC2 complex members. the top correlated co-dependencies of EZH2. including DOT1L. EP300 and MEN1. showed overlap with MOZ and Menin-MLL complex co-dependencies, as illustrated in FIG. 9E. indicating the complementary function of the transcriptionally repressive PRC2 complex.

To validate dependency upon Menin-MLL complex members, a growth-over time assay utilizing unique sgRNAs targeting Menin-MLL complex members KMT2A and MEN1 was performed. For each gene, and with two independent sgRNAs per gene, sgRNA treatment significantly reduced cell proliferation as illustrated in FIG. 2E, sgRNAs targeting Luc and RPS19 are shown in open boxes and circles, respectively (n=3 per sgRNA). GIST48B was used to compare the relative toxicity of these sgRNAs to a control cell line, GIST48B has a similar growth rate as GIST-T1 but through in vitro selection has lost KIT expression and the GIST-associated epigenetic and transcriptional program (Hemming et al., Proc. Natl. Acad. Sci. U.S.A. 115 (25):E5746-E5755 (2018), Hemming et al., Cancer Res. 79 (5): 994-1009 (2019)). While all sgRNAs targeting Menin-MLL complexes significantly reduce GIST-T1 cell proliferation, GIST48B showed little or no change in cell proliferation, as illustrated in FIG. 2F, where n=6 per gene from two sgRNAs. Data were analyzed by two-way ANOVA with Tukey's multiple comparisons test, compared to GIST48B in the same treatment condition: ***P<0.001: **, P<0.01. Taken together, these data show a dependency in GIST, and across select cell lines in DepMap, for the Menin-MLL complex in the maintenance of the GIST epigenome.

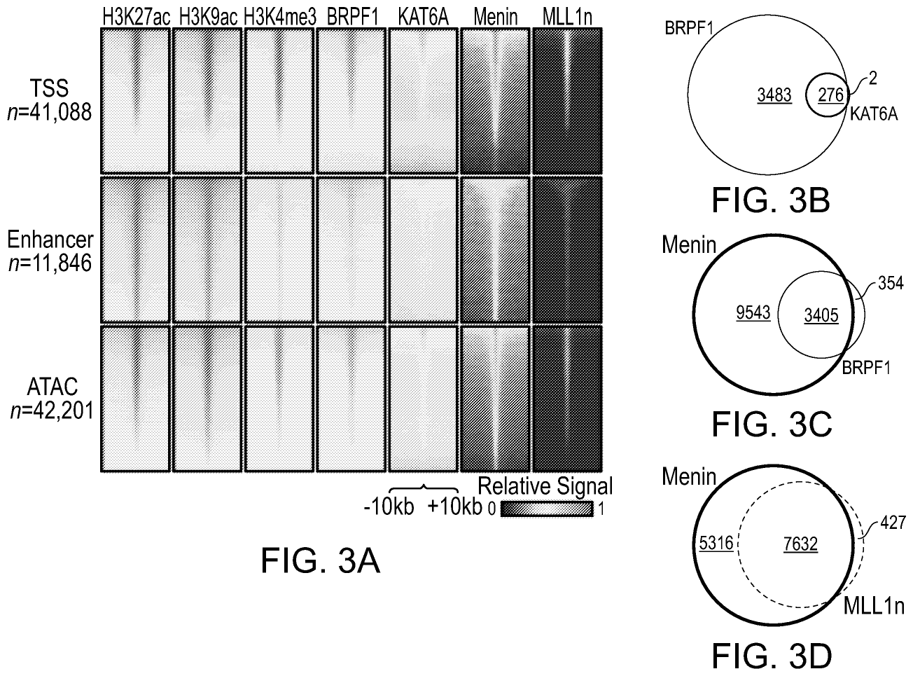

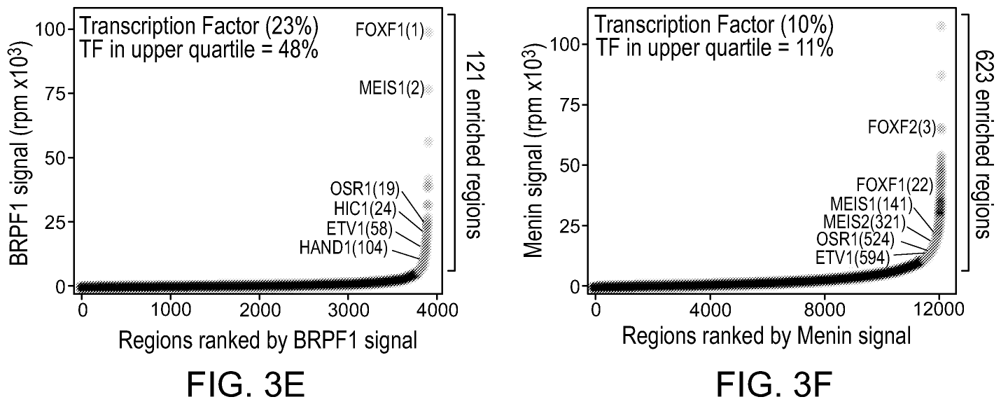

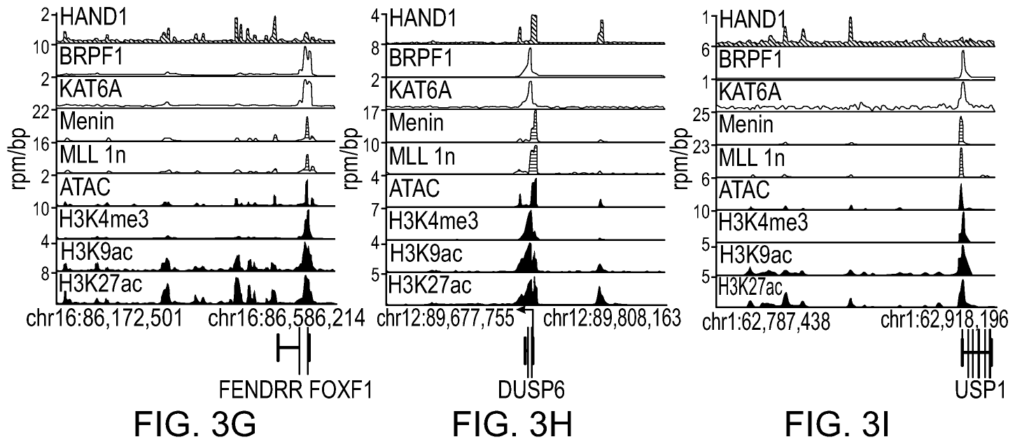

ChIP-seq for histone marks H3K4me3, BRPF1, and KATA6 was performed to define where in the GIST genome the Menin-MLL complex binds and acetylates histones. Genomic regions of binding of Menin and MLL1 were identified using the analogous method CUT&Tag (Kaya-Okur et al., Nat. Commun. 10 (1): 1930 (2019)). Menin-MLL complex members were found to be localized at the transcriptional start sequences (TSSs) of active genes, as determined by their co-occupancy with H3K27ac and H3K9ac, see FIG. 3A top row. In contrast, very little occupancy of these chromatin complex members at H3K27ac-defined enhancers were observed, see FIG. 3A middle row. ATAC peaks, which include accessible DNA sites at both TSSs and enhancers, showed an intermediate level of Menin-MLL complex binding, see FIG. 3A bottom row. In FIG. 3A scaled read densities+10 kb from the TSS, H3K27ac-defined super enhancers or ATAC-defined peaks are shown in rows.

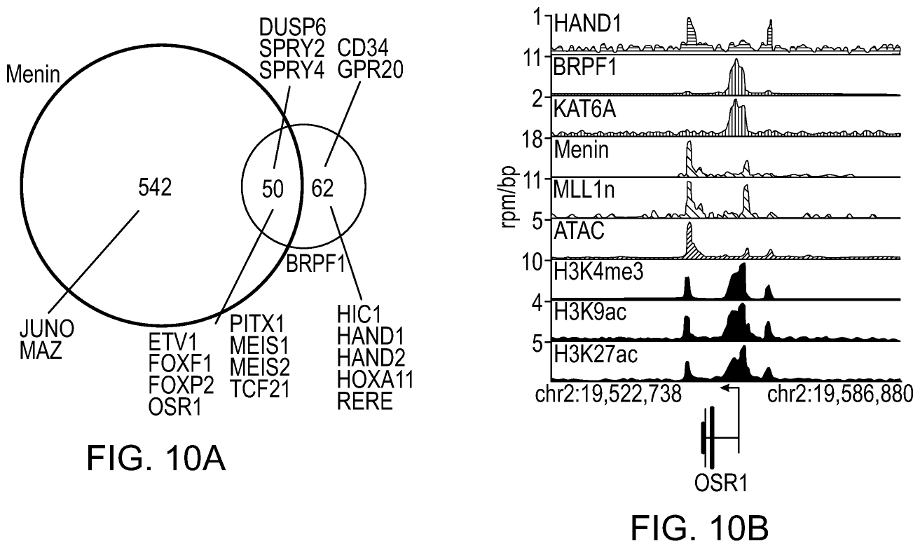

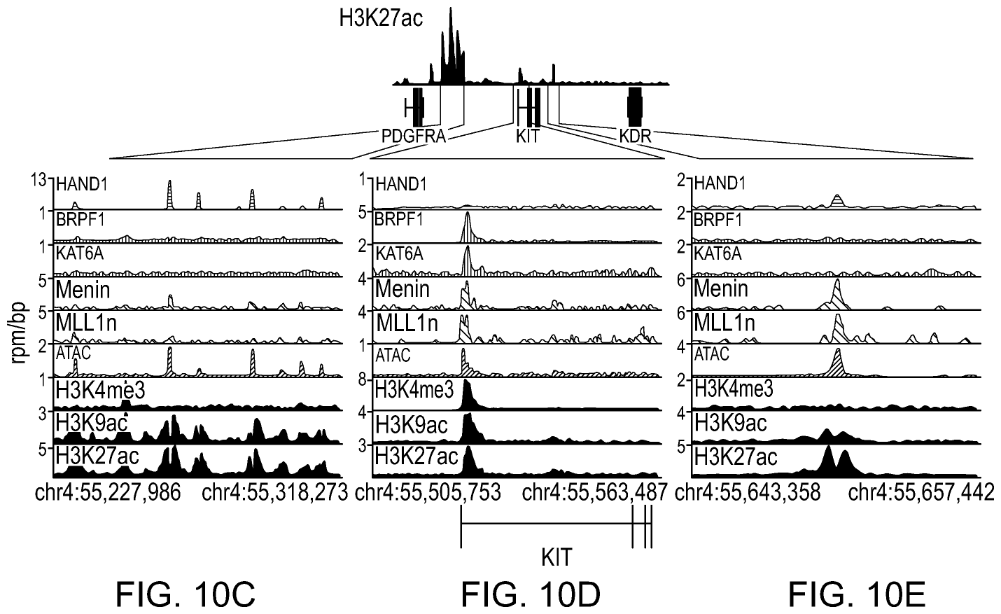

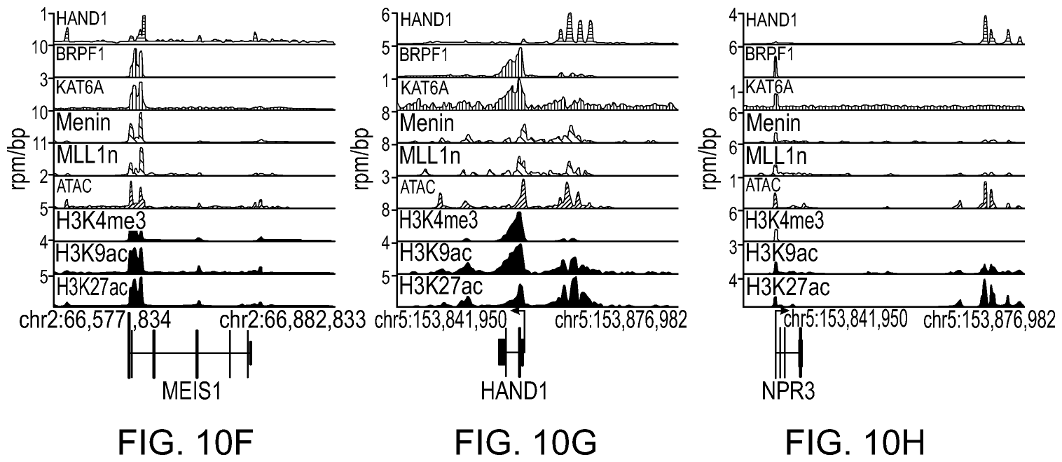

Next the genomic regions that displayed strong enrichment for Menin in the ChIP-seq and CUT&Tag datasets were analyzed, reasoning that these factors are representative of Menin-MLL complex. While this protein binds to thousands of sites genome-wide. disproportionate enrichment was seen in 3-5% of these genomic regions, many of which have clear relevance to GIST, as illustrated in FIGS. 3E, and 10A. The percent of all enriched regions associated with TF genes and those in the upper quartile are indicated in FIG. 3E and FIG. 3F. Transcription factor sites (TFs), particularly those among the group of core and accessory GIST TFs (Hemming et al., Proc. Natl. Acad. Sci. U.S.A 115 (25):E5746-E5755 (2018). Hemming et al., Cancer Res. 79 (5): 994-1009 (2019)), were included in these enriched regions, as were negative regulators of KIT signaling from DUSP and sprouty families and genes used as biomarkers for GIST (e.g. GPR20. CD34 (Corless et al., J. Clin. Oncol. 22 (18): 3813-25 (2004))). ChIP-seq and CUT&Tag tracks show binding of Menin-MLL complex members at the TSS and gene body of these enriched genes, analogous to H3K4me3, such as at core TF members. DUSP6 and the essential gene USP1, as illustrated in FIGS. 3F-3H, and 10B; by contrast. H3K27ac and H3K9ac are enriched at both enhancer regions and gene bodies, and the GIST accessory TF HAND1 binds exclusively to enhancers. Menin-MLL complex members were notably not enriched at the KIT locus. though there was evidence that these regulators bind to the TSS and a region downstream of the gene body. see FIG. 10C. Maximal binding of Menin-MLL complex members within and immediately downstream of the TSS of other regions of enrichment was seen, with detectable signal evident at some enhancers of these highly regulated genes, see FIGS. 10D-10H. These data indicate that the Menin-MLL complex is globally present at active genes, with enrichment at a subset of genes relevant to the GIST transcriptional program.

### Example 3: Menin Inhibition Disrupts GIST Cell Proliferation without Apoptosis

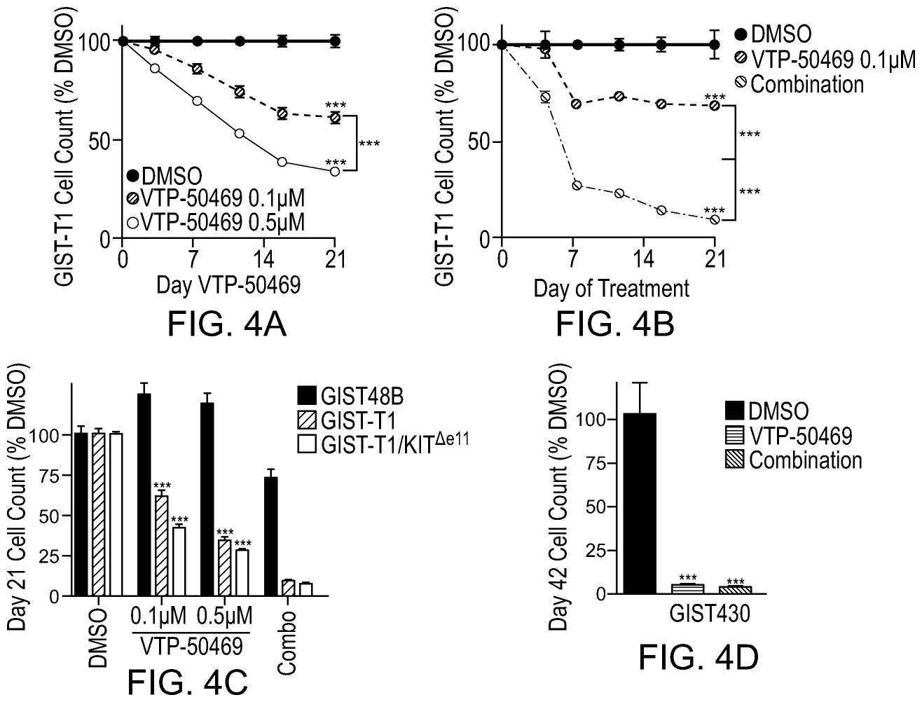

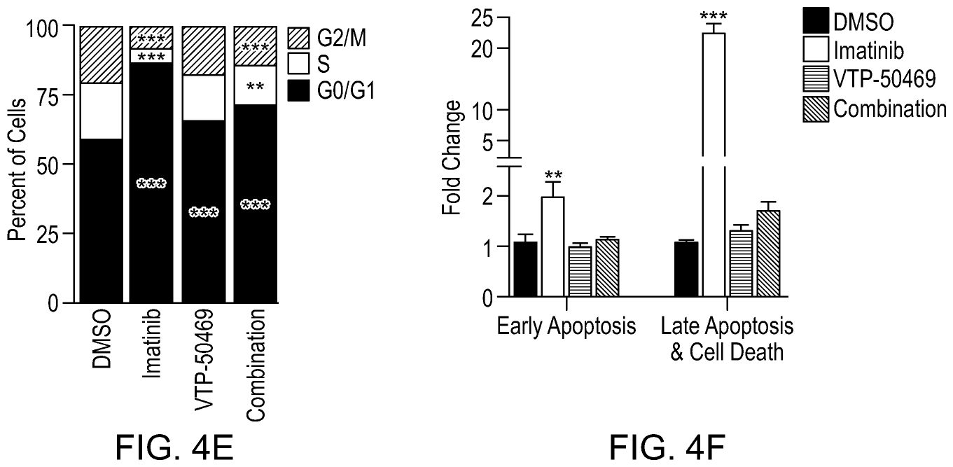

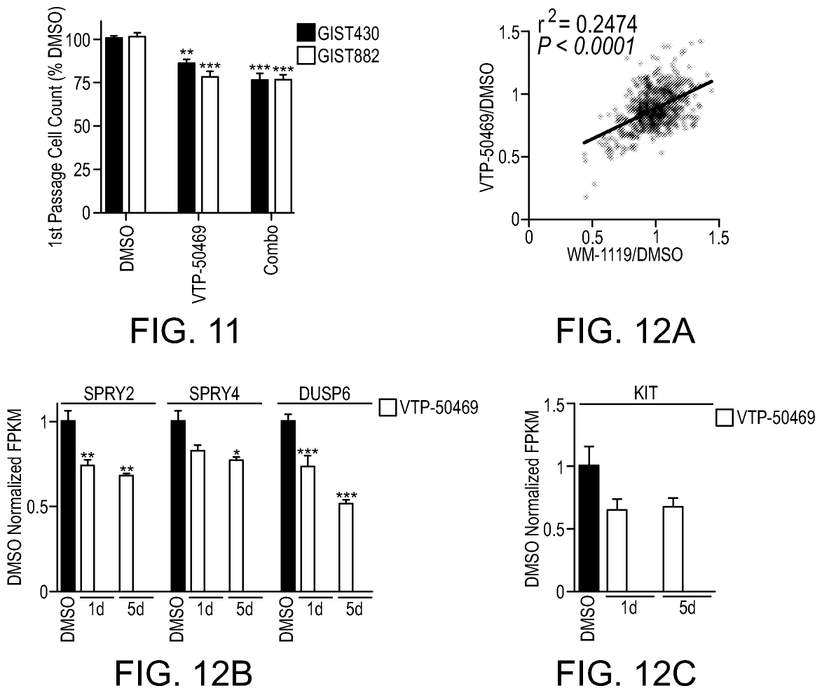

Based on the genetic data and genomic colocalization of the Menin-MLL complex. it was reasoned that small molecule inhibitors targeting GIST-linked complexes would be a viable therapeutic approach. To explore the functional consequences of Menin-MLL disruption. GIST-T1 cells were treated with the Menin inhibitor VTP-50469 (Krivtsov et al., Cancer Cell 36:660-673 (2019)), alone or in combination with the selective KAT6A inhibitor WM-1119 (Baell et al., Nature 560 (7717): 253-257 (2018)). At sub-micromolar concentrations. VTP-50469 decreased GIST cell proliferation in a growth over time assay, as illustrated in FIGS. 4A-4B, with a greater effect seen with combination of the two drugs (FIG. 4B). Demonstrating selective toxicity of these inhibitors in KIT-dependent GIST cell lines, the KIT-independent GIST48B cell line showed modest or no alterations in proliferation after a 21-day drug treatment (FIG. 4C). in agreement with genetic data from CRISPR experiments (FIG. 2I). Previous studies of the KIT enhancer used an sgRNA directed at the KIT TSS to ablate endogenous KIT expression while simultaneously rescuing cell viability by expressing a viral promoter-driven KIT construct bearing the same activating mutation (Hemming et al., Cancer Res. 79 (5): 994-1009 (2019)). This KIT-dependent KIT rescue cell line was similarly susceptible to VTP-50469 alone or in combination with WM-1119, as illustrated in FIG. 4C. indicating that regulation of the endogenous KIT locus is not the principal mechanism of toxicity of these compounds. Statistical comparisons reference GIST48B under the identical treatment are shown in FIG. 4C. To confirm the proliferative effects of these inhibitors in additional GIST cell lines, the slower growing KIT mutant cell lines GIST430 (Hemming et al., Cancer Res. 79 (5): 994-1009 (2019)) and GIST882 were treated with VTP-50469 alone or in combination with WM-1119, observing analogous anti-proliferative effects arising from drug treatment, as illustrated in FIGS. 4D and 11A. The data in FIG. 11 were analyzed by one-way ANOVA with Dunnett's multiple comparisons test: compared to DMSO control *** P<0.001; **, P<0.01; *, P<0.05.

To evaluate the cellular phenotypic consequences of VTP-50469 treatment, cell cycle and apoptosis assays were preformed utilizing VTP-50469 and the TKI imatinib as comparator. While imatinib acutely and potently caused G0/G1 cell cycle arrest within 72 hours, eight days of treatment with VTP-50469 lead to a modest increase in the fraction of cells in G0/G1, as illustrated in FIG. 4E; the combination of VTP-50469 with WM-1119 led to more marked disruption of the cell cycle after an 8-day treatment (FIG. 4E). While 72-hour imatinib treatment produced a significant increase in early and late-phase apoptosis and cell death, an 8 day treatment of VTP-50469 alone or in combination with WM-1119 did not significantly increase apoptosis or cell death compared to DMSO control, as illustrated in FIG. 4F, where n=3 to 5 per condition. Data were analyzed by two-way or one-way ANOVA, where appropriate, with Tukey's post-hoc test, compared to DMSO or the indicated condition: *** P<0.001: **, P<0.01. Taken together, these data show that the Menin-MLL complex is targetable and presents a unique vulnerability in GIST, and in keeping with its distribution across the genome at TSSs of active genes, that it plays an essential role in gene regulation greater than just KIT gene expression. Disruption of this complex alone or in combination with disruption of the MOZ complex causes alterations in the cell cycle but not in programmed cell death.

### Example 4: Menin-MLL Inhibition Causes Global Gene Expression Changes

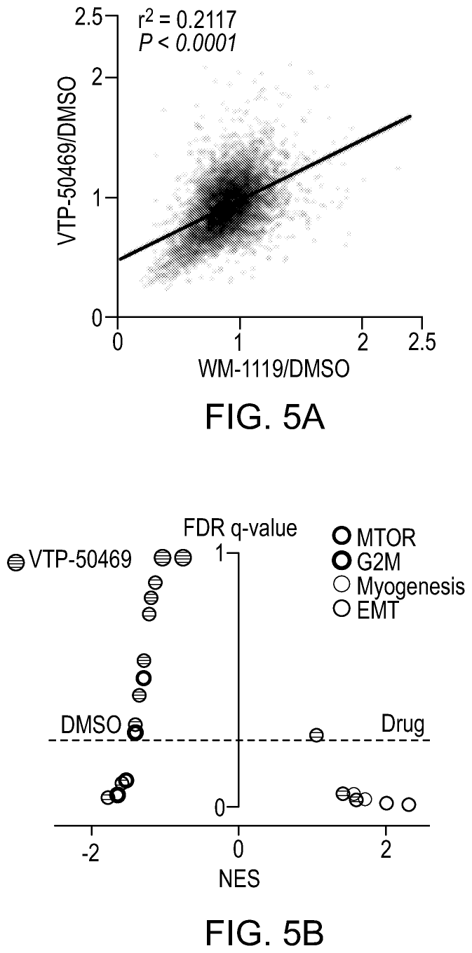

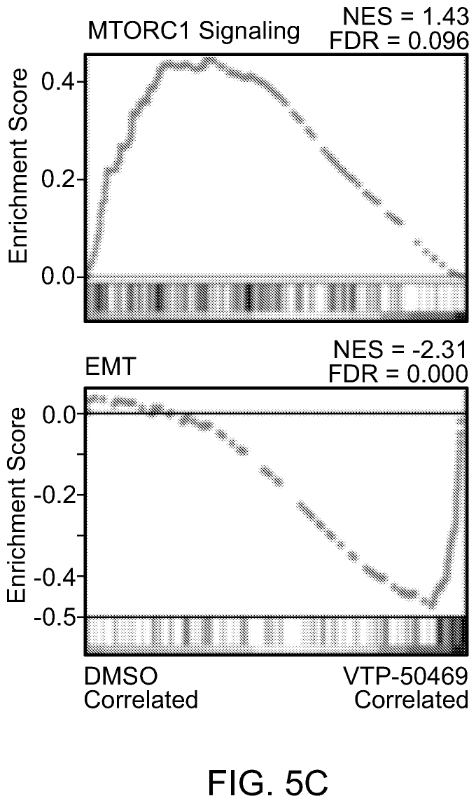

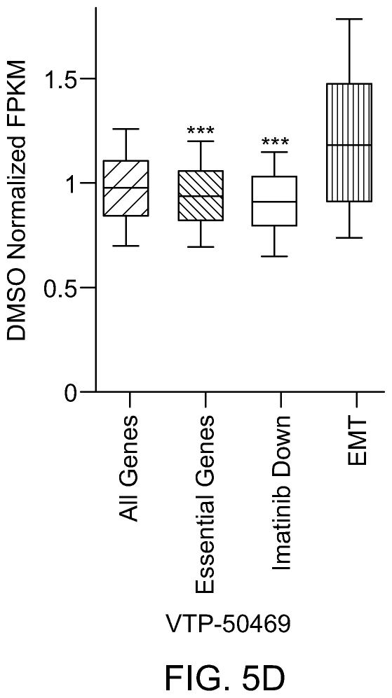

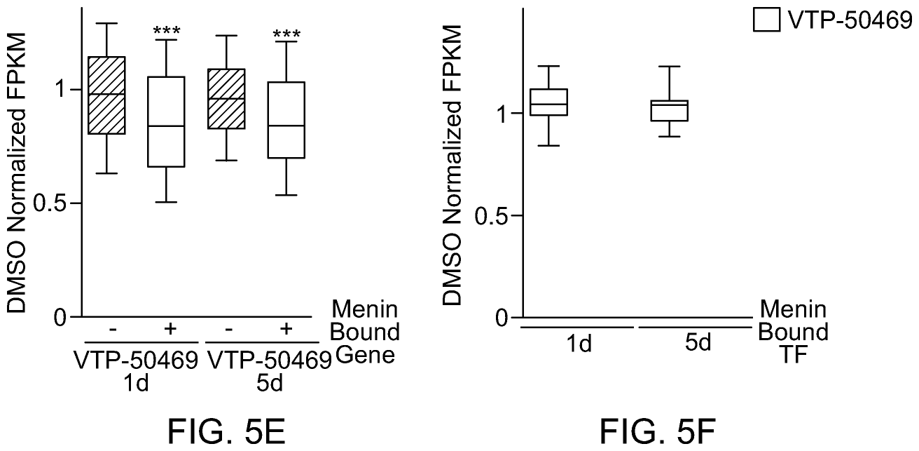

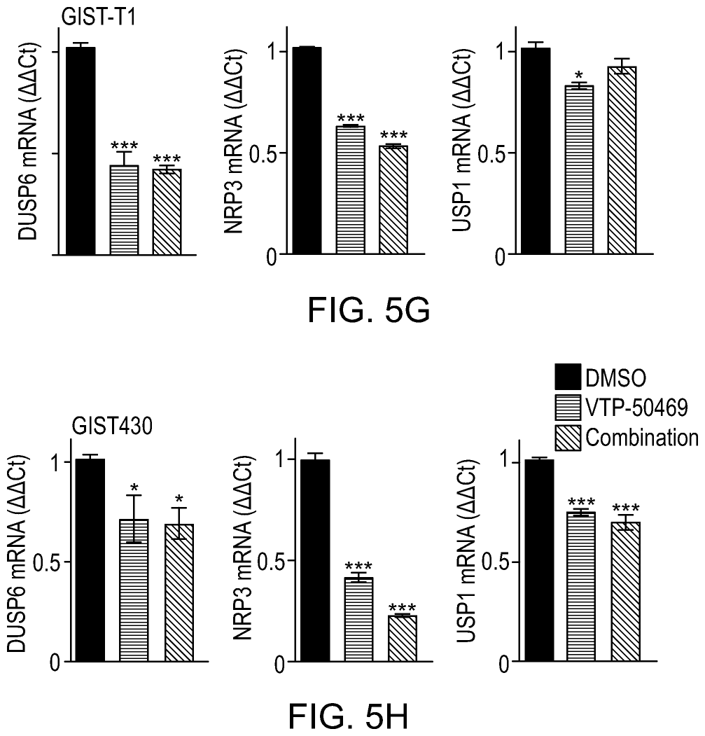

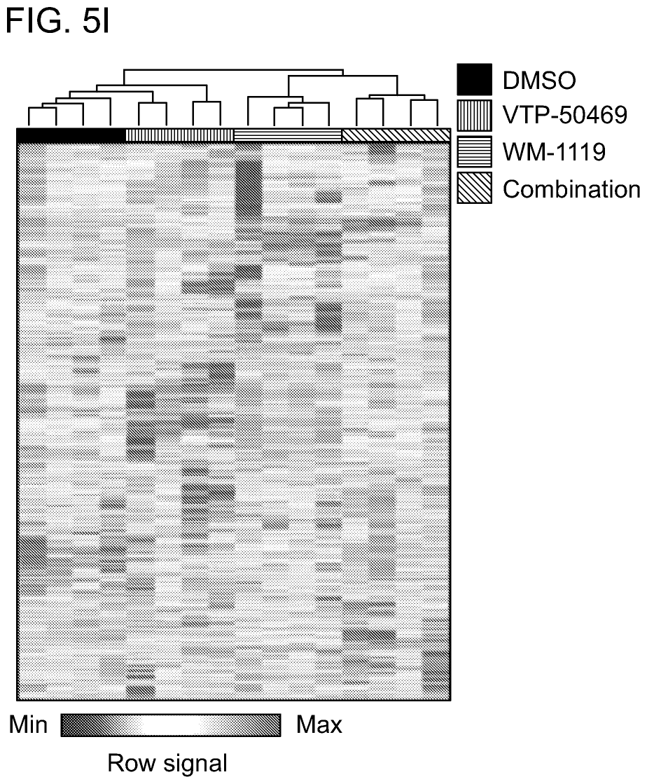

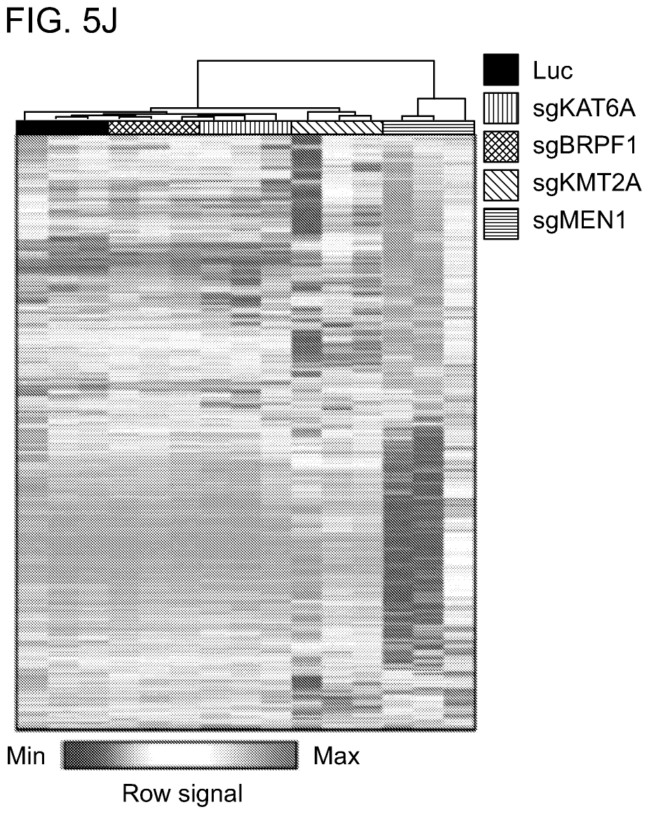

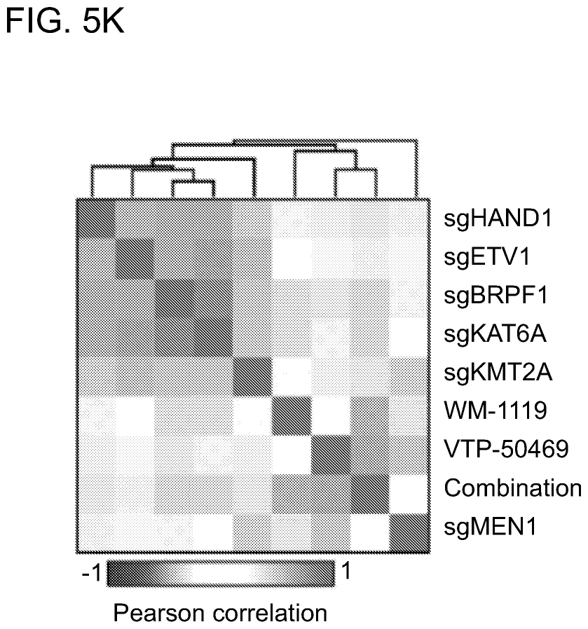

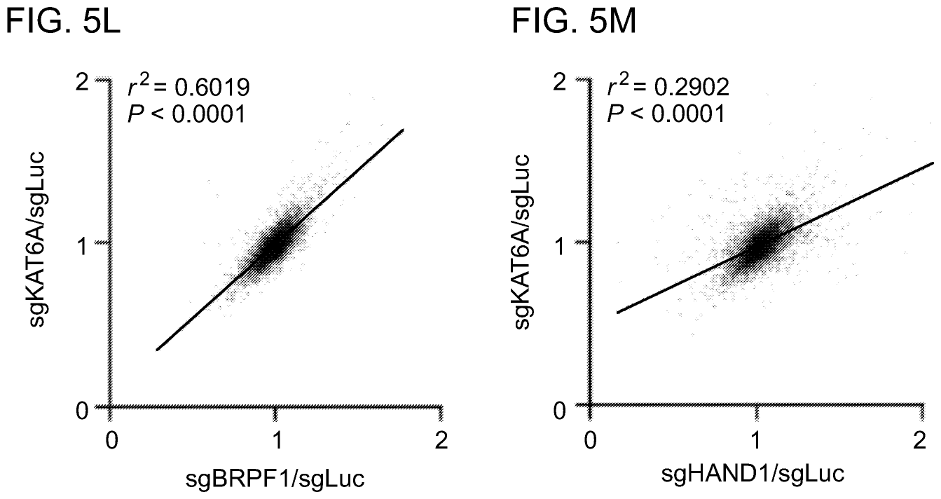

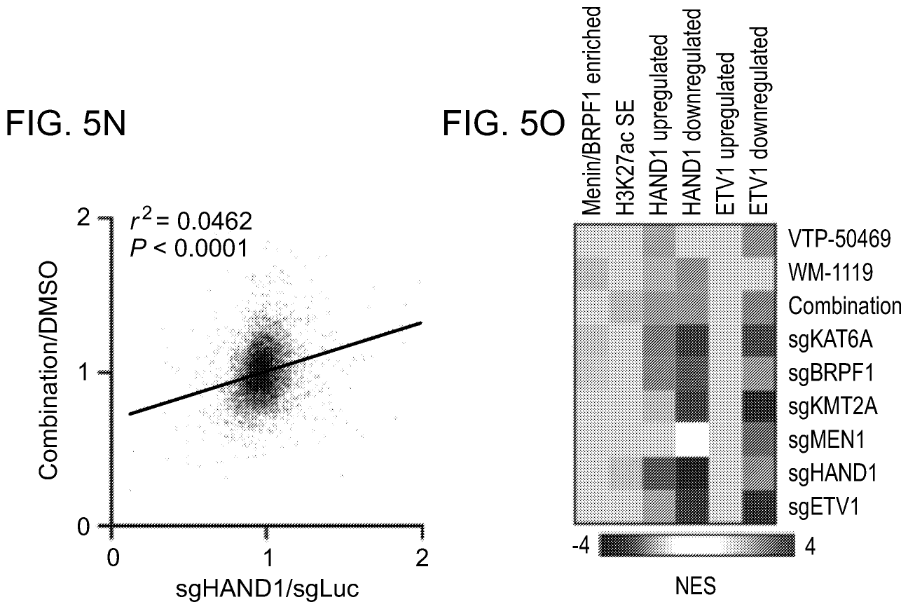

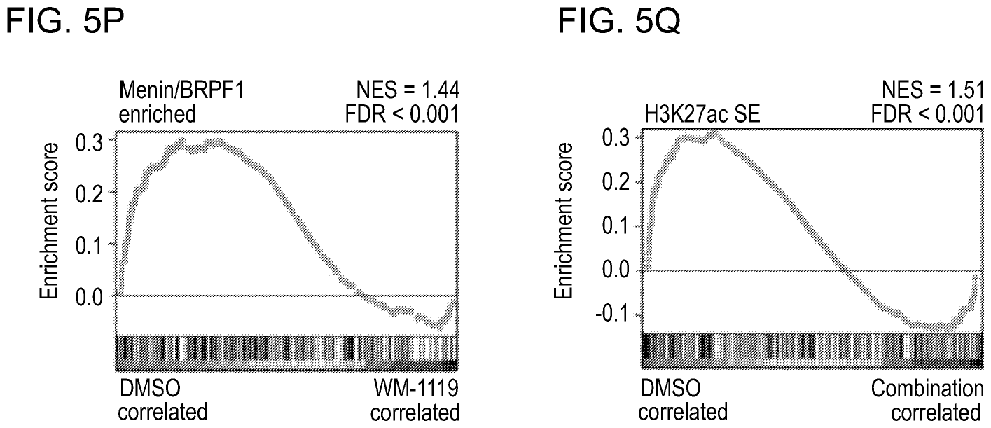

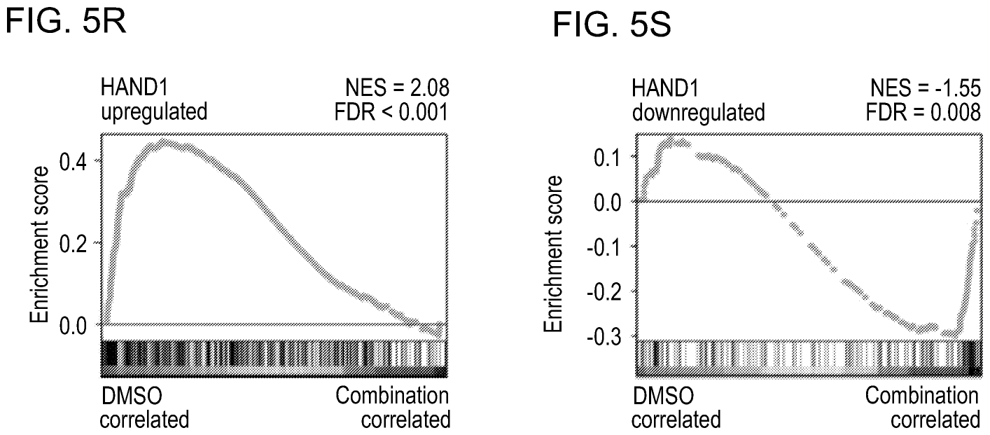

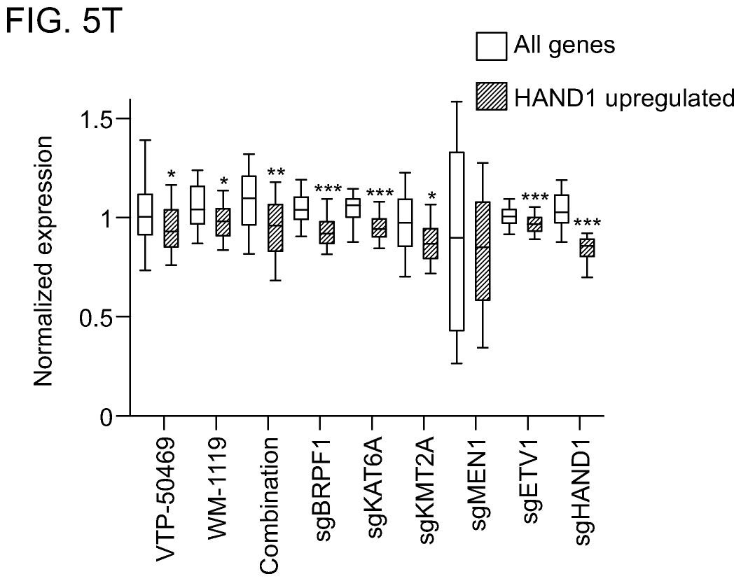

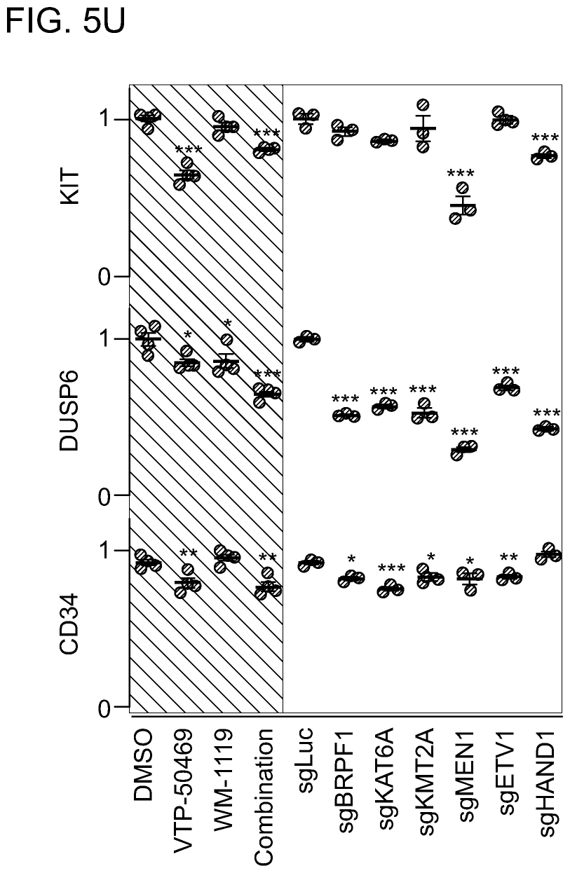

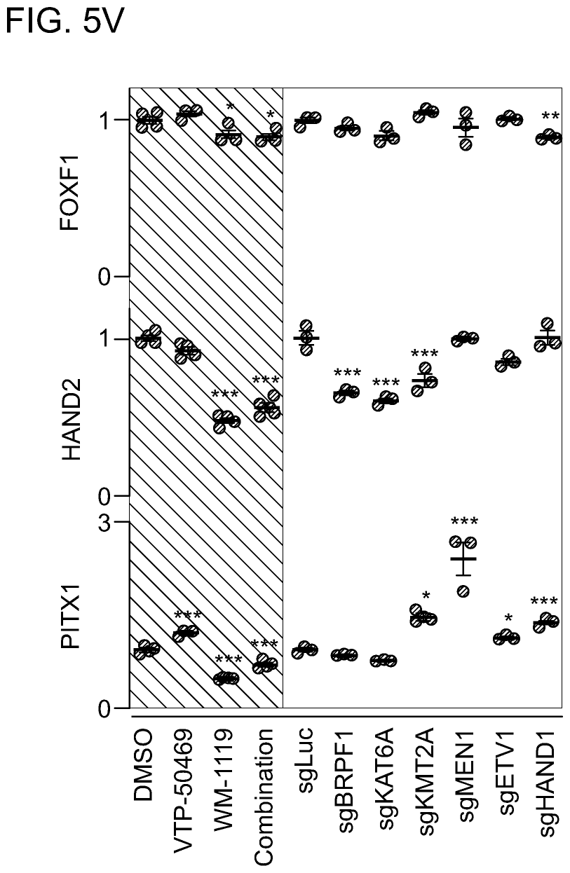

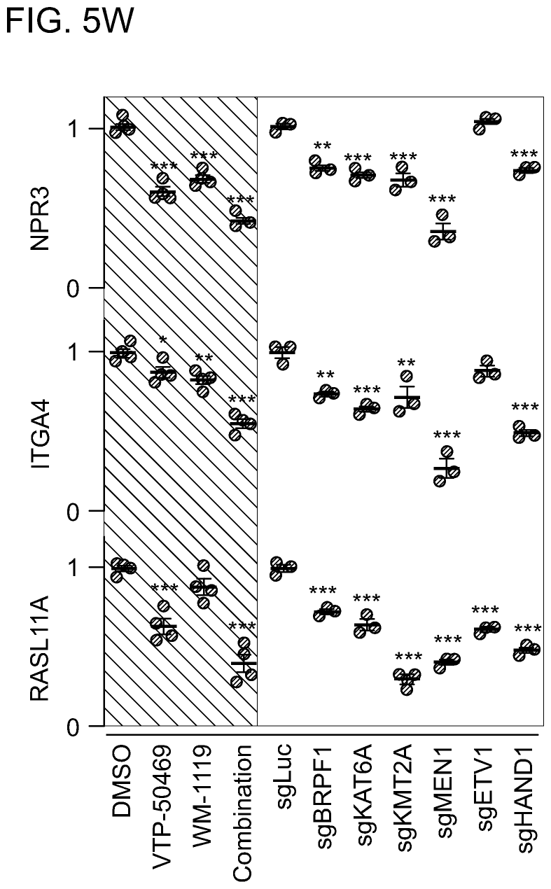

Observing the genome-wide occupancy of Menin-MLL complexes, with select regions of enrichment, it was next probed for selective changes in gene expression arising from VTP-50469, WM-1119, and combined VTP-50469 and WM-1119 treatment to further detail the growth inhibitory phenotype arising from their targeted disruption (FIG. 5A, with the Person correlation shown). RNA-seq on GIST-T1 cells treated with VTP-50469 or WM-1119 for 1 and 5 days was preformed, comparing to DMSO treatment as control. VTP-50469 and WM-1119 treatment modestly altered expression of numerous genes (n=5,095 of FPKM>10), with significant correlation of gene expression changes between these inhibitors, with the VTP-50469 and combination groups showing the greatest deviation from control (FIG. 5A and FIG. 5I). FIG. 5I illustrates the unsupervised hierarchical clustering of RNA-seq data comparing the 5-day treatment of GIST-T1 with VTP-50469 at 0.5 μmol/L. WM-1119 at 1 μmol/L. or the combination with each drug at 0.1 μmol/L (n=4 per condition) for all expressed genes (>10) fragments per kilobase of transcript per million mapped reads (FPKM). n=7.106).

The gene expression changes following genetic disruption of MOZ and Menin-MLL complexes using sgRNAs targeting two complex members each was also evaluated. Using this genetic system. sgRNAs targeting MEN1 led to global changes in gene expression, with disruption of other MOZ and Menin-MLL1 complex members showing less dramatic changes (FIG. 5J), which illustrates the unsupervised hierarchical clustering of RNA-seq data comparing GIST-T1/Cas9 cells transduced with sgRNAs targeting KAT6A. BRPF1. KMT2A, MEN1, or luciferase as control (n=3 per condition) and collected at day 5.

To integrate and compare transcriptional changes arising from either pharmacologic or genetic disruption. the correlation of gene expression changes from control across the transcriptome was evaluated. For comparison. the transcriptional changes from sgRNA-mediated disruption of the GIST TFs HAND1 and ETV1 were also evaluated. sgRNAs targeting MOZ complex members KAT6A and BRPF1 showed the highest degree of correlation and. moreover, induced similar global transcriptional changes, as did the disruption of HAND1. ETV1, or KMT2A (FIG. 5K-FIG. 5S). Pharmacologic inhibition of MOZ and/or Menin-MLL complexes had a comparatively weak but positive correlation with sgRNAs targeting GIST TFs. MOZ complex members, and KMT2A, whereas genetic disruption of MEN1 showed the least correlation with other conditions (FIG. 5K and FIG. 5N). FIG. 5K shows a Pearson correlation of control-normalized RNA-seq data from FIG. 5I and FIG. 5J with the inclusion of control-normalized RNA-seq data from GIST-T1/Cas9 cells transduced with sgRNAs targeting HAND1 and ETV1. These data demonstrate disparate global transcriptional consequences arising from genetic or chemical disruption of these epigenetic regulators.

Using gene set enrichment analysis (GSEA). the expression of genes enriched for MOZ and Menin-MLL complexes. H3K27ac-defined superenhancer (SE)-associated genes. and genes regulated by TFs HAND1 and ETV1 were evaluated. Among these GIST-associated gene lists. genes regulated by TFs were the most markedly affected, with drug or sgRNA treatment causing reduced expression of genes upregulated by HAND1 and increased expression of genes normally downregulated by HAND1 or ETV1 function (FIG. 5O), which shows normalized enrichment scores (NES) from GSEA gene sets including genes showing enrichment for Menin and BRPF1 (n=385), GIST-T1 SE-associated genes defined by H3K27ac (n=366), genes upregulated (n=421) or downregulated (n=165) by HAND1, and genes upregulated (n=438) or downregulated (n=31) by ETV1.

Only gene sets with significant FDRs are displayed using the color scale, with those bearing nonsignificant FDRs indicated in gray. Genetic or pharmacologic MOZ disruption exhibited the greatest effect on genes bound by Menin or BRPF1 (FIG. 5H), while only targeting HAND1 or the combination of WM-1119 and VTP-50469 decreased the expression of SE-associated genes (FIG. 5Q). However, common to all conditions was the disruption of HAND1-associated gene expression, with expression values of MOZ and Menin-MLL complex disruption using either inhibitors or sgRNAs phenocopying direct HAND1 knockout (FIG. 5R-FIG. 5T).

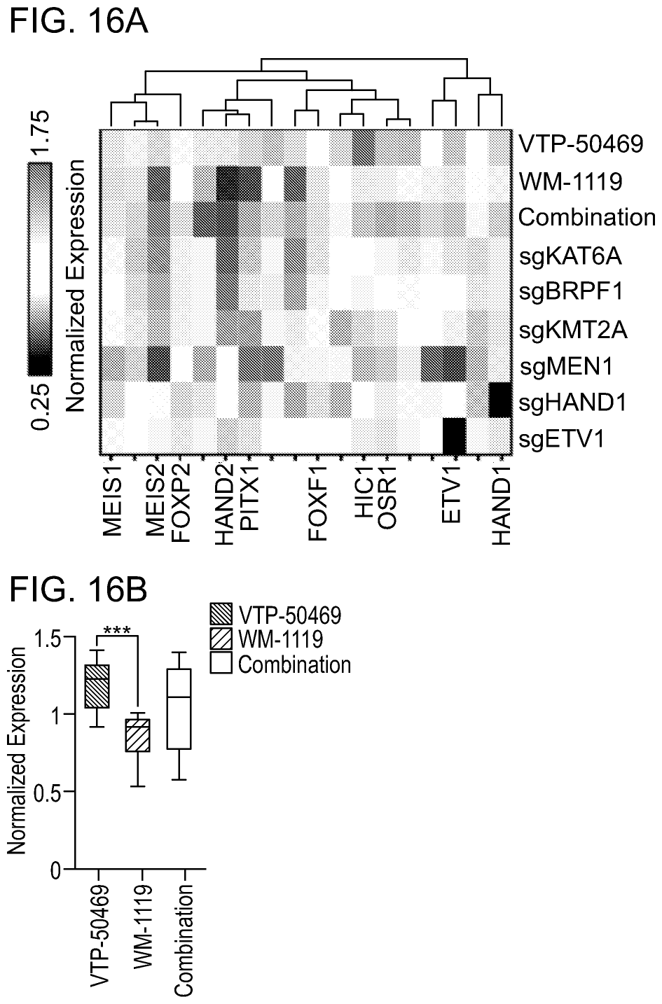

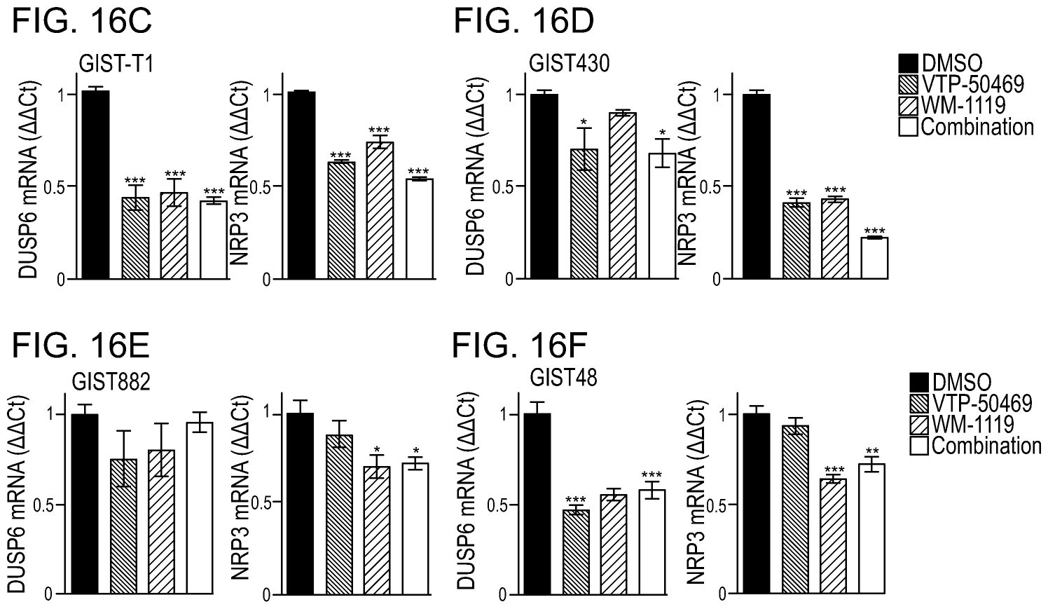

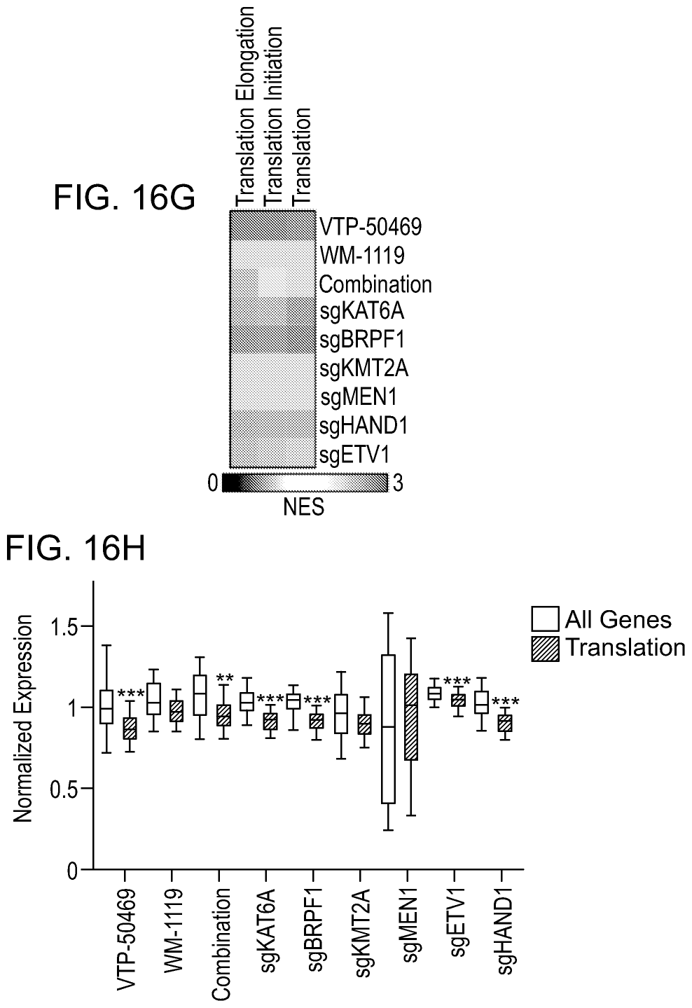

KIT gene expression was most markedly affected by pharmacologic or genetic disruption of Menin, whereas there was a common loss of DUSP6, a negative regulator of KIT signaling, and the GIST biomarker CD34 (FIG. 5U). Expression of several core GIST TFs was altered by genetic or pharmacologic MOZ or Menin-MLL complex disruption-most notably FOXF1, HAND2, and PITX1—with WM-1119 exerting the greatest global reduction in TF expression (FIG. 5V, FIG. 16A-FIG. 16B). Several other genes highly regulated by HAND1 expression, including NPR3, ITGA4, and RASL11A, showed similar loss of expression with disruption of MOZ and Menin-MLL complexes (FIG. 5W). Comparable reductions in gene expression of DUSP6 and NPR3 were seen in the KIT-dependent GIST cell lines GIST430, GIST882, and GIST48 by qRT-PCR (FIG. 16C-FIG. 16F).

Among all Reactome gene sets, processes related to protein translation were the most recurrently altered gene sets among treatment conditions, with most drug and sgRNA data sets showing a decrease in gene expression (FIG. 16G-FIG. 16H). Taken together, these results show that both genetic and pharmacologic means of MOZ and Menin-MLL complex disruption lead to selective alteration of transcriptional programs associated with GIST TFs. and most remarkably HAND1. Further, dual inhibition of Menin and MOZ with small molecules induces complementary effects on global gene expression and reduces expression of GIST SE-associated genes.

GSEA (Subramanian et al., Proc. Natl. Acad. Sci. USA 102:15545-50 (2005)) was used to explore pathway alterations associated with drug treatment. VTP-50469 treatment led to similar changes in Hallmark gene sets, with significant upregulation of gene sets associated with myogenesis and epithelial mesenchymal transition (EMT): drug treatment also led to reductions of expression of gene sets associated with cell cycle and mitogenic signaling, as illustrated in FIGS. 5B and 5C, with the MTORC1 Signaling. G2M Checkpoint, Myogenesis and EMT gene sets are indicated on the figure for each condition. While there were minimal changes with drug treatment on the global average of gene expression, GIST-relevant gene sets showed significant changes. Genes identified as essential for GIST (see FIGS. 1C and 12A) and genes downregulated after 6 hours of imatinib treatment (Hemming et al., Cancer Res. 79 (5): 994-1009 (2019)) showed corresponding reductions in expression arising from VTP-50469 treatment, with the Hallmark EMT signature showing upregulation as comparator, as illustrated in FIG. 5D (all expressed genes n=5,093, essential genes n=1,507, genes downregulated >2.5-fold by 6 hours imatinib treatment n=544, and Hallmark EMT genes n=63). Data were analyzed by one-way ANOVA with Dunnett's multiple comparisons test, compared to DMSO: ***, P<0.001: **, P<0.01: *, P<0.05. FIG. 12A shows the ratio of expression between inhibitor and DMSO treatment for the top 500 essential genes following 5 days of inhibitor treatment, and the Pearson correlation is shown. Genes with disproportionately higher loading with Menin (n=294) showed greater reductions in gene expression following inhibitor treatment at both 1 and 5 days (as compared to genes lacking enrichment, n=4,799), as illustrated in FIG. 5E. Among DUSP and sprouty family members highly expressed in GIST, DUSP6 was the most significantly reduced, while KIT expression showed only a trend towards reduced expression by VTP-50469 by 5 days, as illustrated in FIGS. 12B-12C. These reductions were confirmed in the select transcripts DUSP6, NPR3 and USP1 in GIST-T1 and GIST430 arising from VTP-50469 alone or in combination with WM-1119, using qRT-PCR, as illustrated in FIGS. 5G-5H, with the combination treatment notably showing no robust additive effect on gene expression of these targets. For FIGS. 5G-5H, and 12B-12C n=3 to 4 per group and data were analyzed by one-way ANOVA with Dunnett's multiple comparisons test, compared to DMSO: ***, P<0.001: **, P<0.01: *, P<0.05. Together with the chromatin studies, these data highlight the pathway-selective changes in gene expression associated with pharmacologic Menin inhibition, alone or in combination with MOZ inhibition, identifying alterations in genes related to cell cycle, viability, trophic KIT signaling, and potentially altering differentiation state by activating mesenchymal developmental programs.

### Example 5: Disruption of Chromatin and Transcriptional Regulatory Protein Interactions with Menin Inhibition

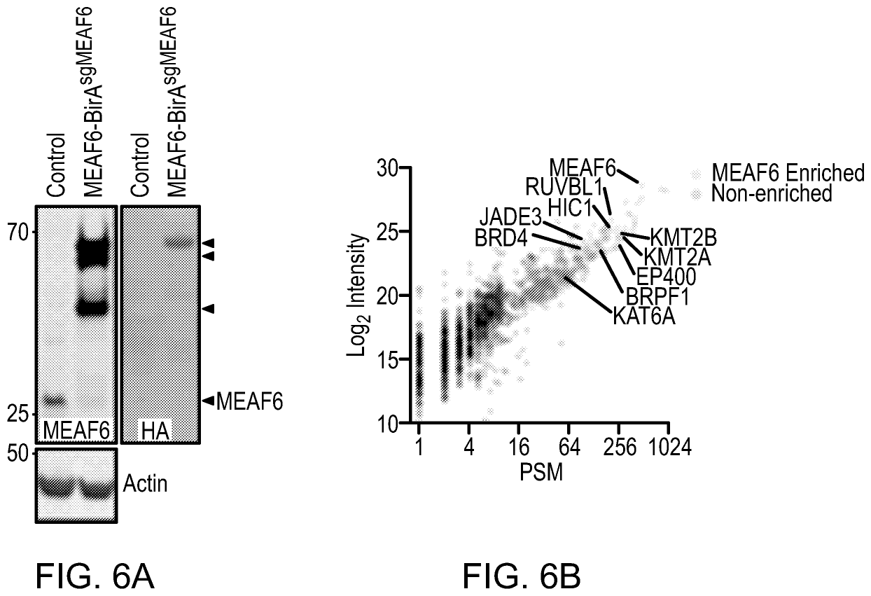

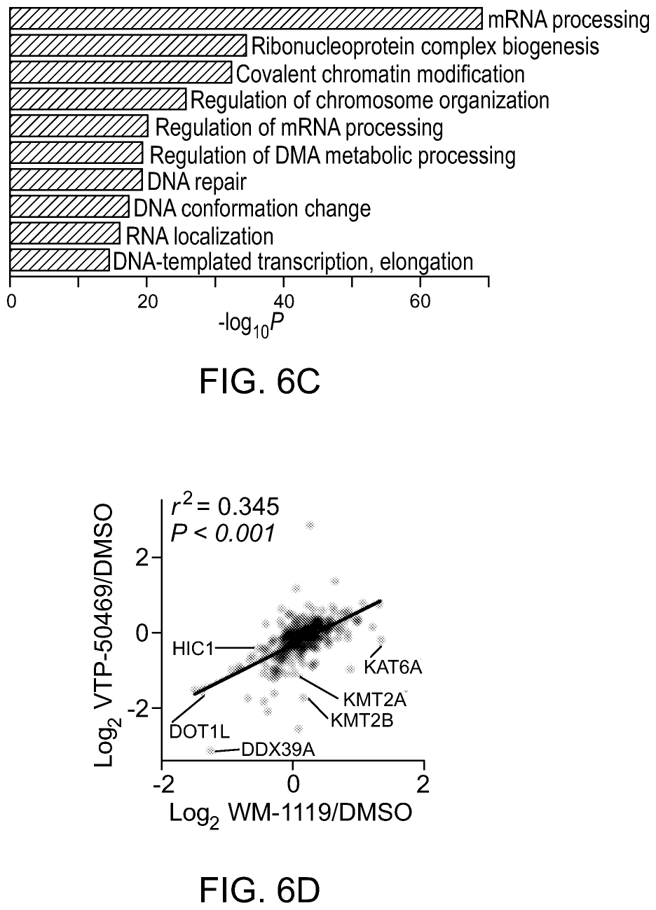

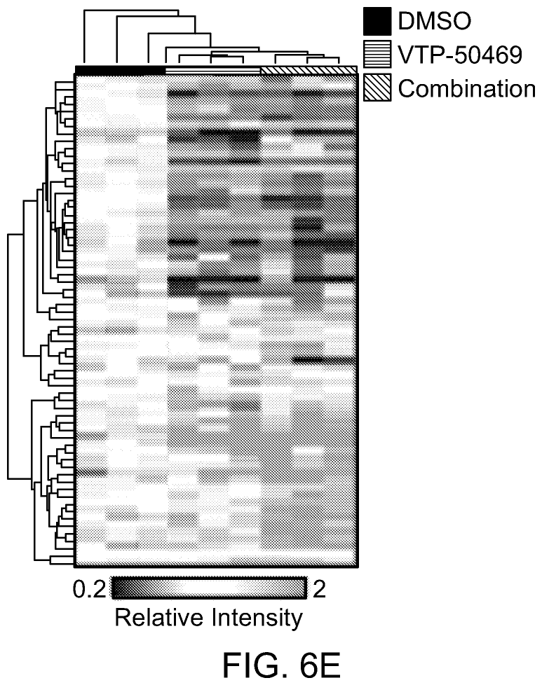

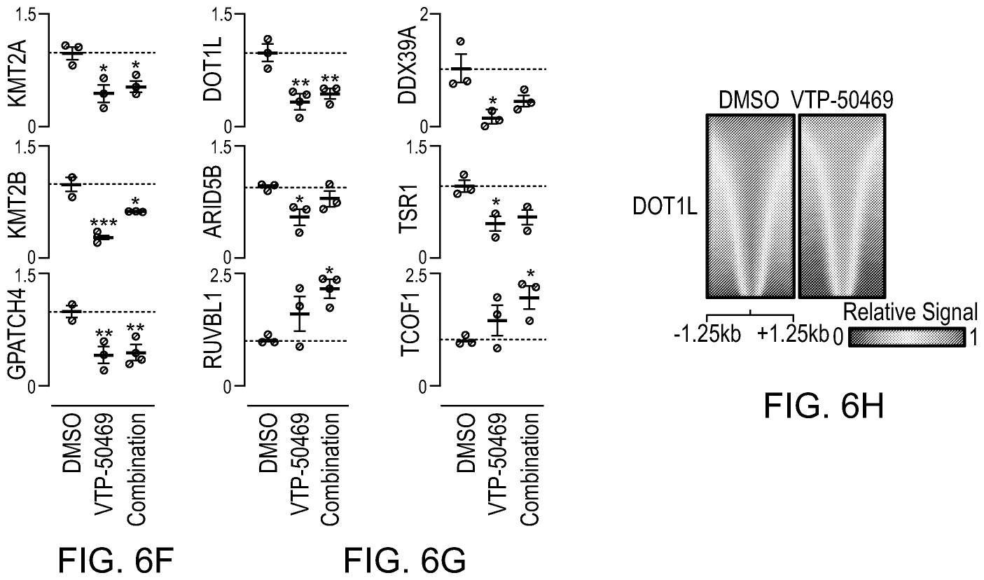

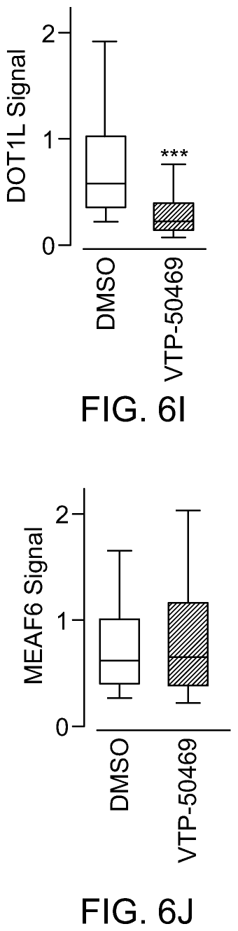

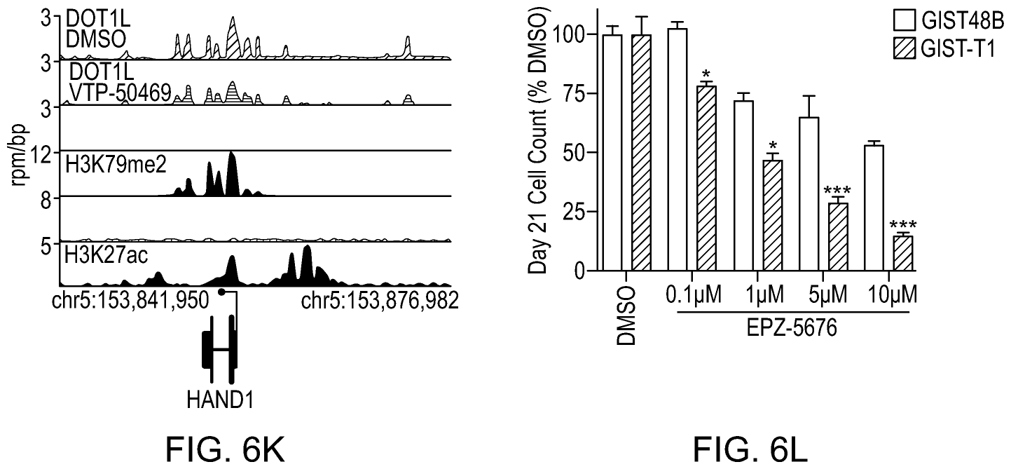

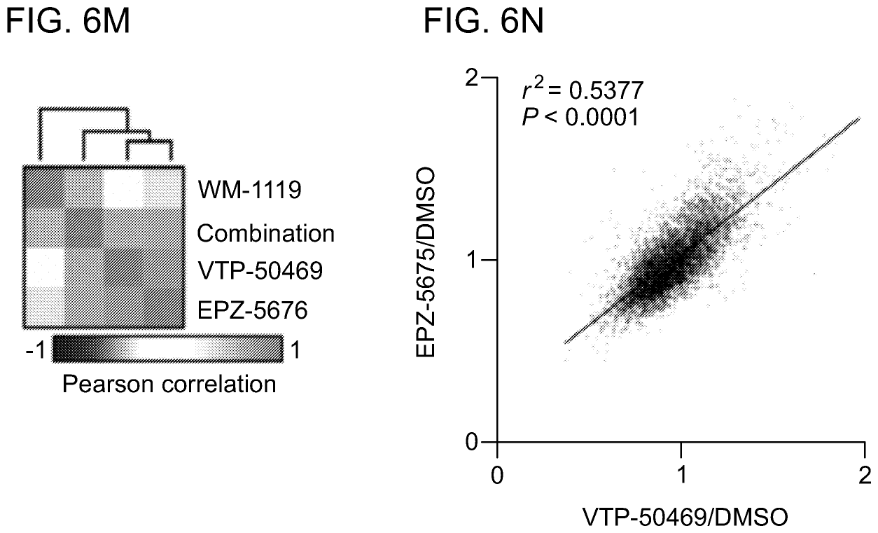

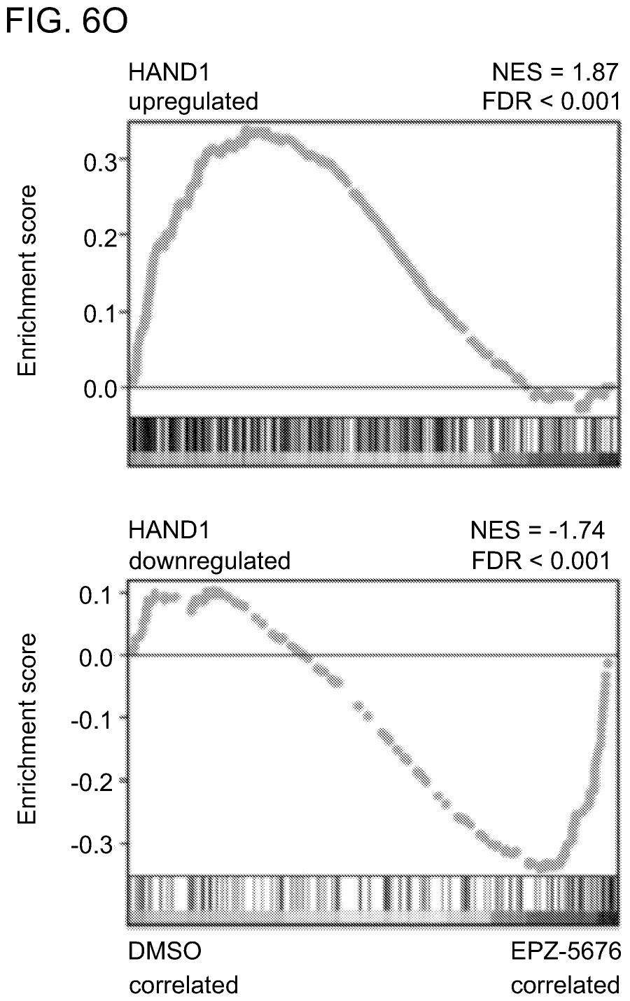

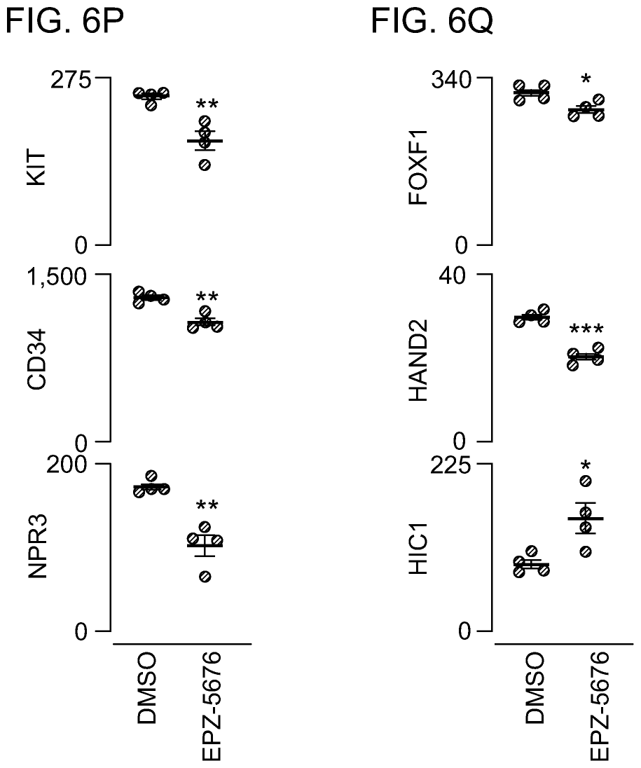

As MOZ and Menin-MLL complexes function in a coordinated fashion at highly regulated genomic regions together with other chromatin regulators, the effects on local protein interactions were evaluated in the presence or absence of VTP-50469 alone or in combination with WM-1119. The BioID system (Lambert et al., J. Proteomics 118:81-94 (2015)) was used to append the biotin ligase BirA* to the N-terminus of the MOZ complex member MEAF6, which enabled the covalent labeling of proteins localized within 10 nm with a biotin moiety. To ensure appropriate incorporation if the BirA*-tagged MEAF6 into the MOZ complex, the CRISPR/Cas9 and a MEAF6-targeted sgRNA to disrupt the endogenous MEAF6 was used, which would otherwise be lethal if not functionally replaced by the stably expressed MEAF6-BirA construct (FIG. 2E). Stable expression of the N-terminally tagged MEAF6-BirA in GIST-T1 to led to high levels of protein production, with evidence of N-terminal degradation products observed on Western blot, including MEAF6 and HA, indicating the endogenous and full-length rescue construct, or actin as loading control (FIG. 6A). A construct that fused BirA* to the DNA binding domain of IKZF1, which retained its nuclear localization signal was used as a nuclear background BioID control. Following labeling of cells for 24 hours with biotin, a streptavidin pulldown followed by mass spectrometry was performed, identifying 243 proteins labeled by MEAF6-BirA and enriched above control (FIG. 6B, Table 4) In FIG. 6B, MEAF6-enriched proteins, indicated in blue, show >2-fold intensity enrichment compared to background control (n=243). Select interactors are labeled. Labeled interactors included chromatin regulatory proteins such as KMT2A/MLL1, KMT2B/MLL2, JADE3 and RUVBL1 in addition to MOZ complex members, enhancer-associated proteins such as BRD4, and the core GIST TF HIC1. Ordering these MOZ-proximal proteins by gene ontology, there was enrichment for cellular processes including DNA repair, mRNA processing and chromatin complex regulation (FIG. 6C). These data demonstrate the integrated cellular function of these transcriptional regulatory proteins and their complex interactions between splicing factors, enhancers and chromatin complexes.

TABLE 4

| | Gene | | Gene | | Gene |
|---|---|---|---|---|---|
| Reference | Symbol | Reference | Symbol | Reference | Symbol |
| sp\|Q9HAF1\|EAF6_HUMAN | MEAF6 | sp\|Q9UKV3\|ACINU_HUMAN | ACIN1 | sp\|Q96Q89\|KI20B_HUMAN | KIF20B |
| sp\|P46013\|KI67_HUMAN | MKI67 | sp\|Q6UN15\|FIP1_HUMAN | FIP1L1 | sp\|095251\|KAT7_HUMAN | KAT7 |
| sp\|P12270\|TPR_HUMAN | TPR | sp\|Q9NW82\|WDR70_HUMAN | WDR70 | sp\|P61289\|PSME3_HUMAN | PSME3 |
| sp\|P06748\|NPM_HUMAN | NPM1 | sp\|Q9UQR0\|SCML2_HUMAN | SCML2 | sp\|Q9NPF5\|DMAP1_HUMAN | DMAP1 |
| sp\|P19338\|NUCL_HUMAN | NCL | sp\|Q9ULD4\|BRPF3_HUMAN | BRPF3 | sp\|P52272\|HNRPM_HUMAN | HNRNPM |
| sp\|O75182\|SIN3B_HUMAN | SIN3B | sp\|P53999\|TCP4_HUMAN | SUB1 | sp\|Q9H8H0\|NOL11_HUMAN | NOL11 |
| sp\|Q9H307\|PININ_HUMAN | PNN | sp\|O00541\|PESC_HUMAN | PES1 | sp\|P18615\|NELFE_HUMAN | NELFE |
| sp\|Q9Y265\|RUVB1_HUMAN | RUVBL1 | sp\|Q8WXI9\|P66B_HUMAN | GATAD2B | sp\|Q93009\|UBP7_HUMAN | USP7 |
| sp\|P42167\|LAP2B_HUMAN | TMPO | sp\|P09661\|RU2A_HUMAN | SNRPA1 | sp\|Q96JM3\|CHAP1_HUMAN | CHAMP1 |
| sp\|Q7L014\|DDX46_HUMAN | DDX46 | sp\|Q9H583\|HEAT1_HUMAN | HEATR1 | sp\|Q9P2D1\|CHD7_HUMAN | CHD7 |
| sp\|Q13435\|SF3B2_HUMAN | SF3B2 | sp\|Q969G3\|SMCE1_HUMAN | SMARCE1 | sp\|P26358\|DNMT1_HUMAN | DNMT1 |
| sp\|Q13206\|DDX10_HUMAN | DDX10 | sp\|Q86YP4\|P66A_HUMAN | GATAD2A | sp\|Q7Z6E9\|RBBP6_HUMAN | RBBP6 |
| sp\|Q8IY81\|SPB1_HUMAN | FTSJ3 | sp\|Q9H2F5\|EPC1_HUMAN | EPC1 | sp\|P55081\|MFAP1_HUMAN | MFAP1 |
| sp\|Q14526\|HIC1_HUMAN | HIC1 | sp\|Q5T310\|GPTC4_HUMAN | GPATCH4 | sp\|Q9UPT8\|ZC3H4_HUMAN | ZC3H4 |
| sp\|Q15233\|NONO_HUMAN | NONO | sp\|P13984\|T2FB_HUMAN | GTF2F2 | sp\|Q92576\|PHF3_HUMAN | PHF3 |
| sp\|Q14839\|CHD4_HUMAN | CHD4 | sp\|P18583\|SON_HUMAN | SON | sp\|Q9UHB7\|AFF4_HUMAN | AFF4 |
| sp\|Q13428\|TCOF_HUMAN | TCOF1 | sp\|P15822\|ZEP1_HUMAN | HIVEP1 | sp\|Q8WTT2\|NOC3L_HUMAN | NOC3L |
| sp\|P38432\|COIL_HUMAN | COIL | sp\|Q9Y2W2\|WBP11_HUMAN | WBP11 | sp\|Q9Y4C8\|RBM19_HUMAN | RBM19 |
| sp\|Q9UMN6\|KMT2B_HUMAN | KMT2B | sp\|Q15459\|SF3A1_HUMAN | SF3A1 | sp\|P49750\|YLPM1_HUMAN | YLPM1 |
| sp\|Q9UHR5\|S30BP_HUMAN | SAP30BP | sp\|Q76FK4\|NOL8_HUMAN | NOL8 | sp\|Q8WWY3\|PRP31_HUMAN | PRPF31 |
| sp\|P49321\|NASP_HUMAN | NASP | sp\|095696\|BRD1_HUMAN | BRD1 | sp\|075909\|CCNK_HUMAN | CCNK |
| sp\|Q9ULWO\|TPX2_HUMAN | TPX2 | sp\|Q8NI36\|WDR36_HUMAN | WDR36 | sp\|Q96T58\|MINT_HUMAN | SPEN |
| sp\|Q15652\|JHD2C_HUMAN | JMJD1C | sp\|O60216\|RAD21_HUMAN | RAD21 | sp\|Q96PZ0\|PUS7_HUMAN | PUS7 |
| sp\|Q03164\|KMT2A_HUMAN | KMT2A | sp\|O75663\|TIPRL_HUMAN | TIPRL | sp\|Q2VIQ3\|KIF4B_HUMAN | KIF4B |
| sp\|P61978\|HNRPK_HUMAN | HNRNPK | sp\|O60563\|CCNT1_HUMAN | CCNT1 | sp\|Q6IE81\|JADE1_HUMAN | JADE1 |
| sp\|Q9BTC0\|DIDO1_HUMAN | DIDO1 | sp\|P07737\|PROF1_HUMAN | PFN1 | sp\|Q6PJT7\|ZC3HE_HUMAN | ZC3H14 |
| sp\|Q8IX01\|SUGP2_HUMAN | SUGP2 | sp\|Q16630\|CPSF6_HUMAN | CPSF6 | sp\|Q8NI27\|THOC2_HUMAN | THOC2 |
| sp\|O43290\|SNUT1_HUMAN | SART1 | sp\|P49959\|MRE11_HUMAN | MRE11 | sp\|Q9Y2U8\|MAN1_HUMAN | LEMD3 |
| sp\|Q9H6Z4\|RANB3_HUMAN | RANBP3 | sp\|Q9UHX1\|PUF60_HUMAN | PUF60 | sp\|P21675\|TAF1_HUMAN | TAF1 |
| sp\|P09874\|PARP1_HUMAN | PARP1 | sp\|Q15637\|SF01_HUMAN | SF1 | sp\|Q9H267\|VP33B_HUMAN | VPS33B |
| sp\|Q92613\|JADE3_HUMAN | JADE3 | sp\|Q99459\|CDC5L_HUMAN | CDC5L | sp\|O75643\|U520_HUMAN | SNRNP200 |
| sp\|Q15020\|SART3_HUMAN | SART3 | sp\|Q13573\|SNW1_HUMAN | SNW1 | sp\|P22626\|ROA2_HUMAN | HNRNPA2B1 |
| sp\|Q96L91\|EP400_HUMAN | EP400 | sp\|Q9UK61\|TASOR_HUMAN | FAM208A | sp\|O60934\|NBN_HUMAN | NBN |
| sp\|Q13823\|NOG2_HUMAN | GNL2 | sp\|Q96KR1\|ZFR_HUMAN | ZFR | sp\|Q8N7H5\|PAF1_HUMAN | PAF1 |
| sp\|Q7Z5K2\|WAPL_HUMAN | WAPL | sp\|Q5T8P6\|RBM26_HUMAN | RBM26 | sp\|O75376\|NCOR1_HUMAN | NCOR1 |
| sp\|P46108\|CRK_HUMAN | CRK | sp\|043719\|HTSF1_HUMAN | HTATSF1 | sp\|Q13123\|RED_HUMAN | IK |
| sp\|P78347\|GTF2I_HUMAN | GTF2I | sp\|O95239\|KIF4A_HUMAN | KIF4A | sp\|Q14739\|LBR_HUMAN | LBR |
| sp\|Q8TDI0\|CHD5_HUMAN | CHD5 | sp\|Q14692\|BMS1_HUMAN | BMS1 | sp\|Q03188\|CENPC_HUMAN | CENPC |
| sp\|Q9Y2W1\|TR150_HUMAN | THRAP3 | sp\|O43143\|DHX15_HUMAN | DHX15 | sp\|Q9P013\|CWC15_HUMAN | CWC15 |
| sp\|Q12873\|CHD3_HUMAN | CHD3 | sp\|Q92541\|RTF1_HUMAN | RTF1 | sp\|Q13330\|MTA1_HUMAN | MTA1 |
| sp\|O75533\|SF3B1_HUMAN | SF3B1 | sp\|Q14966\|ZN638_HUMAN | ZNF638 | sp\|P53814\|SMTN_HUMAN | SMTN |
| sp\|P55201\|BRPF1_HUMAN | BRPF1 | sp\|O14776\|TCRG1_HUMAN | TCERG1 | sp\|P52597\|HNRPF_HUMAN | HNRNPF |
| sp\|P53680\|AP2S1_HUMAN | AP2S1 | sp\|Q14241\|ELOA1_HUMAN | ELOA | sp\|Q9BWUO\|NADAP_HUMAN | SLC4A1AP |
| sp\|O60885\|BRD4_HUMAN | BRD4 | sp\|P29590\|PML_HUMAN | PML | sp\|Q7Z5J4\|RAI1_HUMAN | RAI1 |
| sp\|P42166\|LAP2A_HUMAN | TMPO | sp\|Q9BRR8\|GPTC1_HUMAN | GPATCH1 | sp\|Q6NZY4\|ZCHC8_HUMAN | ZCCHC8 |
| sp\|Q5VUA4\|ZN318_HUMAN | ZNF318 | sp\|P23246\|SFPQ_HUMAN | SFPQ | sp\|Q3L8U1\|CHD9_HUMAN | CHD9 |
| sp\|Q52LR7\|EPC2_HUMAN | EPC2 | sp\|Q9H1E3\|NUCKS_HUMAN | NUCKS1 | sp\|Q92917\|GPKOW_HUMAN | GPKOW |
| sp\|O00763\|ACACB_HUMAN | ACACB | sp\|Q96T23\|RSF1_HUMAN | RSF1 | sp\|P28340\|DPOD1_HUMAN | POLD1 |
| sp\|O15042\|SR140_HUMAN | U2SURP | sp\|Q5QJE6\|TDIF2_HUMAN | DNTTIP2 | sp\|Q69YH5\|CDCA2_HUMAN | CDCA2 |
| sp\|Q9ULM3\|YETS2_HUMAN | YEATS2 | sp\|QUIPO\|RIF1_HUMAN | RIF1 | sp\|Q8N163\|CCAR2_HUMAN | CCAR2 |
| sp\|Q13263\|TIF1B_HUMAN | TRIM28 | sp\|Q14814\|MEF2D_HUMAN | MEF2D | sp\|P43243\|MATR3_HUMAN | MATR3 |
| sp\|Q86VM9\|ZCH18_HUMAN | ZC3H18 | sp\|Q14865\|ARI5B_HUMAN | ARID5B | sp\|Q86YC2\|PALB2_HUMAN | PALB2 |
| sp\|Q1ED39\|KNOP1_HUMAN | KNOP1 | sp\|Q92794\|KAT6A_HUMAN | KAT6A | sp\|O00566\|MPP10_HUMAN | MPHOSPH10 |
| sp\|P51003\|PAPOA_HUMAN | PAPOLA | sp\|Q9H412\|ZHX3_HUMAN | ZHX3 | sp\|Q96QC0\|PP1RA_HUMAN | PPP1R10 |
| sp\|Q8WYP5\|ELYS_HUMAN | AHCTF1 | sp\|Q92945\|FUBP2_HUMAN | KHSRP | sp\|Q9Y4E5\|ZN451_HUMAN | ZNF451 |
| sp\|Q9H5V9\|CX056_HUMAN | CXorf56 | sp\|Q02241\|KIF23_HUMAN | KIF23 | sp\|Q8NFC6\|BD1L1_HUMAN | BODIL1 |
| sp\|Q9H0AO\|NAT10_HUMAN | NAT10 | sp\|O00567\|NOP56_HUMAN | NOP56 | sp\|095391\|SLU7_HUMAN | SLU7 |
| sp\|P55265\|DSRAD_HUMAN | ADAR | sp\|Q7Z4V5\|HDGR2_HUMAN | HDGFL2 | sp\|O15381\|NVL_HUMAN | NVL |
| sp\|P98175\|RBM10_HUMAN | RBM10 | sp\|Q96PV6\|LENG8_HUMAN | LENG8 | sp\|A3KN83\|SBNO1_HUMAN | SBNO1 |
| sp\|Q08211\|DHX9_HUMAN | DHX9 | sp\|Q9HCS7\|SYF1_HUMAN | XAB2 | sp\|Q9NQC1\|JADE2_HUMAN | JADE2 |
| sp\|Q9NYF8\|BCLF1_HUMAN | BCLAF1 | sp\|Q12789\|TF3C1_HUMAN | GTF3C1 | sp\|094913\|PCF11_HUMAN | PCF11 |
| sp\|Q9HCK8\|CHD8_HUMAN | CHD8 | sp\|Q9BVJ6\|UT14A_HUMAN | UTP14A | sp\|Q9UBC2\|EP15R_HUMAN | EPS15L1 |
| sp\|Q86XP3\|DDX42_HUMAN | DDX42 | sp\|Q12888\|TP53B_HUMAN | TP53BP1 | sp\|P33991\|MCM4_HUMAN | MCM4 |
| sp\|Q9BQG0\|MBB1A_HUMAN | MYBBP1A | sp\|Q9UBB9\|TFP11_HUMAN | TFIP11 | sp\|P46109\|CRKL_HUMAN | CRKL |
| sp\|Q8IWZ8\|SUGP1_HUMAN | SUGP1 | sp\|Q9Y2X3\|NOP58_HUMAN | NOP58 | sp\|Q8TEK3\|DOT1L_HUMAN | DOT1L |
| sp\|Q9H501\|ESF1_HUMAN | ESF1 | sp\|Q15061\|WDR43_HUMAN | WDR43 | sp\|Q6PL18\|ATAD2_HUMAN | ATAD2 |
| sp\|Q6IN85\|P4R3A_HUMAN | PPP4R3A | sp\|P35637\|FUS_HUMAN | FUS | sp\|Q96JK2\|DCAF5_HUMAN | DCAF5 |
| sp\|O75152\|ZC11A_HUMAN | ZC3H11A | sp\|Q96RE7\|NACC1_HUMAN | NACC1 | sp\|Q13129\|RLF_HUMAN | RLF |
| sp\|Q03701\|CEBPZ_HUMAN | CEBPZ | sp\|O75717\|WDHD1_HUMAN | WDHD1 | sp\|Q2NL82\|TSR1_HUMAN | TSR1 |
| sp\|P46100\|ATRX_HUMAN | ATRX | sp\|Q15648\|MED1_HUMAN | MED1 | sp\|Q14684\|RRP1B_HUMAN | RRP1B |
| sp\|P17480\|UBF1_HUMAN | UBTF | sp\|Q9NQZ2\|SAS10_HUMAN | UTP3 | sp\|O75150\|BRE1B_HUMAN | RNF40 |
| sp\|Q9NR30\|DDX21_HUMAN | DDX21 | sp\|Q9NXR8\|ING3_HUMAN | ING3 | sp\|P28715\|ERCC5_HUMAN | ERCC5 |
| sp\|Q9P2N5\|RBM27_HUMAN | RBM27 | sp\|000193\|SMAP_HUMAN | SMAP | sp\|Q9BSC4\|NOL10_HUMAN | NOL10 |
| sp\|P17844\|DDX5_HUMAN | DDX5 | sp\|Q8WUQ7\|CATIN_HUMAN | CACTIN | sp\|Q14687\|GSE1_HUMAN | GSE1 |
| sp\|Q96KK5\|H2A1H_HUMAN | HIST1H2AH | sp\|Q5JTH9\|RRP12_HUMAN | RRP12 | sp\|Q03111\|ENL_HUMAN | MLLT1 |
| sp\|Q01780\|EXOSX_HUMAN | EXOSC10 | sp\|Q8IWX8\|CHERP_HUMAN | CHERP | sp\|Q92620\|PRP16_HUMAN | DHX38 |
| sp\|Q96EV2\|RBM33_HUMAN | RBM33 | sp\|Q15059\|BRD3_HUMAN | BRD3 | sp\|P51587\|BRCA2_HUMAN | BRCA2 |
| sp\|Q7LBC6\|KDM3B_HUMAN | KDM3B | sp\|O00148\|DX39A_HUMAN | DDX39A | sp\|P05455\|LA_HUMAN | SSB |
| sp\|Q96ST3\|SIN3A_HUMAN | SIN3A | sp\|P49790\|NU153_HUMAN | NUP153 | sp\|Q6P2Q9\|PRP8_HUMAN | PRPF8 |
| sp\|O43395\|PRPF3_HUMAN | PRPF3 | sp\|P46087\|NOP2_HUMAN | NOP2 | sp\|P26599\|PTBP1_HUMAN | PTBP1 |
| sp\|Q5VT52\|RPRD2_HUMAN | RPRD2 | sp\|Q14690\|RRP5_HUMAN | PDCD11 | sp\|P12956\|XRCC6_HUMAN | XRCC6 |

To evaluate for changes in the MEAF6 proximal proteome as a consequence of Menin inhibition (alone or in combination with MOZ inhibition), MEAF6-BirA expressing GIST-T1 cells were pre-treated for 3 days with VTP-50469 alone or in combination with WM-1119 prior to labeling with biotin and subsequent label-free quantification using mass spectrometry. While the majority of MEAF6-proximal proteins remained the same, a subset of proteins showed significant alterations in abundance with drug treatment, with significant correlation in changes seen with VTP-50469 and WM-1119 (FIG. 6D). MLL family members KMT2A/MLL1, KMT2B/MLL2 and DNA and RNA binding anti-apoptotic protein GPATCH4 (Lambert et al., J. Proteomics 118:81-94 (2015)) interactions were decreased with VTP-50469 treatment alone, combinational treatment with VTP-50469 and WM-1119 had no additional effect (FIG. 6F). Proximity of select chromatin regulators, splicing factors and polymerase regulatory proteins were altered in a similar fashion with VTP-50469 alone or in combination with WM-1119, most notably DOT1L which was significantly decreased in all treatment conditions (FIG. 6G).

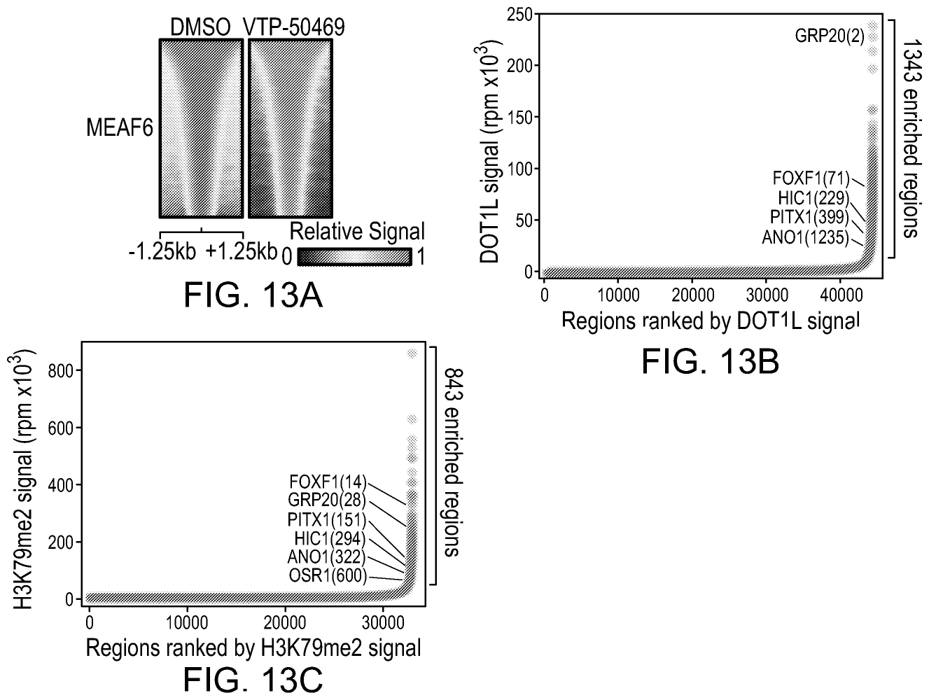

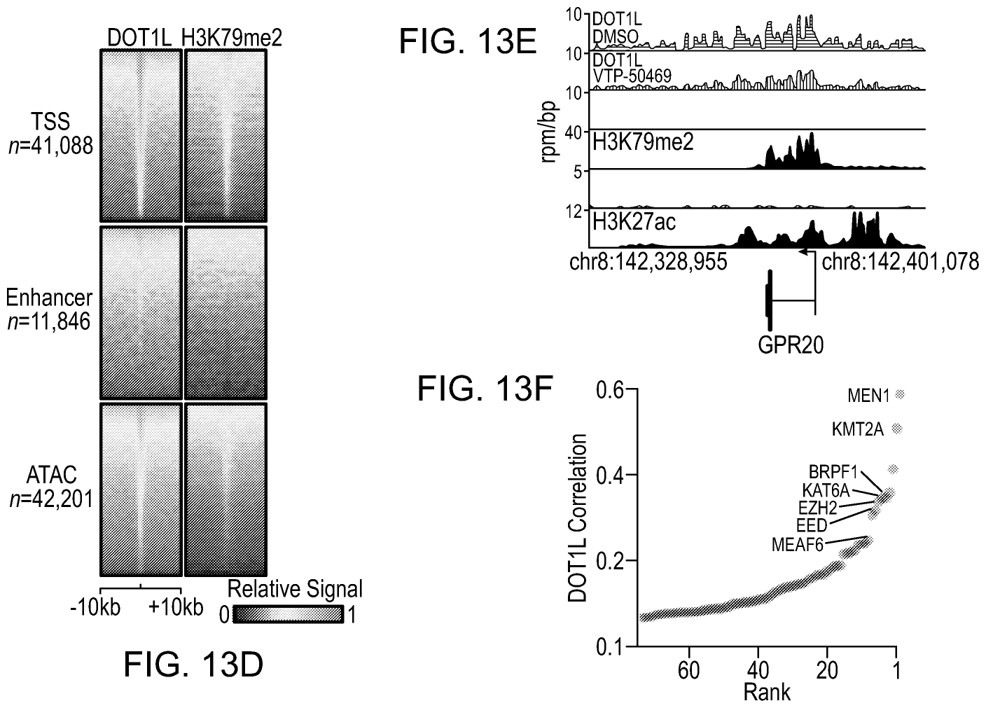

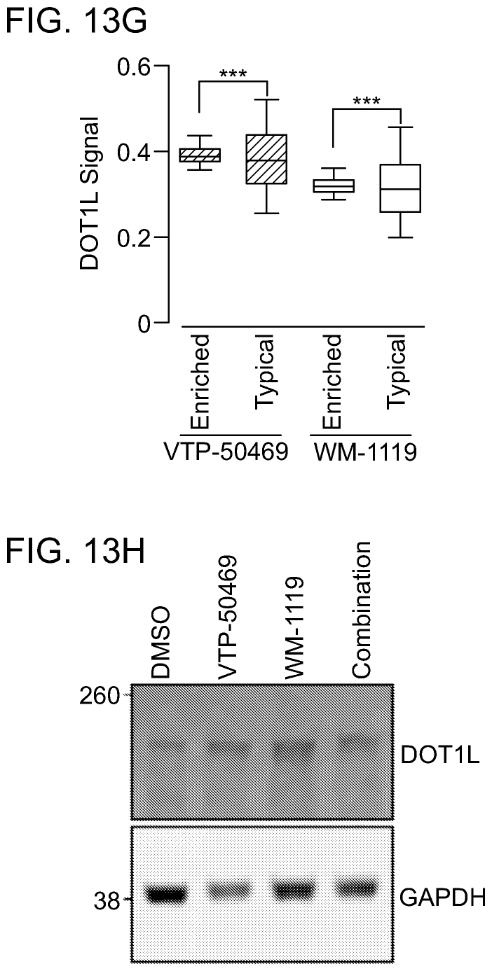

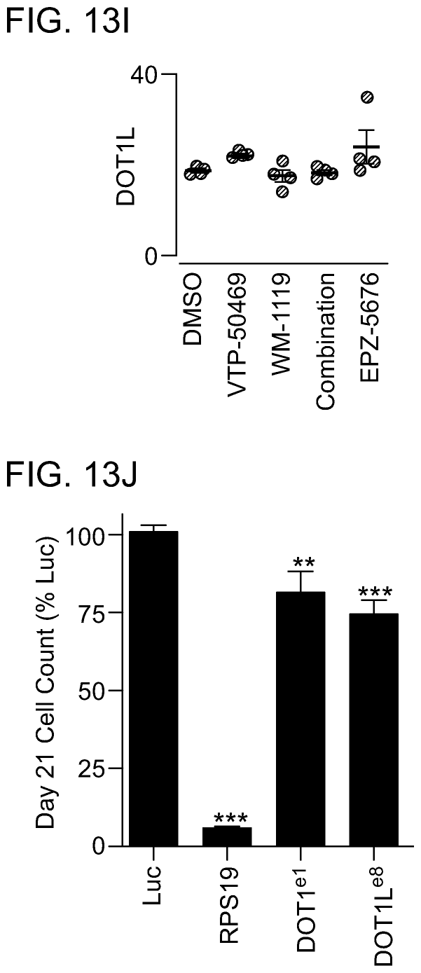

To determine how Menin inhibition altered chromatin association of DOT1L, spike-in normalized ChIP-seq in GIST-T1 cells were treated with VTP-50469 alone or in combination, which significantly decreased DOT1L association with chromatin at all DOT1L binding sites, with reductions in average DOT1L signal genome-wide, as illustrated in FIGS. 6G-6I and FIG. 13G-FIG. 13I. FIG. 6G has heat maps demonstrating spike-in normalized signal of DOT1L at MACS-defined peaks (n=67,769) in GIST-T1 cells treated with DMSO, VTP-50469 for 3 days. Scaled read densities+1.25 kb from the peak center are shown in rows. FIGS. 6I-6J have an n of 67.769 for DOT1L signal and an n of 22.581 for MEAF6 signal. P<0.001: **. P<0.01: *. P<0.05.

Like other Menin-MLL complex members. DOT1L. H3K79me2. the histone mark deposited by DOT1L, and MEAF6 all showed enrichment genome wide at the TSS and gene body of active genes, with enrichment at loci relevant to GIST biology and reduction in DOT1L signal with VTP-50469 treatment, as illustrated in FIGS. 6K and 13B-13E. FIG. 13D shows heat maps demonstrating genomic localization in GIST-T1 of DOT1L, and H3K79me2 by ChIP-seq. Scaled read densities±10 kb from the TSS. H3K27ac-defined super enhancers or ATAC-defined peaks are shown in rows. FIG. 13E includes tracks showing regions of genomic occupancy of spike-in normalized DOT1L under the indicated treatments. H3K79me2, and H3K27ac at the GPR20 locus. DOT1L TE vs SE. SE=1343. TE=45256. Welch's T-test P<0.001. but absolute difference ˜1%. To further explore whether loss of DOT1L function could constitute a mechanism of cellular toxicity downstream of Menin inhibition. GIST-T1 cells or GIST48B as control, were treated in a growth over time assay at various doses of the selective DOT1L inhibitor EPZ-5676 (Daigle et al., Cancer Cell 20:53-65 (2011)). At all doses tested. GIST-T1 exhibited significantly reduced cellular proliferation compared to DMSO control or GIST48B (with 5 per condition). indicating selective toxicity of DOT1L inhibition similar to Menin inhibition, as illustrated in FIG. 6L.

DOT1L-targeting sgRNAs led to significant reductions in GIST-T1 cell proliferation, although more modest than that seen with Menin-MLL and MOZ complex-targeting sgRNAs, consistent with the findings of the genome-scale CRISPR screen (FIG. 2A and FIG. 13I). To better characterize the transcriptional consequences of DOT1L inhibition in GIST. GIST-T1 cells were treated with EPZ-5676 for 5 days followed by RNA-seq. Globally. transcriptional changes associated with EPZ-5676 treatment were highly correlated with those arising from Menin inhibition with VTP-50469 (FIG. 6M-FIG. 6N), a phenomenon previously observed in MLL-rearranged leukemia. Like Menin-MLL and MOZ inhibition, DOT1L inhibition led to significant disruption in HAND1-regulated transcriptional programs (FIG. 6O), with alterations in the expression of KIT. CD34. NPR3, and GIST TFs (FIG. 6P-FIG. 6Q). Together, these data demonstrate the complexity of proximal protein interactions between these epigenetic complexes, alterations in the protein and chromatin associations of multiple transcriptional regulators with MOZ or Menin inhibition, and that DOT1L function is a dependency in KIT-dependent GIST, with loss of DOT1L chromatin association serving as a downstream consequence of MOZ or Menin inhibition. Above, growth over time experiments were analyzed by two-way ANOVA with Tukey's post-hoc test, compared to GIST48B: ***P<0.001: *. P<0.05. Together, these data demonstrate the complexity of proximal protein interactions between these epigenetic complexes, alterations in the protein and chromatin associations of multiple transcriptional regulators with Menin inhibition, and that DOT1L function is a dependency in KIT-dependent GIST, with loss of DOT1L chromatin association serving as a downstream consequence of Menin inhibition.

### Example 6: Therapeutic Effects of Menin Inhibition In Vivo Alone or in Combination with TKI

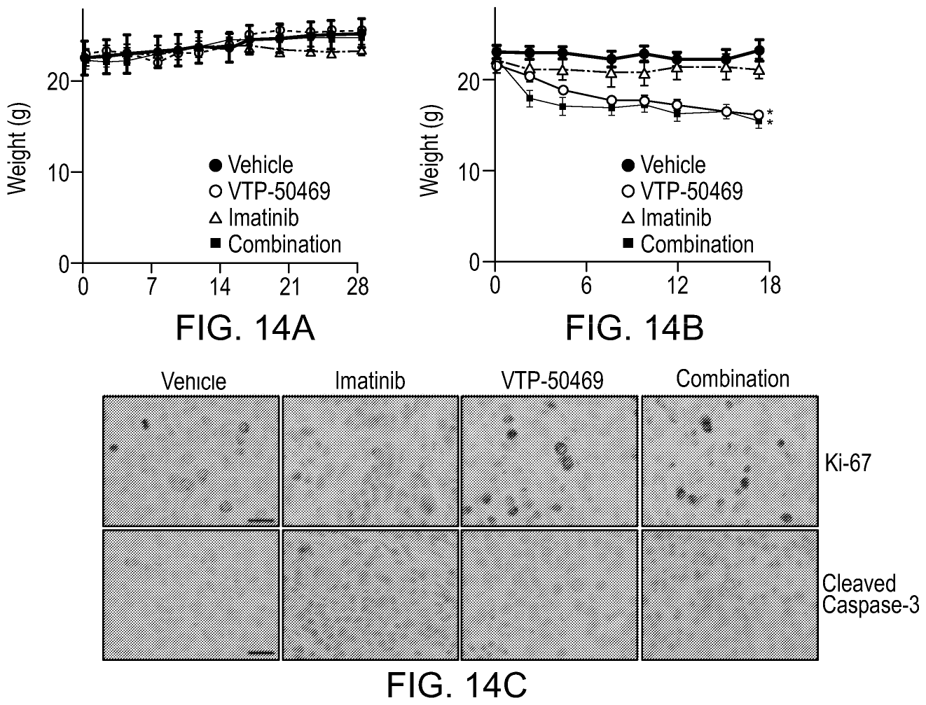

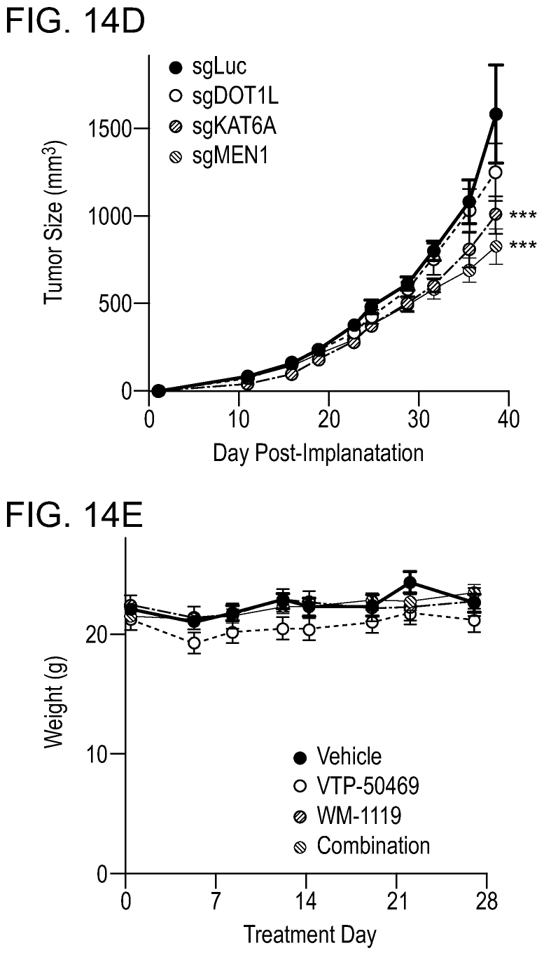

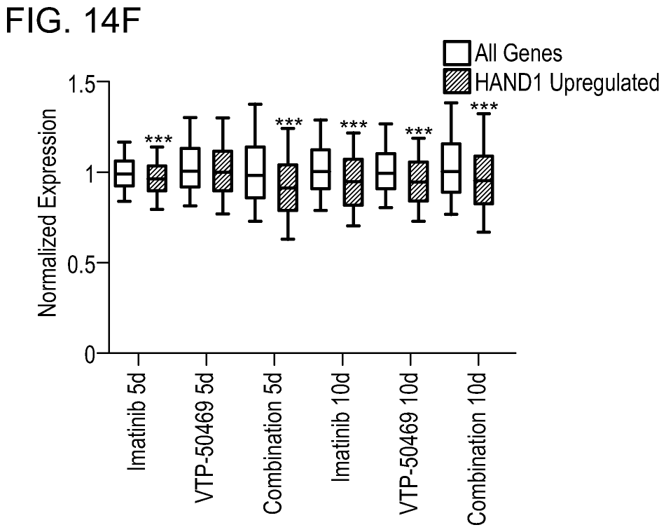

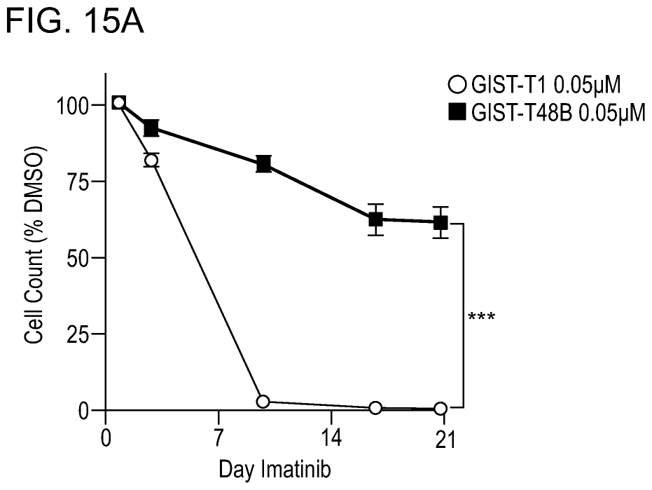

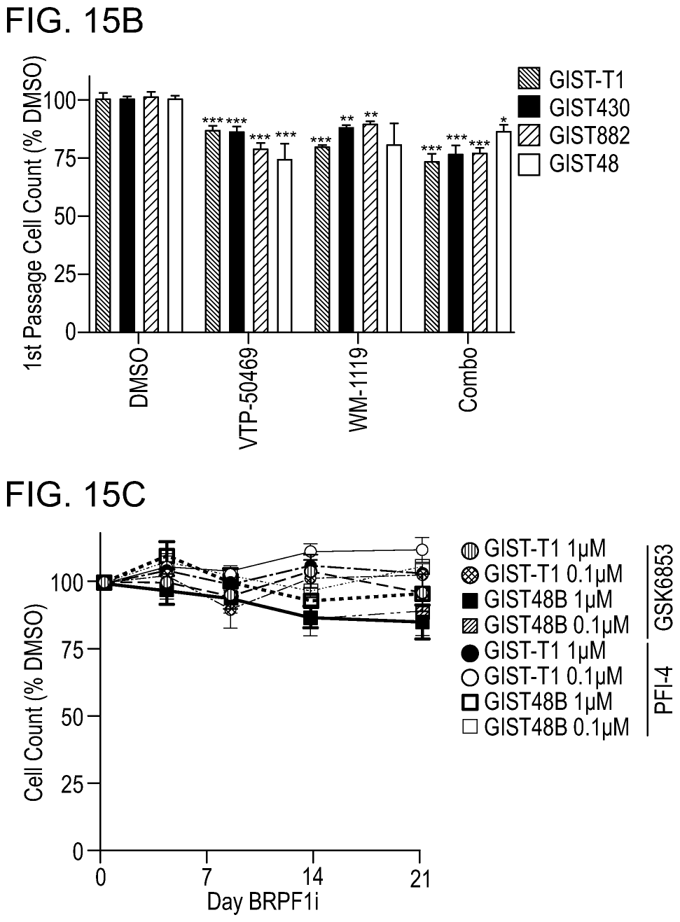

To evaluate the effects of genetic loss of KAT6A, Menin, or DOT1L on tumor growth in vivo, cells expressing an sgRNA directed against each of KAT6A (sgKAT6A), Menin (sgMEN1), or DOT1L (sgDOT1L) or luciferase control (sgLuc) in GIST-T1 cells co-expressing Cas9 were prepared. Following implantation of an equal number of modified cells, mice were monitored for tumor formation and growth. Although all implants generated tumors, those derived from cells treated with sgKAT6A or sgMEN1 had significantly reduced growth compared with sgLuc control, whereas expression of sgDOT1L led to a nonsignificant trend toward reduced growth (FIG. 14D). Although growth restriction was more modest than analogous in vitro experiments (FIG. 2H and FIG. 13I), sgKAT6A and sgMEN1 conditions required selection and propagation for 2 weeks in vitro to produce enough cells for implantation, likely selecting for cells with less deleterious gene alterations.

Multiple Menin inhibitors have advanced to early phase clinical trials for the treatment of leukemia, including National Clinical Trials (NCT) numbers NCT04067336 (a study of compound KO539), NCT04811560 (a study of compound JNJ-75276617), and NCT04065399 (a study of compound SNDX-5613). KO539 is also known as Unii-4mod 1F4enc and ziftomenib.

To assess the effects of Menin inhibition alone or in combination with WM-1119 or imatinib in vivo, mice were engrafted with GIST-T1 cells and treated with imatinib (n=5), VTP-50469 (given continuously in chow: n=4), WM-1119 (dosed 3 times daily 7 days/week n=6), the combination of VTP-50469 and WM-1119 (n=6), the combination of imatinib and VTP-50469 (n=5), or vehicle control (n=5). At the end of a 28-day treatment period, the monotherapy treatment groups showed similar significant reductions in tumor growth compared to vehicle, while the combination groups showed complete cessation of tumor growth. Tumor recovery monitoring was continued without further drug treatment, and while tumors from imatinib and VTP-50469 monotherapy groups regained a tumor growth trajectory similar to the vehicle group, the combination of imatinib and VTP-50469 sustained a 3-4-fold reduced slope of tumor recovery, as illustrated in FIG. 7A. Tumor volume relative to baseline is detailed in Table 5 for vehicle control, Table 6 for VTP-50469, and Table 7 for WM-1119, Table 8 for the combination of VTP-50469 and VM-1119. During the continued monitoring of tumor recovery without further drug treatment all conditions showed comparable tumor growth rates similar to vehicle control. The combination therapy of VTP-50469 and WM-1119 had a similar tumor growth trajectory as the imatinib and VTP-50469 therapy, as illustrated in FIG. 7D. Mice tolerated treatment with WM-1119, VTP-50469, imatinib, and a combination thereof without evidence of overt toxicity or weight loss (FIG. 14E-FIG. 14F).

TABLE 5
Vehicle control tumor volume relative to baseline

| Treatment | Animal | | | | |
|---|---|---|---|---|---|
| Day | 1 | 2 | 3 | 4 | 5 |
| 1 | 100 | 100 | 100 | 100 | 100 |
| 5 | 167 | 184 | 239 | 171 | 243 |
| 8 | 177 | 325 | 426 | 193 | 335 |
| 12 | 213 | 389 | 502 | 225 | 379 |
| 14 | 259 | 405 | 546 | 253 | 510 |
| 19 | 265 | 430 | 683 | 255 | 664 |
| 22 | 317 | 438 | 713 | 487 | 689 |
| 27 | 368 | 455 | 948 | 593 | 759 |
| 29 | 384 | 522 | 967 | 578 | 876 |

TABLE 6
VTP-50469 tumor volume relative to baseline

| | |
|---|---|
| Treatment | Animal |

| Day | 1 | 2 | 3 | 4 | 5 | 6 |
|---|---|---|---|---|---|---|
| 1 | 100 | 100 | 100 | 100 | 100 | 100 |
| 5 | 191 | 172 | 102 | 182 | 68 | 160 |
| 8 | 201 | 188 | 107 | 178 | 67 | 284 |
| 12 | 221 | 290 | 90 | 176 | 59 | 486 |
| 14 | 265 | 356 | 136 | 276 | 73 | 519 |
| 19 | 383 | 399 | 188 | 379 | 43 | 832 |
| 22 | 353 | 440 | 128 | 480 | 71 | 1108 |
| 27 | 622 | 466 | 119 | 735 | 80 | 1469 |
| 29 | 773 | 461 | 153 | 962 | 92 | * |

mouse terminated early due to rapid tumor growth

TABLE 7
WM-1119 tumor volume relative to baseline

| | |
|---|---|
| Treatment | Animal |

| Day | 1 | 2 | 3 | 4 | 5 | 6 |
|---|---|---|---|---|---|---|
| 1 | 100 | 100 | 100 | 100 | 100 | 100 |
| 5 | 175 | 211 | 114 | 133 | 87 | 152 |
| 8 | 480 | 322 | 157 | 173 | 131 | 206 |
| 12 | 518 | 368 | 287 | 160 | 145 | 260 |
| 14 | 527 | 429 | 327 | 181 | 163 | 328 |
| 19 | 876 | 501 | 511 | 180 | 268 | 277 |
| 22 | 878 | 409 | 489 | 150 | 205 | 361 |
| 27 | 856 | 525 | 558 | 202 | 232 | 317 |
| 29 | 874 | 532 | 648 | 199 | 221 | 353 |

TABLE 8
Combination VTP-50469 and WM-1119
tumor volume relative to baseline

| | |
|---|---|
| Treatment | Animal |

| Day | 1 | 2 | 3 | 4 | 5 | 6 |
|---|---|---|---|---|---|---|
| 1 | 100 | 100 | 100 | 100 | 100 | 100 |
| 5 | 100 | 128 | 79 | 242 | 159 | 103 |
| 8 | 119 | 138 | 90 | 288 | 138 | 115 |
| 12 | 98 | 111 | 66 | 384 | 104 | 92 |
| 14 | 71 | 104 | 64 | 316 | 114 | 121 |
| 19 | 79 | 123 | 45 | 445 | 45 | 98 |
| 22 | 61 | 155 | 53 | 493 | 49 | 112 |
| 27 | 32 | 159 | 43 | 518 | 46 | 101 |
| 29 | 67 | 198 | 46 | 613 | 57 | 98 |

RNA-seq on GIST-T1 xenografts after 5 and 10 days of imatinib and/or VTP-50469 treatment was performed to evaluate for changes in the GIST transcriptional program arising from Menin and/or KIT inhibition in vivo. Although all treatment conditions led to global transcriptional changes compared with vehicle control, greater changes were seen following treatment with VTP-50469 and the combination of imatinib and VTP-50469 at both time points, with the gene expression profile of imatinib treatment more closely correlating with vehicle-treated tumors (FIG. 7E). FIG. 7E shows the Pearson correlation of group-averaged fragments per kilobase of transcript per million mapped reads (FPKM) of all expressed genes (FPKM>10, n=7,434). Imatinib, VTP-50469, and combination treatments all led to a decrease in the expression of genes regulated by HAND1 (FIG. 14G). Although GIST-associated transcripts including KIT, CD34, and NPR3 were preferentially reduced by VTP-50469 treatment, other KIT signaling-dependent transcripts including TMEM100 and SPRY2 were preferentially reduced with imatinib treatment. PCNA, a marker of cellular proliferation, was reduced only with the combination of imatinib and VTP-50469 at both 5- and 10-day time points (FIG. 7F), consistent with the greater effect of the combination treatment on tumor growth.

Next the effects of imatinib and VTP-50469 treatment on PG27, a KIT mutant patient derived xenograft (PDX) model of GIST (Hemming et al., Cancer Res. 79 (5): 994-1009 (2019)) was evaluated. While imatinib administration below the maximal tolerated dose had a significant but modest growth inhibitory effect compared to the GIST-T1 cell line xenograft, treatment with VTP-50469 alone (n=5) or in combination with imatinib (n=5) resulted in a significant reduction in tumor growth, as illustrated in FIG. 7B. Data were analyzed by two-way ANOVA, compared to vehicle: ***, P<0.001: compared to imatinib: #, P<0.01. Whereas mice treated with VTP-50469 in chow (n=5) showed no weight loss in the GIST-T1 xenograft experiment, PG27 mice treated with a different batch of VTP-50469 at the same concentration exhibited modest weight loss (see, FIGS. 14A-14B, data analyzed by two-way ANOVA, compared to vehicle: *, P<0.05). At the end of the treatment period. PG27 tumors were harvested, fixed, and sectioned to evaluate tumor histology. While vehicle and imatinib treated tumors had monomorphic sheets of tumor cells, xenografts treated with VTP-50469 or drug combination exhibited areas of tumor necrosis, as illustrated in FIG. 7C. Representative images in FIG. 7C are shown from treatment groups at 4× magnification (upper panels, scale bar=250) μm) and 40× magnification (lower panels, scale bar=25 μm). Despite restriction of tumor growth with VTP-50469 treatment, viable areas of tumor showed similar levels of Ki-67 and cleaved caspase-3 across conditions (see, FIG. 14C, where PG27 tumors were harvested at the end of the treatment period and fixed tissues sectioned and evaluated for Ki-67 (top row) and cleaved caspase-3 (bottom row): scale bar=25 μm). Whereas mice treated with VTP-50469 in chow showed no weight loss in the GIST-T1 xenograft experiments, PG27-engrafted mice treated with VTP-50469 at the same concentration exhibited modest weight loss (FIG. 14B), possibly related to systemic effects of the observed tumor necrosis. Despite the restriction of tumor growth with VTP-50469 treatment, viable areas of the tumor showed similar levels of Ki-67 and cleaved caspase-3 across conditions (FIG. 14B). These data demonstrate the therapeutic activity of VTP-50469 alone or in combination with imatinib, that Menin inhibition decreases GIST xenograft growth, produces tumor necrosis and, when combined with imatinib, generates durable anti-tumor responses after cessation of treatment.

These embodiments show that the organization and remodeling of chromatin is essential to cellular lineage, identity and function. Post-translational modifications of histones serve as a nexus of epigenetic regulation that controls binding of TFs and chromatin regulators, ultimately administrating gene expression and chromosomal structure. Chromatin modifications are dynamic and reversible, they require active maintenance by cell type and state specific chromatin modifying enzymes. Cancer exploits or appropriates the chromatin state of its precursor cells to sustain a malignant phenotype, through maintenance of an environment permissive of oncogene activation or by gain-of-function alterations in chromatin regulators such as MLL gene fusions (Krivtsov et al., Nat. Rev. Cancer 7:823-33 (2007)). Here, the present disclosure shows that specific chromatin regulators are essential to sustain the GIST epigenome, with the Menin-MLL complex binding to actively expressed genes genome-wide, regulating GIST-associated gene expression programs, coordinating protein-protein interactions between multiple regulators of gene expression, and ultimately regulating cellular proliferation and tumor growth.

Menin, encoded by the MEN1 gene, has classically been described as a tumor suppressor, with mutations in MEN1 promoting endocrine tumor formation. However, in other tissues multiple functions have been ascribed to Menin arising from the protein's ability to positively or negatively regulate gene expression, associate with different chromatin complexes, integrate inputs from upstream signaling pathways and modulate DNA replication and repair (Matkar et al., Trends Biochem. Sci. 38 (8): 394-402 (2013)). Menin has been best studied as an oncogenic dependency in the context of MLL-rearranged leukemia, where it binds to the MLL fusion protein and, together with recruitment of DOT1L, executes the leukemogenic gene expression program (Krivtsov et al., Cancer Cell 36:660-673 (2019). Yokoyama et al., Cell 123:207-18 (2005), Dafflon et al., Leukemia 31:1269-77 (2017)). In GIST, Menin-MLL complex members are essential for global chromatin regulation and, ultimately, tumor cell proliferation. Compared to hundreds of other cell types profiled in Project DRIVE and DepMap. GIST has exceptional sensitivity to targeted disruption of Menin-MLL complex members. In agreement with the conservation of TFs and transcriptional and chromatin landscapes in KIT-dependent GIST (Hemming et al., Proc. Natl. Acad. Sci. USA 115:E5746-55 (2018), Dafflon et al., Leukemia 31:1269-77 (2017)), sensitivity to genetic or pharmacologic Menin-MLL complex disruption was lost in a KIT-independent GIST cell line. These data indicate that, unlike the oncogenic hijacking seen in MLL-rearranged leukemias, GIST depends upon the native function of the Menin-MLL complex and their associated dependencies to maintain a chromatin landscape that provides a foundation for a malignant gene expression program.

Multiple lines of evidence suggest collaboration among Menin-MLL, MOZ, and other complexes in transcriptional regulation. Here, this disclosure shows genome-wide colocalization of Menin-MLL and MOZ complex members at the TSS of actively expressed genes, similar changes in gene expression arising from inhibition of either complex, proximal protein interactions between these two complexes, coordinated regulation of DOT1L and other transcription-associated proteins, and that effects on cell cycle and cellular proliferation were more marked when inhibiting the Menin-MLL complex in combination with MOZ complex inhibition. In agreement with the findings disclosed herein, interactions between MOZ and MLL complexes promoting gene expression have been previously described at the HOXA locus in hematopoietic progenitor cells (Paggetti et al., Oncogene 29:5019-31 (2010)). Leveraging DepMap data, the present disclosure highlights a previously underappreciated and complementary genetic co-dependency of these chromatin regulatory complexes in a minority of cancer cell lines. Dependency upon the PRC2 complex was also seen in GIST, with similar co-dependency observed across DepMap data, suggesting the contrasting but complementary role of PRC2 in chromatin silencing balancing the activating functions of the Menin-MLL complex.

These data also suggest that Menin-MLL and PRC2 complexes cooperatively function genome-wide to control chromatin state and transcriptional output. Though this disclosure highlights superior activity of simultaneous inhibition of Menin-MLL and MOZ complexes with VTP-50469 alone or in combination with WM-1119 on cell cycle and cellular proliferation assays, expression of select GIST-associated genes and disruption in protein-protein interactions was largely similar between monotherapy and combination treatments. While the mechanism of combinatorial toxicity requires further investigation, these results suggest that disruption of one complex may maximally deregulate both at specific target loci. and that non-overlapping functions of Menin-MLL and MOZ complexes are likely to exist.

In keeping with its association with the TSSs of active genes genome-wide, disruption of the Menin-MLL complex led to broad alterations in transcription that were both modest and enriched in specific pathways. Genes bearing the greatest enrichment of these chromatin complexes had significantly reduced gene expression with Menin inhibition. This disclosure also shows disproportionate reductions in transcription of genes essential to GIST. as well as in genes downregulated by imatinib treatment, suggesting the foundational role that the Menin-MLL complex plays in supporting transcription downstream of KIT signaling. Using GSEA, transcriptional changes arising from Menin inhibition were significantly associated with gene sets indicating reduced cell cycle and mitogenic signaling, and activation of developmental and EMT programs. Previous studies have observed upregulation of an EMT signature in less aggressive forms of GIST or following disruption of the oncogenic TF HAND1 (Hemming et al., Clin. Cancer Res. 27:1706-19 (2021)), indicating convergence of transcriptional pathways with either TF disruption or pharmacologic chromatin regulator inhibition. In keeping with Menin-MLL disruption causing modest changes in gene expression, effects on proliferation and cell cycle were only observed after several days of drug treatment. in contrast to the acutely toxic effects of imatinib.

Downstream consequences of Menin inhibition include disruption of proximal interactions between the multiple transcriptional regulators disclosed herein, including loss of DOT1L from chromatin. DOT1L methylates H3K79 to support an active transcriptional state, and has been investigated in leukemia where its recruitment by the MLL fusion protein is essential for leukemogenesis (Okada et al., Cell. 121:167-78 (2005)). In solid tumors, DOT1L has been found to cooperate with oncogenic transcription factors (Wong et al., Cancer Research 77:2522-33 (2017), Vatapalli et al., Nat. Commun. 11 (1): 4153 (2020)), though DOT1L inhibitors have thus far not been evaluated in clinical trial for solid tumors. With prior studies demonstrating TF dependencies in GIST, and current work showing the vulnerability of GIST cells to both DOT1L genetic and pharmacologic disruption, DOT1L may function as a downstream integrator of TF and Menin-MLL complex activity in establishing a transcriptionally active state of select cancer-associated genes.

Taken together, these data demonstrate the essential function of the Menin-MLL complex in GIST, which serves as an integral component of chromatin regulation and the oncogenic gene expression program.

Multiple Menin inhibitors have been developed that disrupt the association between Menin and MLL (Krivtsov et al., Cancer Cell. 36 (6): 660-673 (2019), Klossowski et al., J. Clin. Invest. 130:981-97 (2020), Xu et al., J. Med. Chem. 63:4997-5010 (2020)) and are now under clinical investigation for leukemia. To assess the in vivo effect of Menin inhibition on xenograft models of GIST, this disclosure describes treatment of cell line and patient-derived xenografts with TKI, Menin inhibition, or a combination treatment, which demonstrated activity of Menin inhibition as a monotherapy and even greater activity with the combination of TKI and Menin inhibition. After the treatment period, tumors in both monotherapy arms regained their growth trajectory, while tumors treated with the Menin inhibition and TKI combination therapy sustained prolonged tumor suppressive effects observed weeks after treatment withdrawal. In the above disclosures a PDX model of GIST saw potent anti-tumor activity of Menin inhibition, with histology showing areas of necrosis interspersed with viable tumor. These results support the clinical development of Menin inhibitors for GIST patients, either alone or ideally in combination with TKIs.

As TKIs are the only active therapeutic strategy in GIST, which has native resistance to cytotoxic chemotherapy (Maki et al., Oncologist 20 (7): 823-30 (2015)), targeting Menin and other essential components of the GIST epigenome may prove therapeutically advantageous. The conserved transcriptional and enhancer landscape seen in GIST tumors and cell lines, together with oncogenic KIT gene expression being regulated by disease-specific TF and enhancer elements, harbingers this disease's dependency upon epigenetic mechanisms of disease regulation. As described herein, the collaborating chromatin regulators responsible for maintaining the GIST epigenome, and how their disruption at multiple disparate nodes with small molecule inhibitors (e.g., VTP-50469, EPZ-5676) displays promising and selective anti-cancer activity: members of each of these inhibitor classes have reached clinical trial (e.g., NCT04606446, NCT02141828). As compared with leukemias bearing oncogenic alterations in chromatin regulators, GIST may be an outlier among solid tumors in its dependency upon these pathways and susceptibility to their disruption.

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A method of treating gastrointestinal stromal tumor (GIST) in a subject, comprising:
administering to the subject a therapeutically effective amount of a Menin inhibitor.

2. The method of claim 1, wherein the Menin inhibitor is JNJ-75276617, KO-539, SNDX-5613, DS-1594, or DSP-5336, MI-3454, M-808, BMF-219, A300-105A, VTP-50469, a short interfering RNA (siRNA), or a combination of two or more thereof.

3. The method of claim 2, wherein the Menin inhibitor is SNDX-5613, VTP-50469, or M-808.

4. (canceled)

5. (canceled)

6. The method of claim 1, wherein the Menin inhibitor is administered orally, intramuscularly, subcutaneously, or intravenously.

7. The method of claim 1, further comprising the step of administering to the subject a therapeutically effective amount of a tyrosine kinase inhibitor (TKI).

8. The method of claim 7, wherein the TKI is imatinib, sunitinib, regorafenib, ripretinib, nilotinib, pazopanib, cabozantinib, avapritinib, or a combination of two or more thereof.

9. The method of claim 8, wherein the TKI is imatinib.

10. The method of claim 7, wherein the TKI is administered subsequent to administration of the Menin inhibitor; or wherein the TKI is administered substantially simultaneously with administration of the Menin inhibitor; or wherein the TKI is administered prior to administration of the Menin inhibitor.

11. (canceled)

12. (canceled)

13. The method of claim 1, further comprising the step of administering to the subject a therapeutically effective amount of a MOZ inhibitor.

14. The method of claim 13, wherein the MOZ inhibitor is WM-1119, WM-8014, PF-9363, a siRNA, or a combination of two or more thereof.

15. The method of claim 14, wherein the MOZ inhibitor is WM-1119.

16. The method of claim 13, wherein the MOZ inhibitor is administered orally, intramuscularly, subcutaneously, or intravenously.

17. The method of claim 13, wherein the MOZ inhibitor is administered subsequent to administration of the Menin inhibitor; or wherein the MOZ inhibitor is administered simultaneously with administration of the Menin inhibitor; or wherein the MOZ inhibitor is administered prior to administration of the Menin inhibitor.

18. (canceled)

19. (canceled)

20. The method of claim 17, further comprising the step of administering to the subject a therapeutically effective amount of a TKI; wherein the MOZ inhibitor is administered subsequent to administration of the TKI; or wherein the MOZ inhibitor is administered substantially simultaneously with administration of the TKI; or wherein the MOZ inhibitor is administered prior to administration of the TKI.

21. (canceled)

22. (canceled)

23. The method of claim 1, wherein the subject is diagnosed with an activating mutation in or around the receptor tyrosine kinase (KIT) gene.

24. The method of claim 1, wherein the GIST is metastatic.

25. A method of reducing KIT activity in vitro or in vivo, comprising: contacting a cell having an activating mutation in or around the KIT gene with a Menin inhibitor.

26. The method of claim 25, wherein the Menin inhibitor is MI-3454, M-808, JNJ-75276617, KO-539, SNDX-5613, DS-1594, or DSP-5336, BMF-219, A300-105A, VTP-50469, or a combination of two or more thereof.

27. (canceled)

28. (canceled)

29. The method of claim 25, further comprising the step of contacting the cell with a TKI; or further comprising the step of contacting the cell with a therapeutically effective amount of a MOZ inhibitor.

30. The method of claim 29, wherein the TKI is imatinib, sunitinib, regorafenib, ripretinib, nilotinib, pazopanib, cabozantinib, avapritinib, or a combination of two or more thereof; or wherein the MOZ inhibitor is WM-1119.

31. (canceled)

32. (canceled)

33. (canceled)

34. A kit comprising a therapeutically effective amount of a Menin inhibitor, a pharmaceutically acceptable carrier disposed in a suitable container and printed instructions for using the Menin inhibitor in the treatment of GIST in a subject.

35. (canceled)

36. The kit of claim 34, further comprising a therapeutically effective amount of a TKI and printed instructions for using the TKI in the treatment of GIST in a subject, wherein the Menin inhibitor and the TKI are contained in the same dosage form or different dosage forms that are disposed in the same or different containers; or
further comprising a therapeutically effective amount of a MOZ inhibitor and printed instructions for using the MOZ inhibitor in the treatment of GIST in a subject, wherein the Menin inhibitor and the MOZ inhibitor are contained in the same dosage form or different dosage forms that are disposed in the same or different containers.

37. The kit of claim 36, wherein the TKI is imatinib; or wherein the Menin inhibitor is SNDX-5613; or wherein the MOZ inhibitor is WM-1119.

38. (canceled)


SEQUENCE LISTING (56 sequences)

SEQ ID NO: 1 - DNA, 25 bp, synthetic construct
  source 1..25: mol_type = other DNA
caccgaggggtcttaatgatccgcg

SEQ ID NO: 2 - DNA, 25 bp, synthetic construct
  source 1..25: mol_type = other DNA
aaaccgcggatcattaagacccctc

SEQ ID NO: 3 - DNA, 25 bp, synthetic construct
  source 1..25: mol_type = other DNA
caccggatccttctacttgcatggg

SEQ ID NO: 4 - DNA, 25 bp, synthetic construct
  source 1..25: mol_type = other DNA
aaaccccatgcaagtagaaggatcc

SEQ ID NO: 5 - DNA, 25 bp, synthetic construct
  source 1..25: mol_type = other DNA
caccgacgtcgtcgatggagcgcag

SEQ ID NO: 6 - DNA, 25 bp, synthetic construct
  source 1..25: mol_type = other DNA
aaacctgcgctccatcgacgacgtc

SEQ ID NO: 7 - DNA, 25 bp, synthetic construct
  source 1..25: mol_type = other DNA
caccgagagggcggcgatgatagac

SEQ ID NO: 8 - DNA, 25 bp, synthetic construct
  source 1..25: mol_type = other DNA
aaacgtctatcatcgccgccctctc

SEQ ID NO: 9 - DNA, 25 bp, synthetic construct
  source 1..25: mol_type = other DNA
caccggcttagtcgcccgcaagttg

SEQ ID NO: 10 - DNA, 25 bp, synthetic construct
  source 1..25: mol_type = other DNA
aaaccaacttgcgggcgactaagcc

SEQ ID NO: 11 - DNA, 25 bp, synthetic construct
  source 1..25: mol_type = other DNA
caccgaggcgaccctaatgcgctag

SEQ ID NO: 12 - DNA, 25 bp, synthetic construct
  source 1..25: mol_type = other DNA
aaacctagcgcattagggtcgcctc

SEQ ID NO: 13 - DNA, 25 bp, synthetic construct
  source 1..25: mol_type = other DNA
caccgtgatagccaatcgtaactgc

SEQ ID NO: 14 - DNA, 25 bp, synthetic construct
  source 1..25: mol_type = other DNA
aaacgcagttacgattggctatcac

SEQ ID NO: 15 - DNA, 25 bp, synthetic construct
  source 1..25: mol_type = other DNA
caccgtcactcgaaccgttagttca

SEQ ID NO: 16 - DNA, 25 bp, synthetic construct
  source 1..25: mol_type = other DNA
aaactgaactaacggttcgagtgac

SEQ ID NO: 17 - DNA, 25 bp, synthetic construct
  source 1..25: mol_type = other DNA
caccgcatggcgatgcacaacaagg

SEQ ID NO: 18 - DNA, 25 bp, synthetic construct
  source 1..25: mol_type = other DNA
aaacccttgttgtgcatcgccatgc

SEQ ID NO: 19 - DNA, 25 bp, synthetic construct
  source 1..25: mol_type = other DNA
caccggcggctcttcagtaaatcct

SEQ ID NO: 20 - DNA, 25 bp, synthetic construct
  source 1..25: mol_type = other DNA
aaacaggatttactgaagagccgcc

SEQ ID NO: 21 - DNA, 25 bp, synthetic construct
  source 1..25: mol_type = other DNA
caccgacgtagaccggcagcggcca

SEQ ID NO: 22 - DNA, 25 bp, synthetic construct
  source 1..25: mol_type = other DNA
aaactggccgctgccggtctacgtc

SEQ ID NO: 23 - DNA, 25 bp, synthetic construct
  source 1..25: mol_type = other DNA
caccgcgaatcgccaacacgaggta

SEQ ID NO: 24 - DNA, 25 bp, synthetic construct
  source 1..25: mol_type = other DNA
aaactacctcgtgttggcgattcgc

SEQ ID NO: 25 - DNA, 20 bp, synthetic construct
  source 1..20: mol_type = other DNA
tgcaccaccaactgcttagc

SEQ ID NO: 26 - DNA, 21 bp, synthetic construct
  source 1..21: mol_type = other DNA
ggcatggactgtggtcatgag

SEQ ID NO: 27 - DNA, 20 bp, synthetic construct
  source 1..20: mol_type = other DNA
agggtttgcacacgtccatc

SEQ ID NO: 28 - DNA, 20 bp, synthetic construct
  source 1..20: mol_type = other DNA
gtcactgctcgcacacatga

SEQ ID NO: 29 - DNA, 20 bp, synthetic construct
  source 1..20: mol_type = other DNA
ctctacgacgagagcagcag

SEQ ID NO: 30 - DNA, 20 bp, synthetic construct
  source 1..20: mol_type = other DNA
agacaccacagttcttgccc

SEQ ID NO: 31 - DNA, 19 bp, synthetic construct
  source 1..19: mol_type = other DNA
tcgcacccacactcattcg

SEQ ID NO: 32 - DNA, 21 bp, synthetic construct
  source 1..21: mol_type = other DNA
gggctacctcttgaaagtcca

SEQ ID NO: 33 - DNA, 41 bp, synthetic construct
  source 1..41: mol_type = other DNA
aatggactatcatatgcttaccgtaacttgaaagtatttcg

SEQ ID NO: 34 - DNA, 48 bp, synthetic construct
  source 1..48: mol_type = other DNA
tctactattctttcccctgcactgtgactgtgggcgatgtgcgctctg

SEQ ID NO: 35 - DNA, 91 bp, synthetic construct
  source 1..91: mol_type = other DNA
aatgatacggcgaccaccgagatctacactctttccctacacgacgctcttccgatcttcaaggtcatcttgtggaaaggacgaaacaccg

SEQ ID NO: 36 - DNA, 92 bp, synthetic construct
  source 1..92: mol_type = other DNA
aatgatacggcgaccaccgagatctacactctttccctacacgacgctcttccgatctatgcataacttcttgtggaaaggacgaaacaccg

SEQ ID NO: 37 - DNA, 93 bp, synthetic construct
  source 1..93: mol_type = other DNA
aatgatacggcgaccaccgagatctacactctttccctacacgacgctcttccgatctgatctctgatttcttgtggaaaggacgaaacaccg

SEQ ID NO: 38 - DNA, 94 bp, synthetic construct
  source 1..94: mol_type = other DNA
aatgatacggcgaccaccgagatctacactctttccctacacgacgctcttccgatctcgattacgtacgtcttgtggaaaggacgaaacaccg

SEQ ID NO: 39 - DNA, 95 bp, synthetic construct
  source 1..95: mol_type = other DNA
aatgatacggcgaccaccgagatctacactctttccctacacgacgctcttccgatcttcgattacgcgtttcttgtggaaaggacgaaacaccg

SEQ ID NO: 40 - DNA, 96 bp, synthetic construct
  source 1..96: mol_type = other DNA
aatgatacggcgaccaccgagatctacactctttccctacacgacgctcttccgatctatcgatctcagtgatcttgtggaaaggacgaaacaccg

SEQ ID NO: 41 - DNA, 97 bp, synthetic construct
  source 1..97: mol_type = other DNA
aatgatacggcgaccaccgagatctacactctttccctacacgacgctcttccgatctgatcgattctgatattcttgtggaaaggacgaaacaccg

SEQ ID NO: 42 - DNA, 98 bp, synthetic construct
  source 1..98: mol_type = other DNA
aatgatacggcgaccaccgagatctacactctttccctacacgacgctcttccgatctcgatcgatcatatgcttcttgtggaaaggacgaaacaccg

SEQ ID NO: 43 - DNA, 99 bp, synthetic construct
  source 1..99: mol_type = other DNA
aatgatacggcgaccaccgagatctacactctttccctacacgacgctcttccgatctacgatcgatcgtaattatcttgtggaaaggacgaaacaccg

SEQ ID NO: 44 - DNA, 91 bp, synthetic construct
  source 1..91: mol_type = other DNA
aatgatacggcgaccaccgagatctacactctttccctacacgacgctcttccgatcttacgtactctcttgtggaaaggacgaaacaccg

SEQ ID NO: 45 - DNA, 100 bp, synthetic construct
  source 1..100: mol_type = other DNA
caagcagaagacggcatacgagatcaaggtcagtgactggagttcagacgtgtgctcttccgatctacgatcgattctactattctttcccctgcactgt

SEQ ID NO: 46 - DNA, 99 bp, synthetic construct
  source 1..99: mol_type = other DNA
caagcagaagacggcatacgagatgcataactgtgactggagttcagacgtgtgctcttccgatctcgatcgattctactattctttcccctgcactgt

SEQ ID NO: 47 - DNA, 98 bp, synthetic construct
  source 1..98: mol_type = other DNA
caagcagaagacggcatacgagatctctgattgtgactggagttcagacgtgtgctcttccgatctgatcgattctactattctttcccctgcactgt

SEQ ID NO: 48 - DNA, 97 bp, synthetic construct
  source 1..97: mol_type = other DNA
caagcagaagacggcatacgagattacgtacggtgactggagttcagacgtgtgctcttccgatctatcgattctactattctttcccctgcactgt

SEQ ID NO: 49 - DNA, 96 bp, synthetic construct
  source 1..96: mol_type = other DNA
caagcagaagacggcatacgagattacgcgttgtgactggagttcagacgtgtgctcttccgatcttcgattctactattctttcccctgcactgt

SEQ ID NO: 50 - DNA, 95 bp, synthetic construct
  source 1..95: mol_type = other DNA
caagcagaagacggcatacgagatctcagtgagtgactggagttcagacgtgtgctcttccgatctcgattctactattctttcccctgcactgt

SEQ ID NO: 51 - DNA, 94 bp, synthetic construct
  source 1..94: mol_type = other DNA
caagcagaagacggcatacgagattctgatatgtgactggagttcagacgtgtgctcttccgatctgattctactattctttcccctgcactgt

SEQ ID NO: 52 - DNA, 93 bp, synthetic construct
  source 1..93: mol_type = other DNA
caagcagaagacggcatacgagatcatatgctgtgactggagttcagacgtgtgctcttccgatctattctactattctttcccctgcactgt

SEQ ID NO: 53 - DNA, 92 bp, synthetic construct
  source 1..92: mol_type = other DNA
caagcagaagacggcatacgagatcgtaattagtgactggagttcagacgtgtgctcttccgatctttctactattctttcccctgcactgt

SEQ ID NO: 54 - DNA, 100 bp, synthetic construct
  source 1..100: mol_type = other DNA
caagcagaagacggcatacgagatacgtactcgtgactggagttcagacgtgtgctcttccgatctacgatcgattctactattctttcccctgcactgt

SEQ ID NO: 55 - DNA, 24 bp, synthetic construct
  source 1..24: mol_type = other DNA
aatgatacggcgaccaccgagatc

SEQ ID NO: 56 - DNA, 24 bp, synthetic construct
  source 1..24: mol_type = other DNA
caagcagaagacggcatacgagat